(19) United States
(12) Patent Application Publication
(10) Pub. No.: US 2026/0199350 A1
(43) Pub. Date: Jul. 16, 2026

(54) FLAVONES, QUINOLINONES, QUINAZOLINONES, AND AURONES, AND USES IN CK2-MEDIATED DISEASES

(71) Applicant: Salk Institute for Biological Studies, La Jolla, CA (US)

(72) Inventors: Ioana Ilinca Nitulescu, San Diego, CA (US); Fred H. Gage, La Jolla, CA (US); James K. Tucker, San Diego, CA (US)

(73) Assignee: Salk Institute for Biological Studies, La Jolla, CA (US)

(21) Appl. No.: 19/131,341

(22) PCT Filed: Nov. 21, 2023
(86) PCT No.: PCT/US2023/080655

§ 371 Date: May 20, 2025

Related U.S. Application Data
(60) Provisional application No. 63/384,656, filed on Nov. 22, 2022.

(51) Int. Cl. A61K 31/519; A61K 31/343; A61K 31/352; A61K 31/41; A61K 31/436; A61K 31/4433; A61K 31/4709; A61K 31/506; A61K 31/517; A61P 25/28; C07D 215/233; C07D 239/91; C07D 307/83; C07D 311/28; C07D 401/04; C07D 405/04; C07D 405/06; C07D 405/10; C07D 407/10; C07D 471/04; C07D 491/04; C12N 9/99

(52) U.S. Cl. CPC A61K 31/519; A61K 31/343; A61K 31/352; A61K 31/41; A61K 31/436; A61K 31/4433; A61K 31/4709; A61K 31/506; A61K 31/517; A61P 25/28; C07D 215/233; C07D 239/91; C07D 307/83; C07D 311/28; C07D 401/04; C07D 405/04; C07D 405/06; C07D 405/10; C07D 407/10; C07D 471/04; C07D 491/04; C12N 9/99

(57) ABSTRACT

Flavone, quinolinone, quinazolinone, and aurone derivatives are disclosed. The compounds have a structure according to formula I or formula II, or a pharmaceutically acceptable salt, hydrate, stereoisomer, or tautomer thereof. The compounds may inhibit CK2 enzyme activity. Some compounds selectively reduce or inhibit the CK2A2 enzyme subunit over the CK2A1 enzyme subunit.

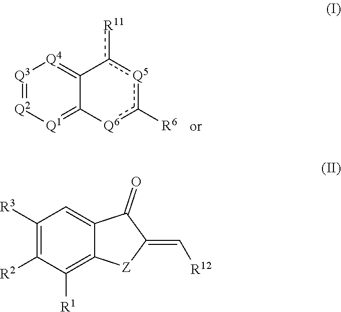

### CROSS-REFERENCE TO RELATED APPLICATION

This application claims the benefit of the earlier filing date of U.S. Provisional Application No. 63/384,656, filed Nov. 22, 2022, which is incorporated in its entirety herein by reference.

### FIELD

This disclosure concerns flavones, quinolinones, quinazolinones, and aurones, and their uses for CK2-mediated diseases, particularly CK2A2-mediated diseases.

### INCORPORATION OF ELECTRONIC SEQUENCE LISTING

The Sequence Listing is submitted as an XML file named “Sequence.xml,” created on Nov. 16, 2023, 4,613 bytes, which is incorporated by reference herein.

### BACKGROUND

Neuroinflammation has emerged as a significant etiological factor for numerous neurological and psychiatric diseases, including Alzheimer's disease (AD), Huntington's disease (HD), and Parkinson's disease (PD). Microglia and astrocytes, the brain's resident immune cells, become chronically activated and perpetuate a destructive loop of pro-inflammatory cytokine release. Inflammation and mitochondrial dysfunction are two known processes that contribute to AD by influencing the pathogenesis and severity. Over time, chronic inflammation propagated by microglia and astrocytes leads to neurotoxicity and defects in normal brain function. Notably, inflammation might precede neuronal loss, the hallmark of many neurodegenerative diseases. In turn, mitochondrial dysfunction in neurons, which rely on oxidative phosphorylation for energy, leads to neuronal death. Chronic inflammation in AD is evidenced by the presence of reactive glia in postmortem studies and inflammatory biomarkers in serum and cerebrospinal fluid (CSF).

Casein kinase II (CK2) is a kinase with a brain-enriched variant that shows increased activity in inflammatory and neurodegenerative diseases. Remarkably, CK2 regulates mitochondrial homeostasis as well as innate immune pathways. In neurons, defects in mitophagy and mitochondrial fragmentation have been linked to degeneration in AD patients as well as AD mouse models. Protein kinase CK2 regulates both of these AD-associated processes. First, CK2 regulates innate immunity pathways, and CK2 inhibitors have shown efficacy in immune-driven cancers. CK2 protein levels increase in peripheral immune cells upon induction by pro-inflammatory stimuli such as LPS and TNFα, and CK2 phosphorylates important mediators of inflammation, including NF-κB, IκBα, and AKT. Second, CK2 regulates two aspects of mitochondrial homeostasis, mitophagy and mitochondrial fission. CK2 blocks mitophagy through phosphorylation and inactivation of FUNDC1, which leads to accumulation of damaged mitochondria and mitochondrial apoptosis. CK2 also indirectly upregulates phosphorylation of MFF, leading to mitochondrial fission. In models of vascular injury, CK2 knockdown and CK2 inhibitor treatment restores mitophagy and blocks mitochondrial fission.

Finally, CK2 is highly expressed in the brain and one of its catalytic subunits, CK2A2, is enriched in the brain relative to other tissues. There is evidence of dysregulation of CK2 at the gene and protein level in neurodegenerative diseases. For example, higher CK2 levels were observed in astrocytes from AD patients. In PD, CK2 regulatory subunits were reported to co-localize with Lewy bodies. Notably, CK2 overexpression causes cognitive decline in wild-type mice, and higher CK2 levels and/or activity was observed in transgenic mice for AD (APP/PS1 and 3×Tg models), PD (alpha-synuclein A53T model), and Huntington's disease (zQ175 model).

### SUMMARY

Flavone, quinolinone, quinazolinone, and aurone derivatives are disclosed. The compounds may be useful for treating CK2-mediated diseases. In some aspects, the compounds are useful for treating diseases mediated by upregulation of the CK2A2 enzyme subunit and/or exhibit selectivity for the CK2A2 enzyme submit over the CK2A1 enzyme subunit.

In some aspects, the disclosed compounds have a structure formula I or formula II, or a pharmaceutically acceptable salt, hydrate, stereoisomer, or tautomer thereof:

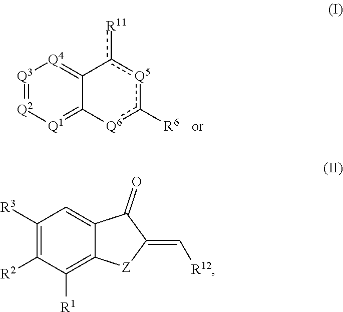

where each bond represented by “ [symbol] ” is a single or double bond as needed to satisfy valence requirements. With respect to formulas I and II, Q¹ is C—R¹ or N; Q² is C—R² or N; Q³ is C—R³ or N; Q⁴ is C—R⁴ or N; Q⁵ is C—R⁵, N, or N—R^{b}; Q⁶ is 0, N, or N—R^{b}; Z is O, S, or N—R^{b}; R¹ is X or H; R² is H or —OR^{a}; R³ is X or H; R⁴ is H; R is H; R is

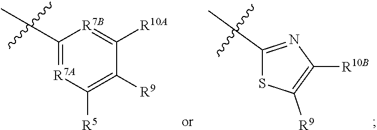

R^{7A} and R^{7B} independently are N or CH; R⁸ is H or X; R⁹ is hydroxy; R^{10A} and R^{10B} independently are X, —OR^{a}, C₁-C₅ aliphatic, substituted C₁-C₅ aliphatic, or cycloaliphatic; R¹¹ is O, —OR^{a}, or N(R^{a})₂; R¹² is

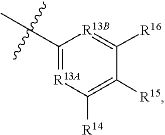

furan-2-yl, or furan-3-yl; R^{13A} and R^{13B} independently are N or CH; R¹⁴ is H, X, or —OR^{a}; R¹⁵ is —OR^{a}, H, cyano, tetrazolyl, or —CO₂R^{a}; R¹⁶ is H, X, —OR^{a}, substituted aliphatic, cycloaliphatic, or alkynyl; each R^{a} independently is H or C₁-C₅ aliphatic; each R^{b} independently is H, C₁-C₅ aliphatic, or cycloaliphatic; an X is halo. When the compound has a structure according to formula I, at least one of Q¹-Q⁴ is N, or Q⁵ is N or N—R^{b}, or Q⁶ is N or N—R^{b}, or R¹ is other than O.

In some aspects, the compound has a structure according to any of formulas I-A to I-Q or II-A:

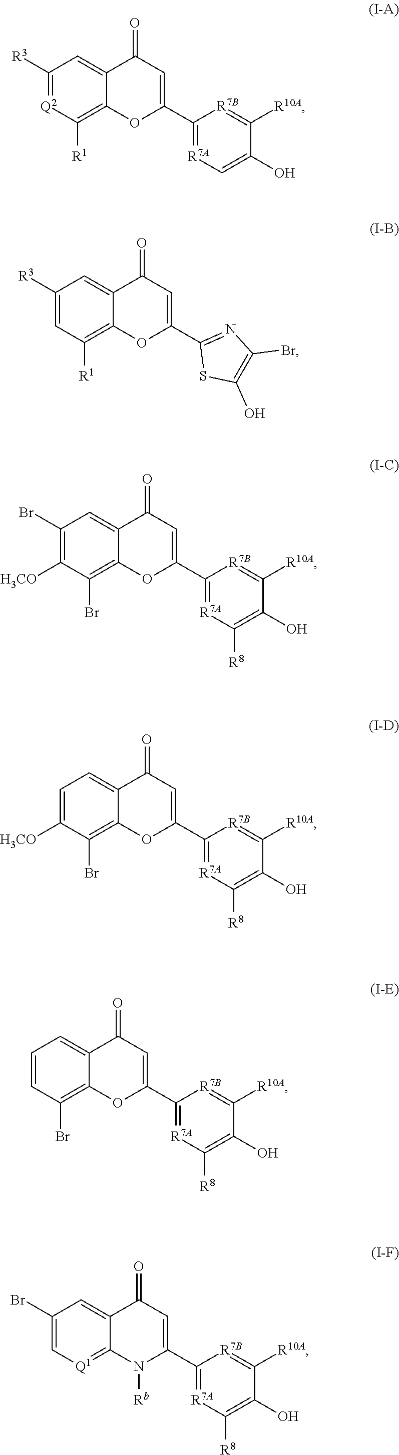

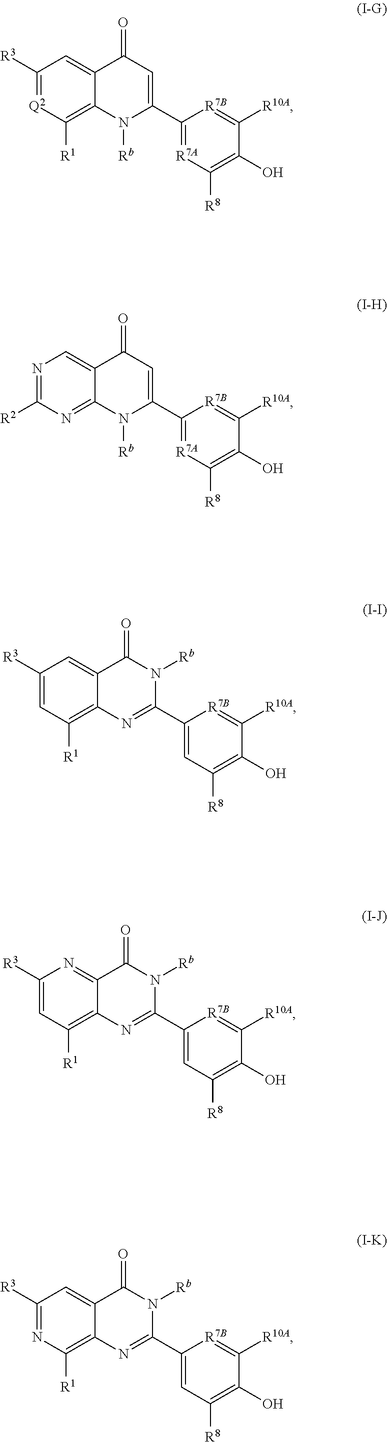

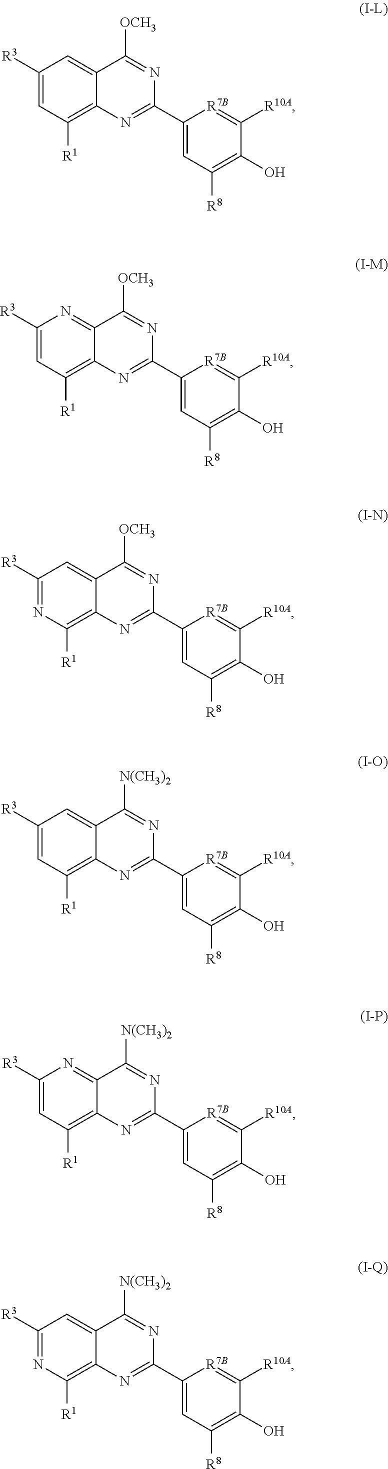

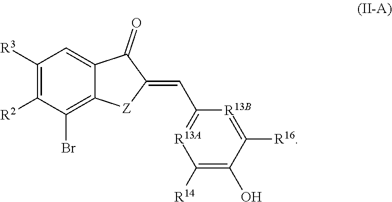

In certain implementations, the compound has an IC₅₀ for CK2A2 that is less than 50% of the IC₅₀ for CK2A1. This disclosure also encompasses pharmaceutical compositions comprising a compound as disclosed herein, or a pharmaceutically acceptable salt, hydrate, stereoisomer, or tautomer thereof, and a pharmaceutically acceptable excipient.

A method of reducing CK2 enzyme activity includes contacting a cell that expresses CK2 enzyme with an effective amount of one or more compounds disclosed herein, thereby reducing activity of the CK2 enzyme. In some aspects, CK2A2 expression is upregulated in the cell. In certain implementations, the cell is an astrocyte, a microglia, a neuron, a white blood cell, an adipocyte, a myocyte, or an epithelial cell.

In any of the foregoing or following aspects, contacting the cell with the one or more compounds may comprise administering a therapeutically effective amount of the one or more compounds, or an amount of a pharmaceutical composition comprising the therapeutically effective amount of the one or more compounds, to a subject. The subject may have a disease or condition characterized at least in part by dysregulated CK2 enzyme activity. In some aspects, the disease or condition is characterized at least in part by inflammation. Exemplary diseases and conditions include, but are not limited to, a neurodegenerative disease, cancer, cardiac hypertrophy, cystic fibrosis, bipolar disorder, depression, a viral infection, obesity, diabetes mellitus, atherosclerosis, epilepsy, or any combination thereof.

The foregoing and other objects and features of the disclosure will become more apparent from the following detailed description, which proceeds with reference to the accompanying figures.

### BRIEF DESCRIPTION OF THE DRAWINGS

FIGS. 1A-lE characterize flavones for anti-inflammatory activity and cytotoxicity in astrocytes (5 hour treatment). FIG. 1A—SAR studies show divergent activity of flavones and flavanones in a flow-cytometry-based assay of cytokine production. Each dot represents one biological sample from 2-5 independent experiments (n=2-12, mean±SD, one-way ANOVA with Bonferroni post-hoc test). Bar charts for NAR and chrysin in the IL6 graph represent one experiment. FIG. 1B—NAR does not block IL-6, IL-8, MCP-1, and TNFα cytokine production in HCA. Representative flow cytometry traces from 2 independent experiments in duplicate. FIG. 1C —Dose-response curve of IL-6 expression in TMF or dTMF treated astrocytes activated with IL1-β (n=3-6, mean±SD), representative of 3 independent experiments. FIG. 1D—Normalized expression (IL6/GAPDH) relative to DMSO from a ddPCR experiment showing no effect of dCHR on IL6 expression in IL1-β-activated astrocytes (n=1-2 biological samples, Poisson error of >15,000 droplets). FIG. 1E—Flavones do not reduce cell viability of primary human astrocytes at 20 μM. Each dot represents the percentage of non-Zombie Violet stained cells in one biological sample from 2-5 independent experiments (n=2-10, mean±SD).

FIGS. 2A-2F show anti-inflammatory activity and unbiased target identification of flavones in glia. FIG. 2A—Apigenin (API) blocks IL6, IL8, MCP-1, and TNFα cytokine production in human primary astrocytes (HCA). Representative flow cytometry traces from 12 independent experiments. FIG. 2B—Chrysoeriol (CHR) suppresses LPS-induced pro-inflammatory cytokine release and boosts anti-inflammatory cytokine release in human iPSC-derived microglia (n=2, mean fold-change). Representative of 2 independent experiments. FIG. 2C—CHR 20 μM blocks microglial phagocytosis of E coli-FITC conjugated beads (5 h treatment, n=2-3, mean±SD, one-way ANOVA with Dunnett's post-hoc test). Representative of 2 independent experiments. FIG. 2D —CHR exhibits dose-dependent anti-inflammatory activity in human astrocytes and microglia by blocking IL1-β or LPS-induced IL-6 secretion, respectively (n=6, mean±s.e.m.). Normalized to DMSO vehicle±stimulation; 2 independent experiments pooled for analysis. FIG. 2E—Thermal shift proteome profiling (TPP) in human iPSC-derived astrocytes. Red denotes proteins exhibiting significant and reproducible thermal shifts after CHR treatment in IL1-β-activated astrocytes. FIG. 2F—Isothermal dose response thermal shift assay validates CK2 as a target of CHR. Each dot represents normalized band intensity (Western blot). Representative of two independent experiments. *P<0.05; **P<0.01; ***P<0.001; ****P<0.0001.

FIGS. 3A-3F show thermal shift proteome profiling and Western blotting demonstrating stabilization of CK2 and CK2 interactors by flavones. FIG. 3A—TSA-WB isothermal dose response showing stabilization of CK2α1 by CHR (see FIG. 2F). FIG. 3B—GeneMania network showing protein-protein interactions between hits from FIG. 2E (red nodes-stabilized and blue nodes-destabilized) and related proteins (white nodes). FIG. 3C—Immunoblot showing physical interaction of PTGR1 and CK2α1 by co-immunoprecipitation. FIG. 3D—TPP in human iPSC-derived astrocytes. Red denotes proteins exhibiting significant and reproducible thermal shifts after TMF treatment in IL1-β-activated astrocytes. FIG. 3E—GeneMania network showing protein-protein interactions between hits from FIG. 1C (red nodes-stabilized, blue nodes-destabilized, gray nodes—not significant) and related proteins (white nodes). FIG. 3F—TSA-WB (left) with TMF and dTMF, along with band intensity quantification (right).

FIGS. 4A-4F characterize flavones for in vitro and in-cell CK2 inhibition. FIG. 4A —Kinase capture shows apigenin is an ATP-competitive inhibitor of CK2α1 and CK2α2 in H1-derived astrocyte whole lysates. Graph shows band intensity normalizations to a 150 kD protein stained by Amido Black; lines represent nonlinear curve fits of dose-curve data. Representative of two independent experiments. FIG. 4B—NanoBRET assay shows dose-dependent CK2α1 and CK2α2 target engagement in HEK293T cells (each point mean±sem, n=3). Nonlinear fit was used to calculate IC₅₀. FIG. 4C—NanoBRET assay shows dose-dependent CK2α1 and CK2α2 target engagement of chrysin in HEK293T cells, but not NAR, dCHR, dTMF, or dO-TMF (each point mean±sem, n=3). Nonlinear fit was used to calculate IC₅₀. FIG. 4D—No activity of chrysin, NAR, dCHR, dTMF, or dO-TMF in THP-1-NF-κB-Lucia monocytes (each point mean±sem, n=3). Nonlinear fit was used to calculate IC₅₀. FIG. 4E—Radiometric kinase assays using recombinant CK2α1 do not show inhibitory activity of NAR, dCHR, dTMF, or dO-TMF. FIG. 4F—Structurally unrelated CK2 inhibitor CX-4945 blocks IL1-β-induced IL-6 upregulation as well as TMF (mean±SD, n=2, one-way ANOVA with Tukey's post-hoc test). Normalized to DMSO, data are representative of three independent experiments.

FIGS. 5A-5G show that inhibition of CK2 kinase activity reduces inflammation in glia. FIG. 5A—Summary of SAR data shows that API, CHR, luteolin, quercetin, kaempferol, fisetin and TMF are active, chrysin is intermediately active, and dTMF, dCHR, NAR, and dO-TMF are inactive at 20 μM both in an NF-κB reporter assay and for CK2 kinase inhibition. Asterisk denotes values sourced from Baier et al. (2017) and Lolli et al. (2012). FIG. 5B—Most active flavones dose-dependently reduce NF-κB activity in THP-1 macrophages (LUT=luteolin, QUE=quercetin, FIS=fisetin, KAE=kaempferol). FIG. 5C—Structurally unrelated CK2 inhibitor CX-4945 blocks IL1β-induced IL-6 upregulation (mean±s.e.m., n=3). Normalized to DMSO, data are representative of two independent experiments. FIG. 5D—KD of CK2α1 and CK2α2 significantly blocks IL1-β-induced IL-8 upregulation. Box plot with mean values and 25-75% CI of 4-5 independent experiments, total n=4-8 biological replicates, unpaired t-test). FIG. 5E —Overexpression of CK2 kinase-dead mutants significantly blocks IL1-3-induced IL-8 upregulation (means of three independent experiments shown with Poisson error); each value normalized within experiments to WT. FIG. 5F—Quantification of NF-κB reporter luciferase activity in CK2A1 or CK2A2 heterozygous (het) or homozygous (KO) knockout THP-1 NF-κB Lucia cell clones stimulated with various doses of LPS (mean±sem, n=3). FIG. 5G—Quantification of NF-κB reporter luciferase activity in CK2A2 homozygous (KO) knockout THP-1 NF-κB Lucia cell clones stimulated with 20 ng/mL LPS and CHR dilution series (mean±sem, n=3). *P<0.05; **P<0.01; ***P<0.001; ****P<0.0001.

FIGS. 6A-6G show results of CK2 genetic perturbation assays and NF-κB immunoprecipitation. FIG. 6A—Knockdown efficiency of experiments shown in FIG. 5B (mean expression shown with each dot representing an experiment). FIG. 6B—Expression of CK2α1-HA and CK2α2-HA confirmed by WB. FIG. 6C—CK2 kinase-dead mutants block IL1-β-induced IL-8 upregulation in HCA (3 independent experiments shown, each value normalized within experiments to own WT; mean±sem, n=3). FIG. 6D—Western blotting showing expression of CK2A1 and GAPDH in individual THP-1 NF-κB-Lucia clones, quantified as a relative ratio. Heterozygotes and homozygote confirmed by Sanger sequencing. FIG. 6E—Western blotting showing CK2A2 expression in individual WT or KO THP-1 NF-κB-Lucia clones after immunoprecipitation of CK2A2. FIG. 6F—Quantification of NF-κB reporter luciferase activity in CK2A1 heterozygous (het) or homozygous (KO) knockout THP-1 NF-κB Lucia cell clones stimulated with 20 ng/mL LPS and CHR dilution series (mean±sem, n=3). FIG. 6G —Immunoblot of NF-κB IP showing NF-κB S529 phosphorylation is reduced with CHR (2 h treatment).

FIGS. 7A-7H show that CK2 inhibition attenuates inflammatory biochemical and transcriptional programs via NF-κB. FIG. 7A—CK2 levels increase with inflammation (time course by Western). FIG. 7B—Immunofluorescence images showing reduction of phospho-CK2α1 Y255/CK2α1 after 5 h of CK2 inhibitor treatment (mean±SD, n=4, one-way ANOVA, Fisher's test). FIG. 7C—Immunoblots showing that pIkB/IkBa levels are reduced with CK2 inhibitors (6 h treatment, mean±SD, n=2). FIG. 7D—Immunofluorescence images showing reduction of phospho-NF-κB S529/NF-κB after 5 h of CK2 inhibitor treatment (mean±SD, n=2-4, one-way ANOVA, Fisher's test). FIG. 7E—Quantification of NF-κB reporter luciferase activity in a pool of THP-1 NF-κB Lucia NF-κB-S529A NeoR knockin cells or parental wild-types stimulated with various doses of LPS (mean of n=3 biological replicates shown). FIG. 7F—X2K interaction network showing top enriched kinase modules, intermediate proteins (not labeled), and their downstream TF targets in IL1-β-stimulated astrocytes (unrelated to CK2 in gray, red edges denote CK2 interactions). FIG. 7G—Immune signatures are significantly enriched in genes downregulated in inflamed astrocytes treated with APL FIG. 7H—CK2 inhibition reduces expression of neurotoxic A1 genes and increases expression of neuroprotective A2 genes.

FIGS. 8A-8E show RNA-seq in IL1-β and API-treated astrocytes. FIG. 8A—Bar graph depicting p-values associated with enriched kinases in IL1-β-treated astrocytes compared to controls (KEA). FIG. 8B—Bar graph depicting p-values associated with enriched TFs and other chromatin factors in IL1-β-treated astrocytes compared to controls (ChEA). FIG. 8C—Venn diagram showing overlap of significantly DE genes up in IL1-β and down in API+IL1-β treated cells. FIG. 8D—Venn diagram showing overlap of significantly DE genes down in IL1-β and up in API+IL1-β treated cells. FIG. 8E—Rainfall plot showing rank of CMap signatures by z-score in terms of similarity to API+IL1-β-treated HCAs.

FIGS. 9A-9I show that CK2 is dysregulated in Alzheimer's disease and correlates with neuroinflammation and cognitive decline. FIG. 9A—Left: Expression of CK2 genes in postmortem brains (parietal cortex) from patients with dementia (Dem, n=21) or controls (Ct, n=27) from the Aging, Dementia, and TBI RNA-seq study. Box plot depicting mean z-score (two-tailed t-test). Right: GFAP levels (x-axis) plotted against CK2A2 expression (y-axis) in the same patients. FIG. 9B—CSNK2A2 protein levels in AD are higher than controls, data from the UPP Proteomics Study syn17009177. Box plot depicting mean log 2(abundance) (n=44-48, one-way ANOVA with Sidak's post-hoc test); see FIG. 10A for plot of CSNK2A1 and CSNK2B (not significant). FIG. 9C Graphs depicting the top 2 annotation clusters and their associated terms and −log(p-values) from DAVID gene ontology analysis of significantly AD-upregulated genes. FIG. 9D—Bar plots showing counts per million (CPM) CSNK2A2 mRNA expression by RNA-seq (mean and 5-95% CI of n=3 AD and n=3 CT samples in triplicate). FIG. 9E—Representative immunoblots and quantification showing cellular CK2α1 and CK2α2 protein levels normalized to GAPDH in AD vs. control iPSC-derived astrocytes (mean and 5-95% CI of n=6 AD and n=4 CT samples in duplicate, p=0.027). FIG. 9F—Representative immunoblots showing pNF-κB, NF-κB, and actin expression in three AD astrocyte cell lines (5 h treatment) and quantification of pNF-κB/NF-κB baseline levels (mean and 5-95% CI of n=7 AD and n=4 CT samples; t-test). Representative of three independent experiments. FIG. 9G—STRING network of top 20 significantly enriched kinases by KEA of significantly AD-upregulated genes (p-values shown for CK2 isoforms). FIG. 9H—Box plots showing IL-6 and TNFAIP2 fold-change (mean and 5-95% CI of two pooled sets of n=5-6 AD and n=3-4 CT cell lines in duplicate; two-tailed t-test). Pool of two independent experiments. FIG. 91—Box plots showing IL-6 and TNFAIP2 mRNA expression relative to GAPDH (mean and 5-95% CI of n=5 AD and n=3 CT samples in duplicate; repeated measures ANOVA with Sidak's multiple comparisons test). Representative of three independent experiments. *P<0.05; **P<0.01; ***P<0.001; ****P<0.0001.

FIGS. 10A-10C show that CK2α2 levels are higher in AD postmortem samples and AD patient-derived astrocytes. FIG. 10A—CSNK2A2 protein levels in AD are higher than controls, from the UPP Proteomics Study. Box plot depicting mean log 2(abundance) (n=44-48, one-way ANOVA with Sidak's post-hoc test), CSNK2A1 and CSNK2B not significant. FIG. 10B—Representative images of immunostainings of astrocytes expressing astrocyte markers: Vimnentin, S100b and Glast. Quantification of the fraction of astrocytes expressing astrocyte markers reveals a homogeneous population of iPSC-derived astrocytes from both AD and controls. n.s.: not significant. Data represent mean±SEM for n=5 randomized images counted blindly for each line (total of 6 lines: 3 AD and 3 controls). FIG. 10C—The rest of the blots included in the quantification from FIG. 9F. Cell lines 40, 107, 52, and 26 are controls, while the rest of the cell lines are AD.

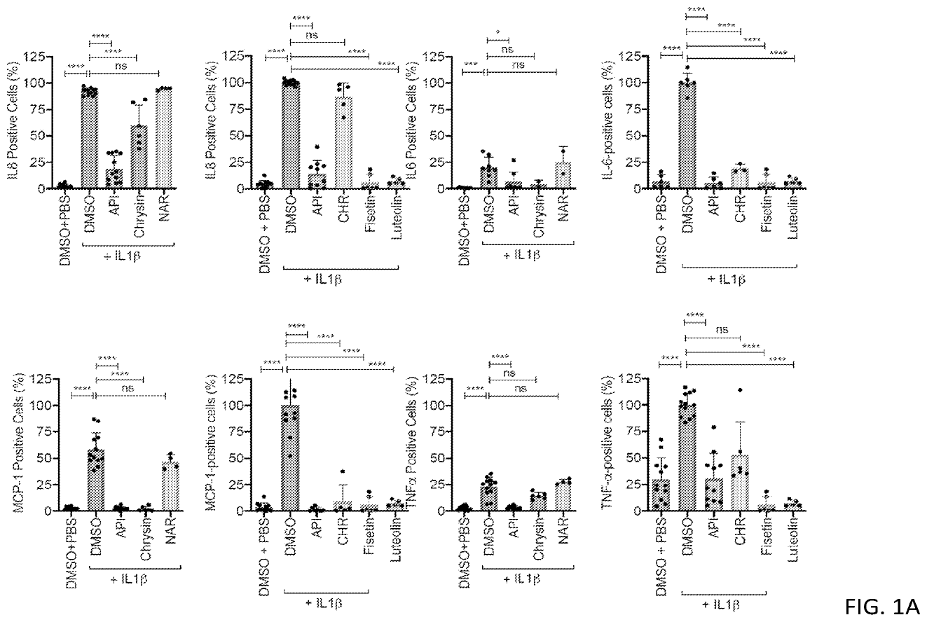

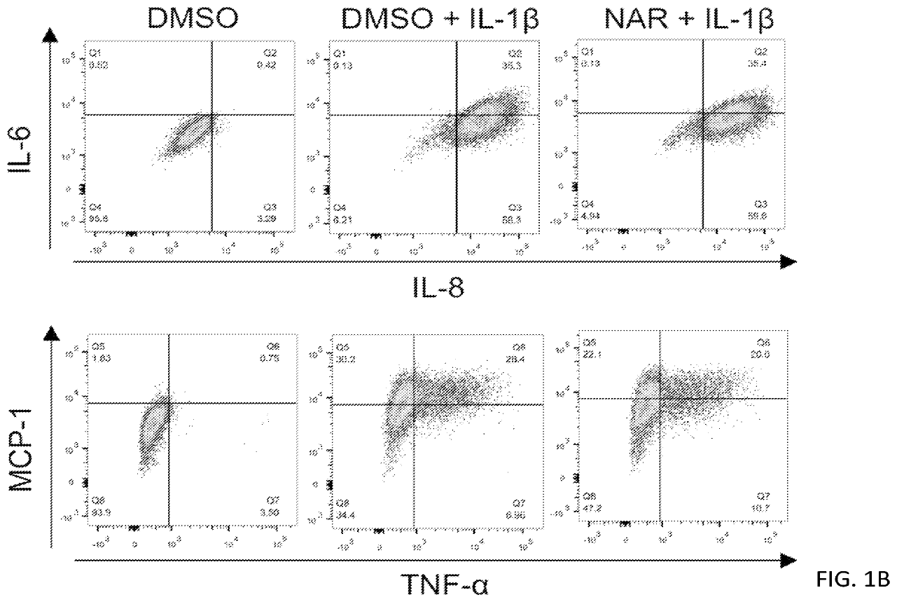

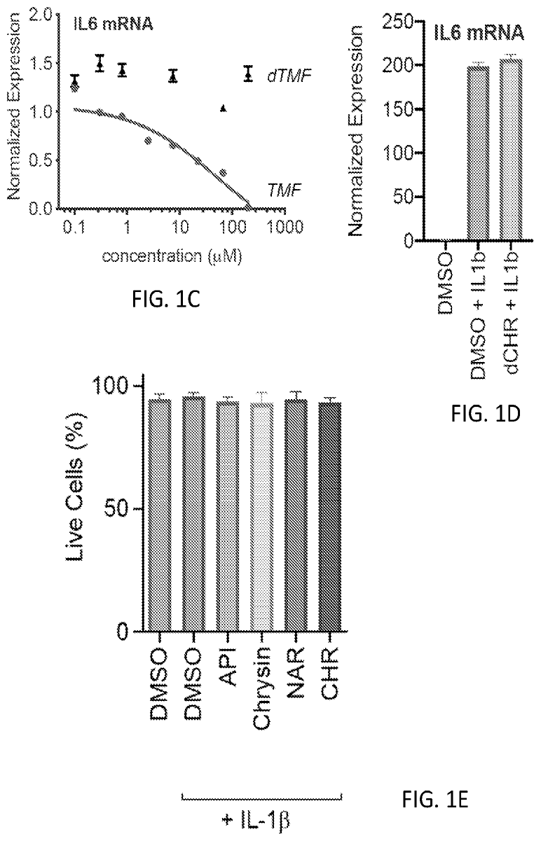

FIG. 1I demonstrates that mechanistic studies with CRISPR-Cas9 show dependency on CK2A1 and CK2A2 for inflammatory activation.

FIG. 12 demonstrates significant rescue of CHR anti-inflammatory effect in CK2A2-KO cells.

FIG. 13 demonstrates CK2A2 knockout leads to reduction in AKT S129 phosphorylation, while CK2A1 knockout does not. This suggests that it is a CK2A2-specific biomarker.

FIG. 14 shows that CK2 inhibitor CHR reduces tau phosphorylation in induced neurons (iNs).

FIG. 15 shows that CK2 inhibitors JKT.177 and JKT.606 elevates marker of mitochondrial mass ACO2 and marker of autophagy LC3-II.

### SEQUENCES

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand. In the accompanying sequence listing:

SEQ ID NO: 1 is a DNA sequence encoding a
synthetic gRNA
GTGAGGATAGCCAAGGTTCT.
SEQ ID NO: 2 is a DNA sequence encoding a
synthetic gRNA
ACGCCGAGGTGAACAGTCTG.
SEQ ID NO: 3 is a DNA sequence encoding a
synthetic gRNA
ATCTCCTGAAAGGAGGCCAT.
SEQ ID NO: 4 is a DNA sequence encoding a
synthetic gRNA
AGGGCAGGCGTCACCCCCTT.

### DETAILED DESCRIPTION

This disclosure concerns flavone, quinolinone, quinazolinone, and aurone derivatives and their uses in CK2-mediated diseases, particularly diseases mediated by upregulation of the CK2A2 enzyme subunit. In some aspects, the compounds exhibit selectivity for the CK2A2 enzyme subunit over the CK2A1 enzyme subunit.

### I. TERMS

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). The presently disclosed compounds also include all isotopes of atoms present in the compounds, which can include, but are not limited to, deuterium, tritium, ¹⁸F, ¹⁴C, etc. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of the disclosure, the following explanations of specific terms are provided:
Administration: To provide or give a subject an agent, such as one or more compounds according to formulas I-IV provided herein, by any effective route. Exemplary routes of administration include, but are not limited to, oral, injection (such as subcutaneous, intramuscular, intradermal, intraperitoneal, intravenous, intraosseous, intracerebroventricular, intrathecal, and intratumoral), sublingual, rectal, transdermal, intranasal, vaginal and inhalation routes.
Aliphatic: A substantially hydrocarbon-based compound, or a radical thereof (e.g., C₆H₁₃, for a hexane radical), including alkanes, alkenes, alkynes, including cyclic versions thereof, and further including straight- and branched-chain arrangements, and all stereo and position isomers as well. Cyclic versions are commonly referred to as cycloaliphatic. Unless expressly stated otherwise, an aliphatic group contains from one to twenty-five carbon atoms; for example, from one to fifteen, from one to ten, from one to six, or from one to four carbon atoms. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group). A substituted aliphatic group includes at least one sp³-hybridized carbon or two sp²-hybridized carbons bonded with a double bond or at least two sp-hybridized carbons bonded with a triple bond. Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality.
Alkoxy: A radical (or substituent) having the structure —OR, where R is a substituted or unsubstituted alkyl. Methoxy (—OCH₃) is an exemplary alkoxy group.
Alkyl: A hydrocarbon group having a saturated carbon chain. The chain may be cyclic, branched or unbranched. Examples, without limitation, of alkyl groups include methyl, ethyl, propyl, butyl, pentyl, hexyl, heptyl, octyl, nonyl and decyl. The terms alkenyl and alkynyl refer to hydrocarbon groups having carbon chains containing one or more double or triple bonds, respectively.
Analog or Derivative: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a compound that is derived from a similar compound or a compound that can be imagined to arise from another compound, for example, if one atom is replaced with another atom or group of atoms. The latter definition is common in organic chemistry. In biochemistry, the word is used for compounds that at least theoretically can be formed from the precursor compound.
Casein kinase 2 (CK2): (e.g., OMIM: 115440 and 115441) A serine/threonine-selective protein kinase responsible for phosphorylation of substrates in various pathways within a cell, and has been implicated in cell cycle control, DNA repair, regulation of the circadian rhythm, and other cellular processes. A member of enzyme class EC 2.7.11.1. CK2 typically appears as a tetramer of two a subunits and two β subunits. The terms CK2A, CK2a, and CK2α as used herein are interchangeable. Similarly, the terms CK2B, CK2b, and CK2β are interchangeable.
COVID-19: A contagious disease caused by severe acute respiratory syndrome coronavirus 2 (SARS-CoV-2). Symptoms of COVID-19 are variable, but often include fever, cough, fatigue, breathing difficulties, and loss of smell and taste. Symptoms can begin one to fourteen days after exposure to the virus. Around one in five infected individuals do not develop any symptoms. While most people have mild symptoms, some people develop acute respiratory distress syndrome (ARDS). ARDS can be precipitated by cytokine storms, multi-organ failure, septic shock, and blood clots. Longer-term damage to organs (in particular, the lungs and heart) has been observed. There is concern about a significant number of patients who have recovered from the acute phase of the disease but continue to experience a range of effects—known as long COVID—for months afterwards. These effects include severe fatigue, memory loss and other cognitive issues, low-grade fever, muscle weakness, and breathlessness. In some examples, one or more of the compounds disclosed herein are used to treat COVID-19.
Excipient: A physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Examples of excipients include but are not limited to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.
Pharmaceutically acceptable: A substance that can be taken into a subject without significant adverse toxicological effects on the subject. The term “pharmaceutically acceptable form” means any pharmaceutically acceptable derivative or variation, such as stereoisomers, stereoisomer mixtures, enantiomers, solvates, hydrates, isomorphs, polymorphs, pseudomorphs, neutral forms, salt forms, and prodrug agents.
SARS-CoV-2: SARS-CoV-2 is a positive-sense, single stranded RNA virus of the genus betacoronavirus. The term SARS-CoV-2 includes the original SARS-Cov-2 virus or variants thereof (such as the UK variant B1.1.17, the South Africa variant B.1.351, the Brazil variant P.1, the BA.4 (delta) variant, and the BA.5 (omicron) variants). The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′—spike (S)—envelope (E)—membrane (M) and nucleocapsid (N)—3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days. In some examples, one or more of the compounds disclosed herein are used to treat SARS-CoV-2 infections.
Stereoisomers: Isomers that have the same molecular formula and sequence of bonded atoms, but which differ only in the three-dimensional orientation of the atoms in space.
Subject: An animal (human or non-human) subjected to a treatment, observation or experiment. Includes both human and veterinary subjects, including human and non-human mammals, such as rats, mice, cats, dogs, pigs, horses, cows, and non-human primates. In some examples, the treated subject has an inflammatory disease or a disease that causes undesired inflammation, such as cancer, cardiac hypertrophy, cystic fibrosis, a neurodegenerative disease, bipolar disorder, depression, a viral infection (such as SARS-CoV-2), obesity, diabetes mellitus, atherosclerosis, epilepsy, or any combination thereof.
Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.
Substituted: A fundamental compound, such as an aryl or aliphatic compound, or a radical thereof, having coupled thereto one or more substituents, each substituent typically replacing a hydrogen atom on the fundamental compound. A person of ordinary skill in the art will recognize that compounds disclosed herein may be described with reference to particular structures and substituents coupled to such structures, and that such structures and/or substituents also can be further substituted, unless expressly stated otherwise or context dictates otherwise. Solely by way of example and without limitation, a substituted aryl compound may have an aliphatic group coupled to the closed ring of the aryl base, such as with toluene. Solely by way of example and without limitation, a long-chain hydrocarbon may have a hydroxyl group bonded thereto.
Tautomers: Constitutional isomers of organic compounds that differ only in the position of the protons and electrons, and are interconvertible by migration of a hydrogen atom. Tautomers ordinarily exist together in equilibrium.
Therapeutically effective amount or dose: An amount sufficient to provide a beneficial, or therapeutic, effect to a subject or a given percentage of subjects.
Treating or treatment: With respect to disease, either term includes (1) preventing the disease, e.g., causing the clinical symptoms of the disease not to develop in an animal that may be exposed to or predisposed to the disease but does not yet experience or display symptoms of the disease, (2) inhibiting the disease, e.g., arresting or reducing the development of the disease or its clinical symptoms, or (3) relieving the disease, e.g., causing regression of the disease or its clinical symptoms.

### II. FLAVONES, QUINOLINONES, QUINAZOLINONES, AND AURONES

Flavones, quinolinones, quinazolinones, and aurones are disclosed. The disclosed flavones, quinolinones, and quinazolinones have a structure according to formula I, and the aurones have a structure according to formula II.

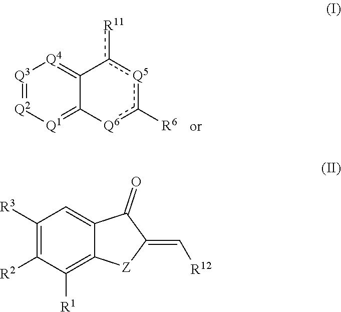

Pharmaceutically acceptable salts, hydrates, stereoisomers, and tautomers of the disclosed compounds also are encompassed by this disclosure.

In formula I, each bond represented by “ [symbol] ” is a single or double bond as needed to satisfy valence requirements; Q¹ is C—R¹ or N; Q² is C—R² or N; Q³ is C—R³ or N; Q⁴ is C—R⁴ or N; Q⁵ is C—R⁵, N, or N—R^{b}; Q⁶ is O, N, or N—R^{b}. In formula II, Z is O, S, or N—R^{b}. In formulas I and II, R¹ is X or H; R² is H or —OR^{a}; and R³ is X or H. In formula I, R⁴ and R⁵ are H.

In formula I, R⁶ is

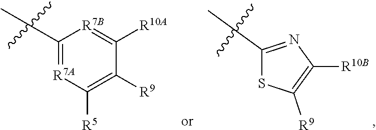

where R^{7A} and R^{7B} independently are N or CH, R⁸ is H or X, R⁹ is hydroxy, and R^{10A} and R^{10B} independently are X, —OR^{a}, C₁-C₅ aliphatic, substituted C₁-C₅ aliphatic, or cycloaliphatic. In formula I, R¹¹ is O, —OR^{a}, or N(R^{a})₂. In some aspects, when the compound has a structure according to formula I, at least one of Q¹-Q⁴ is N, or Q⁵ is N or N—R^{b}, or Q⁶ is N or N—R^{b}, or R¹ is other than O.

In formula II, R¹² is

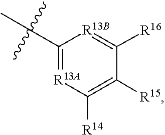

furan-2-yl, or furan-3-yl. R^{13A} and R^{13B} independently are N or CH; R¹⁴ is H, X, or —OR^{a}; R¹⁵ is —OR^{a}, H, cyano, tetrazolyl, or —CO₂R^{a}; and R¹⁶ is H, X, —OR^{a}, substituted aliphatic, cycloaliphatic, or alkynyl. Exemplary alkynyl groups include, but are not limited to, ethynyl and substituted ethynyl, wherein the ethynyl moiety is substituted with an aliphatic group, a heteroaliphatic group, a substituted or unsubstituted heteroaryl, or a substituted or unsubstituted aryl.

In any of the foregoing or following aspects, each R^{a} independently is H or C₁-C₅ aliphatic. Each R^{b} independently is H, C₁-C₅ aliphatic, or cycloaliphatic. X is halo. In some implementations, each X is independently Br or Cl.

In some aspects, R¹ and R³ are X, and R² is H or methoxy. In some aspects, R¹ is X, R² is methoxy or H, and R³ is H. In still other aspects, R¹ and R² are H, and R³ is X.

In any of the foregoing or following aspects, the compound may have a structure according to formula I, wherein Q⁵ is C—R⁵ (R⁵ is H), Q⁶ is O, and R¹¹ is O. In any of the foregoing or following aspects, the compound may have a structure according to formula I, wherein Q¹ is C—R¹ and R¹ is X, Q² is N or CH, Q³ is C—R³ and R³ is X, and Q⁴ is C—R⁴. In any of the foregoing or following aspects, the compound may have a structure according to formula I, wherein R⁶ is

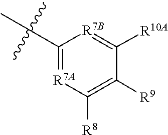

where (i) R⁹ is hydroxy; or (ii) R^{10A} and R^{10B} independently are Br, Cl, F, I, —CF₃, alkynyl, cyclopropyl, or —OCH₃; or (iii) both (i) and (ii).

In some aspects, the compound has a structure according to formula I-A or I-B:

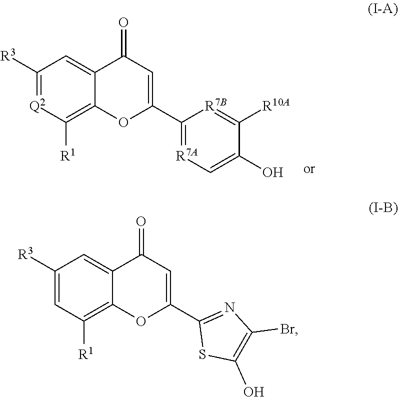

where R¹ is Br or Cl; R³ is Br, Cl, or H; Q², R^{7A}, and R^{7B} independently are N or CH; and R^{10A} is Br or Cl. In some implementations, the compound has a structure according to formula I-A, where Q², R^{7A}, and R^{7B} independently are N or CH, and (i) R¹ and R³ are Cl, and R^{10A} is Br or Cl, or (ii) R¹ and R³ are Br, and R^{10A} is Br or Cl, or (iii) R¹ is Br, R³ is Cl, and R^{10A} is Br or Cl, or (iv) R¹ is Br, R³ is H, and R^{10A} is Br or Cl. In some implementations, the compound has a structure according to formula I-B, where (i) R¹ and R³ are Cl, or (ii) R¹ and R³ are Br, or (iii) R¹ is Br and R³ is Cl, or (iv) R¹ is Br and R³ is H.

In some aspects, the compound has a structure according to formula I—C, I-D, or I-E:

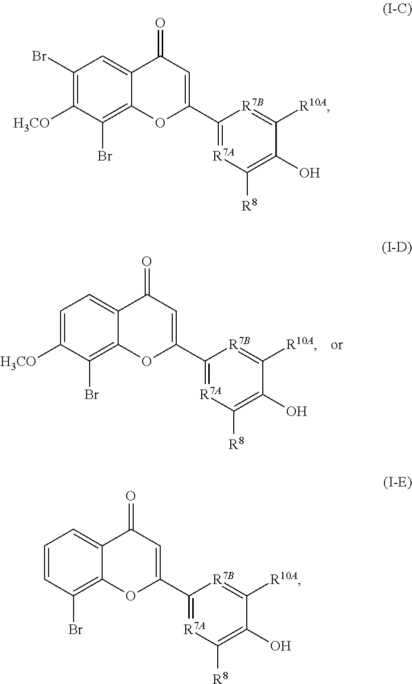

where R^{7A} and R^{7B} independently are N or CH; R⁸ is H, Cl, or F; and R^{10A} is Br, Cl, F, I, —CF₃, alkynyl, or cyclopropyl. In some implementations, the compound has a structure according to formula I-C or I-D, where (i) R^{7A} and R^{7B} independently are N or CH, R⁸ is H, and R^{10A} is Br, or (ii) R^{7A} and R^{7B} are CH, and R⁸ and R^{10A} are Cl, or (iii) R^{7A} and R^{7B} are CH, and R⁸ and R^{10A} are F, or (iv) R^{7A} and R^{7B} independently are N or CH, R⁸ is H, and R^{10A} is Cl, or (v) R^{7A} and R^{7B} are CH, R⁸ is H, and R^{10A} is I, or (vi) R^{7A} and R^{7B} are CH, R⁸ is H, and R^{10A} is —CF₃, or (vii) R^{7A} and R^{7B} independently are CH or N, R⁸ is H, and R^{10A} is alkynyl, or (viii) R^{7A} and R^{7B} independently are CH or N, R⁸ is H, and R^{10A} is cyclopropyl.

In some aspects, the compound has a structure according to formula I-F:

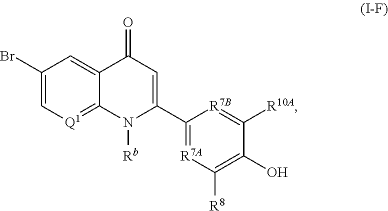

where Q¹ is N or C—Br; R^{b} is H, —CH₃, or cyclopropyl; R^{7A} and R^{7B} independently are N or CH; R⁸ is H, Cl, or F; and R^{10A} is Br, Cl, F, I, —CF₃, alkynyl, cyclopropyl, or —OCH₃. In some implementations, Q¹ is N or C—Br; R^{b} is H, —CH₃, or cyclopropyl, and (i) R^{7A} and R^{7B} independently are CH or N, R⁸ is H, and R^{10A} is Br, or (ii) R^{7A} and R^{7B} are CH, and R⁸ and R^{10A} are Cl, or (iii) R^{7A} and R^{7B} are CH, and R⁸ and R^{10A} are F, or (iv) R^{7A} and R^{7B} independently are CH or N, R⁸ is H, and R^{10A} is Cl, or (v) R^{7A} and R^{7B} are CH, R⁸ is H, and R^{10A} is I, or (vi) R^{7A} and R^{7B} are CH, R⁸ is H, and R^{10A} is —CF₃, or (vii) R^{7A} and R^{7B} independently are CH or N, R⁸ is H, and R^{10A} is alkynyl, or (viii) R^{7A} and R^{7B} independently are CH or N, R⁸ is H, and R^{10A} is cyclopropyl, or (ix) R^{7A} and R^{7B} independently are CH or N, R⁸ is H, and R^{10A} is —OCH₃.

In some aspects, the compound has a structure according to formula I-G:

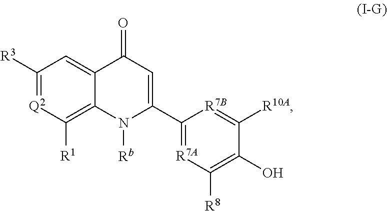

where Q², R^{7A} and R^{7B} independently are N or CH; R¹ is Cl or Br; R³ is Cl, Br, or H; R^{b} is H, —CH₃, or cyclopropyl; R⁸ is H, Cl, or F; and R^{10A} is Br, Cl, F, I, —CF₃, alkynyl, cyclopropyl, or —OCH₃. In some implementations, Q² is N or CH, R^{b} is H, —CH₃, or cyclopropyl, R¹ and R³ are Cl, and (i) R^{7A} and R^{7B} independently are CH or N, R¹ is H, and R^{10A} is Br, or (ii) R^{7A} and R^{7B} are CH, and R⁸ and R^{10A} are Cl, or (iii) R^{7A} and R^{7B} are CH, and R⁸ and R^{10A} are F, or (iv) R^{7A} and R^{7B} independently are CH or N, R⁸ is H, and R^{10A} is Cl, or (v) R^{7A} and R^{7B} are CH, R⁸ is H, and R^{10A} is I, or (vi) R^{7A} and R^{7B} are CH, R⁸ is H, and R^{OA} is —CF₃, or (vii) R^{7A} and R^{7B} independently are CH or N, R⁸ is H, and R^{10A} is alkynyl, or (viii) R^{7A} and R^{7B} independently are CH or N, R⁸ is H, and R^{10A} is cyclopropyl, or (ix) R^{7A} and R^{7B} independently are CH or N, R⁸ is H, and R^{10A} is —OCH₃. In some implementations, Q² is N or CH, R^{b} is H, —CH₃, or cyclopropyl, R¹ and R³ are Br, and (i) R^{7A} and R^{7B} independently are CH or N, R⁸ is H, and R^{10A} is Br, or (ii) R^{7A} and R^{7B} are CH, and R⁸ and R^{10A} are Cl, or (iii) R^{7A} and R^{7B} are CH, and R⁸ and R^{10A} are F, or (iv) R^{7A} and R^{7B} independently are CH or N, R⁸ is H, and R^{10A} is Cl, or (v) R^{7A} and R^{7B} are CH, R⁸ is H, and R^{10A} is I, or (vi) R^{7A} and R^{7B} are CH, R⁸ is H, and R^{10A} is —CF₃, or (vii) R^{7A} and R^{7B} independently are CH or N, R⁸ is H, and R^{10A} is alkynyl, or (viii) R^{7A} and R^{7B} independently are CH or N, R⁸ is H, and R^{10A} is cyclopropyl, or (ix) R^{7A} and R^{7B} independently are CH or N, R⁸ is H, and R^{10A} is —OCH₃. In some implementations Q² is N or CH, R^{b} is H, —CH₃, or cyclopropyl, R¹ is Br, and R³ is H, and (i) R^{7A} and R^{7B} independently are CH or N, R⁸ is H, and R^{10A} is Br, or (ii) R^{7A} and R^{7B} are CH, and R⁸ and R^{10A} are Cl, or (iii) R^{7A} and R^{7B} are CH, and R⁸ and R^{10A} are F, or (iv) R^{7A} and R^{7B} independently are CH or N, R⁸ is H, and R^{10A} is Cl, or (v) R^{7A} and R^{7B} are CH, R⁸ is H, and R^{10A} is I, or (vi) R^{7A} and R^{7B} are CH, R⁸ is H, and R^{10A} is —CF₃, or (vii) R^{7A} and R^{7B} independently are CH or N, R⁸ is H, and R^{10A} is alkynyl, or (viii) R^{7A} and R^{7B} independently are CH or N, R⁸ is H, and R^{10A} is cyclopropyl, or (ix) R^{7A} and R^{7B} independently are CH or N, R⁸ is H, and R^{10A} is —OCH₃. In some implementations Q² is N or CH, R^{b} is H, —CH₃, or cyclopropyl, R¹ is Br, and R³ is Cl, and (i) R^{7A} and R^{7B} independently are CH or N, R⁸ is H, and R^{10A} is Br, or (ii) R^{7A} and R^{7B} are CH, and R⁸ and R^{10A} are Cl, or (iii) R^{7A} and R^{7B} are CH, and R⁸ and R^{10A} are F, or (iv) R^{7A} and R^{7B} independently are CH or N, R⁸ is H, and R^{10A} is Cl, or (v) R^{7A} and R^{7B} are CH, R^{S} is H, and R^{10A} is I, or (vi) R^{7A} and R^{7B} are CH, R⁸ is H, and R^{10A} is —CF₃, or (vii) R^{7A} and R^{7B} independently are CH or N, R³ is H, and R^{10A} is alkynyl, or (viii) R^{7A} and R^{7B} independently are CH or N, R⁸ is H, and R^{10A} is cyclopropyl, or (ix) R^{7A} and R^{7B} independently are CH or N, R⁸ is H, and R^{10A} is —OCH₃.

In some aspects, the compound has a structure according to formula I-H:

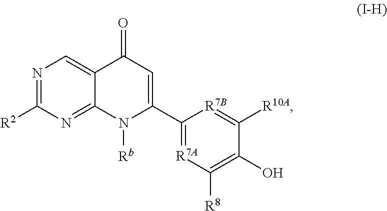

where R² is H or —OCH₃; R^{b} is H, —CH₃, or cyclopropyl; R^{7A} and R^{7B} independently are N or CH; R⁸ is H, Cl, or F; and R^{10A} is Br, Cl, F, I, —CF₃, alkynyl, cyclopropyl, or —OCH₃. In some implementations, R² is H or —OCH₃; R^{b} is H, —CH₃, or cyclopropyl, and (i) R^{7A} and R^{7B} independently are CH or N, R⁸ is H, and R^{10A} is Br, or (ii) R^{7A} and R^{7B} are CH, and R⁸ and R^{10A} are Cl, or (iii) R^{7A} and R^{7R} are CH, and R⁸ and R^{10A} are F, or (iv) R^{7A} and R^{7B} independently are CH or N, R⁸ is H, and R^{10A} is Cl, or (v) R^{7A} and R^{7B} are CH, R⁸ is H, and R^{10A} is I, or (vi) R^{7A} and R^{7B} are CH, R⁸ is H, and R^{10A} is —CF₃, or (vii) R^{7A} and R^{7B} independently are CH or N, R¹ is H, and R^{10A} is alkynyl, or (viii) R^{7A} and R^{7B} independently are CH or N, R⁸ is H, and R^{10A} is cyclopropyl, or (ix) R^{7A} and R^{7B} independently are CH or N, R⁸ is H, and R^{10A} is —OCH₃.

In some aspects, the compound has a structure according to any one of formulas I-I to I-Q:

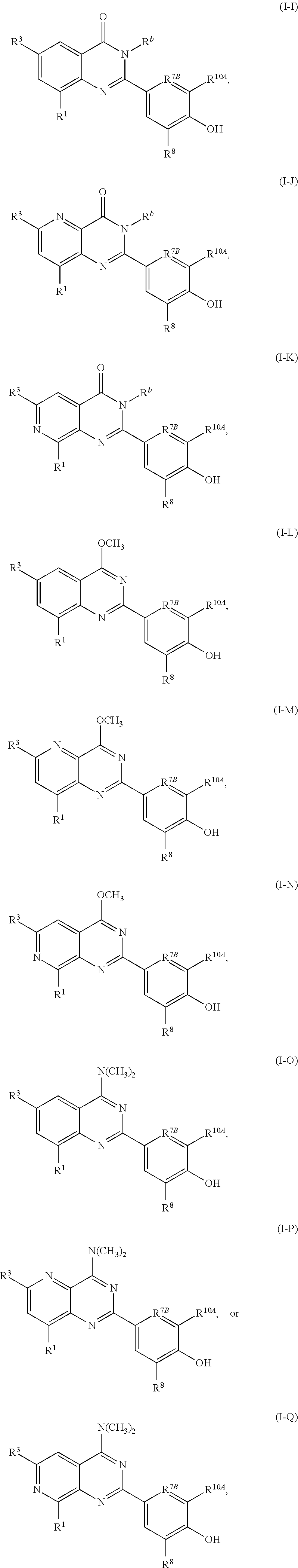

where R¹ and R³ independently are Cl, Br, or H; R^{b} is H, —CH₃, or cyclopropyl; R^{7B} is CH or N; R⁸ is H, Cl, or F; and R^{10A} is Br, Cl, F, I, —CF₃, alkynyl, cyclopropyl, or —OCH₃. In some implementations, the compound has a structure according to formula I-I, I-J, or I-K, where R^{b} is H, —CH₃, or cyclopropyl, R¹ and R³ are Cl, and (i) R^{7B} is CH or N, R¹ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to formula I-I, I-J, or I-K, where R^{b} is H, —CH₃, or cyclopropyl, R¹ and R³ are Br, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to formula I-I, I-J, or I-K, where R^{b} is H, —CH₃, or cyclopropyl, R¹ is Br, R³ is Cl, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to formula I-I, I-J, or I-K, where R^{b} is H, —CH₃, or cyclopropyl, R¹ is Cl, R³ is H, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to formula I-I, I-J, or I-K, where R^{b} is H, —CH₃, or cyclopropyl, R¹ is H, R³ is Cl, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to formula I-I, I-J, or I-K, where R^{b} is H, —CH₃, or cyclopropyl, R¹ is H, R³ is Br, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to formula I-I, I-J, or I-K, where R^{b} is H, —CH₃, or cyclopropyl, R¹ is Br, R³ is H, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃.

In some implementations, the compound has a structure according to any one of formulas I-L to I-Q, where R¹ and R³ are Cl, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to any one of formulas I-L to I-Q, where R¹ and R³ are Br, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to any one of formulas I-L to I-Q, where R¹ is Br, R³ is Cl, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to any one of formulas I-L to I-Q, where Rⁱ is Cl, R³ is H, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R¹CA is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to any one of formulas I-L to I-Q, where R¹ is H, R³ is Cl, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to any one of formulas I-L to I-Q, where R¹ is H, R³ is Br, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and RiOA are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃. In some implementations, the compound has a structure according to any one of formulas I-L to I-Q, where R¹ is Br, R³ is H, and (i) R^{7B} is CH or N, R⁸ is H, and R^{10A} is —CF₃, or (ii) R^{7B} is CH or N, R⁸ is H, and R^{10A} is Cl, or (iii) R^{7B} is CH or N, R⁸ is H, and R^{10A} is —OCH₃, or (iv) R^{7B} is CH or N, R⁸ is H, and R^{10A} is Br, or (v) R^{7B} is CH, and R⁸ and R^{10A} are Cl, or (vi) R^{7B} is CH, and R⁸ and R^{10A} are F, or (vii) R^{7B} is CH or N, R⁸ is H, and R^{10A} is alkynyl, or (viii) R^{7B} is CH or N, R⁸ is H, and R^{10A} is cyclopropyl, or (ix) R^{7B} is CH or N, R⁸ is H, and R^{10A} is —OCH₃.

In some aspects, the compound has a structure according to formula II-A

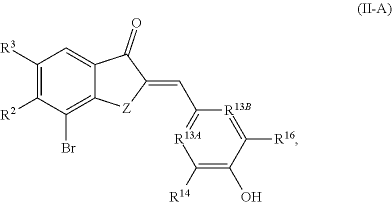

where Z is O, S, N(H), N—CH₃, or N-cyclopropyl; R² is H or —OCH₃; R³ is H or Br; R^{13A} and R^{13B} independently are CH or N; R¹⁴ is H, Cl, or F; and R¹⁶ is Br, Cl, F, I, —CF₃, alkynyl, cyclopropyl, or —OCH₃. In some implementations, R² is H or —OCH₃, R³ is H or Br, Z is O, and (i) R^{13A} and R^{13B} independently are CH or N, R¹⁴ is H, and R¹⁶ is Br, or (ii) R^{13A} and R^{13B} are CH, and R¹⁴ and R¹⁶ are Cl, or (iii) R^{13A} and R^{13B} are CH, and R¹⁴ and R¹⁶ are F, or (iv) R^{13A} and R^{13B} independently are CH or N, R¹⁴ is H, and R¹⁶ is Cl, or (v) R^{13A} and R^{13B} are CH, R¹⁴ is H, and R¹⁶ is I, or (vi) R^{13A} and R^{13B} are CH, R¹⁴ is H, and R¹⁶ is —CF₃, or (vii) R^{13A} and R^{13B} independently are CH or N, R¹⁴ is H, and R¹⁶ is alkynyl, or (viii) R^{13A} and R^{13B} independently are CH or N, R¹⁴ is H, and R¹⁶ is cyclopropyl, or (ix) R^{13A} and R^{13B} independently are CH or N, R¹⁴ is H, and R¹⁶ is —OCH₃. In some implementations, R² is H or —OCH₃, R³ is H or Br, Z is S, and (i) R^{13A} and R^{13B} independently are CH or N, R¹⁴ is H, and R¹⁶ is Br, or (ii) R^{13A} and R^{13B} are CH, and R¹⁴ and R¹⁶ are Cl, or (iii) R^{13A} and R^{13B} are CH, and R¹⁴ and R¹⁶ are F, or (iv) R^{13A} and R^{13B} independently are CH or N, R¹⁴ is H, and R¹⁶ is Cl, or (v) R^{13A} and R^{13B} are CH, R¹⁴ is H, and R¹⁶ is I, or (vi) R^{13A} and R^{13B} are CH, R¹⁴ is H, and R¹⁶ is —CF₃, or (vii) R^{13A} and R^{13B} independently are CH or N, R¹⁴ is H, and R¹⁶ is alkynyl, or (viii) R^{13A} and R^{13B} independently are CH or N, R¹⁴ is H, and R¹⁶ is cyclopropyl, or (ix) R^{13A} and R^{13B} independently are CH or N, R¹⁴ is H, and R¹⁶ is —OCH₃. In some implementations, R² is H or —OCH₃, R³ is H or Br, Z is NH, and (i) R^{13A} and R^{13B} independently are CH or N, R¹⁴ is H, and R¹⁶ is Br, or (ii) R^{13A} and R^{13B} are CH, and R¹⁴ and R¹⁶ are Cl, or (iii) R^{13A} and R^{13B} are CH, and R¹⁴ and R¹⁶ are F, or (iv) R^{13A} and R^{13B} independently are CH or N, R¹⁴ is H, and R¹⁶ is Cl, or (v) R^{13A} and R^{13B} are CH, R¹⁴ is H, and R¹⁶ is I, or (vi) R^{13A} and R^{13B} are CH, R¹⁴ is H, and R¹⁶ is —CF₃, or (vii) R^{13A} and R^{13B} independently are CH or N, R¹⁴ is H, and R¹⁶ is alkynyl, or (viii) R^{13A} and R^{13B} independently are CH or N, R¹⁴ is H, and R¹⁶ is cyclopropyl, or (ix) R^{13A} and R^{13B} independently are CH or N, R¹⁴ is H, and R¹⁶ is —OCH₃. In some implementations, R² is H or —OCH₃, R³ is H or Br, Z is N-cyclopropyl, and (i) R^{13A} and R^{13B} independently are CH or N, R¹⁴ is H, and R¹⁶ is Br, or (ii) R^{13A} and R^{13B} are CH, and R¹⁴ and R¹⁶ are Cl, or (iii) R^{13A} and R^{13B} are CH, and R¹⁴ and R¹⁶ are F, or (iv) R^{13A} and R^{13B} independently are CH or N, R¹⁴ is H, and R¹⁶ is Cl, or (v) R^{13A} and R^{13B} are CH, R¹⁴ is H, and R¹⁶ is I, or (vi) R^{13A} and R^{13B} are CH, R¹⁴ is H, and R¹⁶ is —CF₃, or (vii) R^{13A} and R^{13B} independently are CH or N, R¹⁴ is H, and R¹⁶ is alkynyl, or (viii) R^{13A} and R^{13B} independently are CH or N, R¹⁴ is H, and R¹⁶ is cyclopropyl, or (ix) R^{13A} and R^{13B} independently are CH or N, R¹⁴ is H, and R¹⁶ is —OCH₃.

Several exemplary compounds are shown in Table 1.

TABLE 1

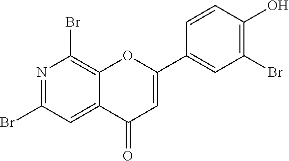 [SMILES: O=c1cc(-c2ccc(O)c(Br)c2)oc2c(Br)nc(Br)cc12]

JKT.909A

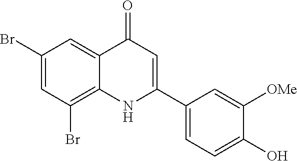 [SMILES: COc1cc(-c2cc(=O)c3cc(Br)cc(Br)c3[nH]2)ccc1O]

JKT.736A

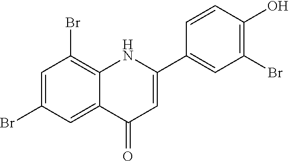 [SMILES: O=c1cc(-c2ccc(O)c(Br)c2)[nH]c2c(Br)cc(Br)cc12]

JKT.944A

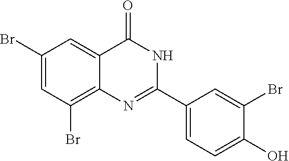 [SMILES: O=c1[nH]c(-c2ccc(O)c(Br)c2)nc2c(Br)cc(Br)cc12]

JKT.734C

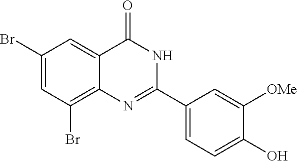 [SMILES: COc1cc(-c2nc3c(Br)cc(Br)cc3c(=O)[nH]2)ccc1O]

JKT.737B

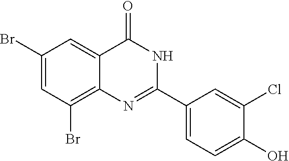 [SMILES: O=c1[nH]c(-c2ccc(O)c(Cl)c2)nc2c(Br)cc(Br)cc12]

JKT.772A

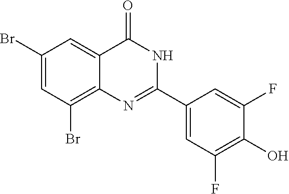 [SMILES: O=c1[nH]c(-c2cc(F)c(O)c(F)c2)nc2c(Br)cc(Br)cc12]

JKT.774A

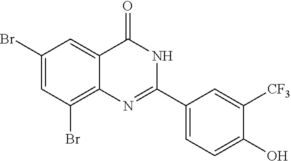 [SMILES: O=c1[nH]c(-c2ccc(O)c(C(F)(F)F)c2)nc2c(Br)cc(Br)cc12]

JKT.775A

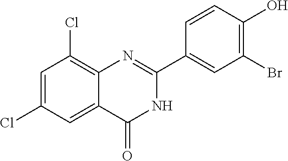 [SMILES: O=c1[nH]c(-c2ccc(O)c(Br)c2)nc2c(Cl)cc(Cl)cc12]

JKT.805A

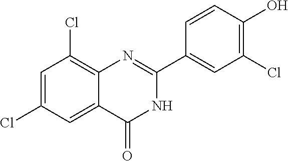 [SMILES: O=c1[nH]c(-c2ccc(O)c(Cl)c2)nc2c(Cl)cc(Cl)cc12]

JKT.806A

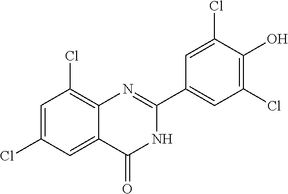 [SMILES: O=c1[nH]c(-c2cc(Cl)c(O)c(Cl)c2)nc2c(Cl)cc(Cl)cc12]

JKT.807A

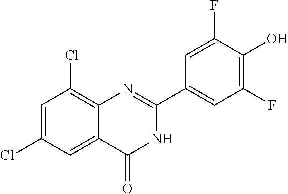 [SMILES: O=c1[nH]c(-c2cc(F)c(O)c(F)c2)nc2c(Cl)cc(Cl)cc12]

JKT.808A

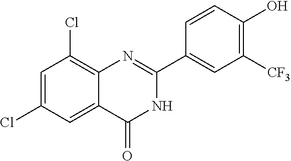 [SMILES: O=c1[nH]c(-c2ccc(O)c(C(F)(F)F)c2)nc2c(Cl)cc(Cl)cc12]

JKT.809A

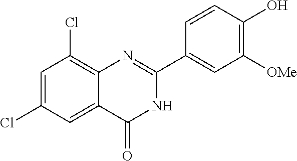 [SMILES: COc1cc(-c2nc3c(Cl)cc(Cl)cc3c(=O)[nH]2)ccc1O]

JKT.810A

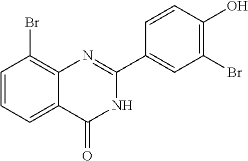 [SMILES: O=c1[nH]c(-c2ccc(O)c(Br)c2)nc2c(Br)cccc12]

JKT.827A

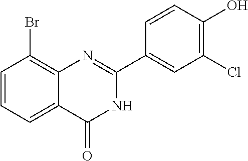 [SMILES: O=c1[nH]c(-c2ccc(O)c(Cl)c2)nc2c(Br)cccc12]

JKT.828A

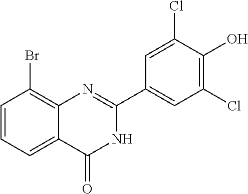 [SMILES: O=c1[nH]c(-c2cc(Cl)c(O)c(Cl)c2)nc2c(Br)cccc12]

JKT.829A

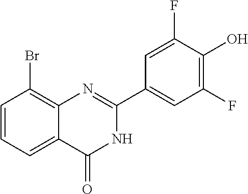 [SMILES: O=c1[nH]c(-c2cc(F)c(O)c(F)c2)nc2c(Br)cccc12]

JKT.830A

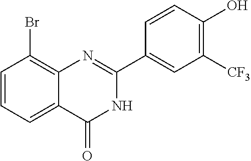 [SMILES: O=c1[nH]c(-c2ccc(O)c(C(F)(F)F)c2)nc2c(Br)cccc12]

JKT.831A

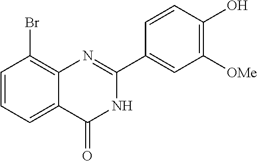 [SMILES: COc1cc(-c2nc3c(Br)cccc3c(=O)[nH]2)ccc1O]

JKT.832A

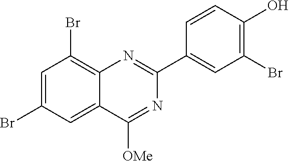 [SMILES: COc1nc(-c2ccc(O)c(Br)c2)nc2c(Br)cc(Br)cc12]

JKT.842A

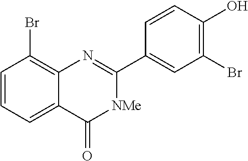 [SMILES: Cn1c(-c2ccc(O)c(Br)c2)nc2c(Br)cccc2c1=O]

JKT.866A

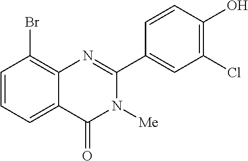 [SMILES: Cn1c(-c2ccc(O)c(Cl)c2)nc2c(Br)cccc2c1=O]

JKT.870A

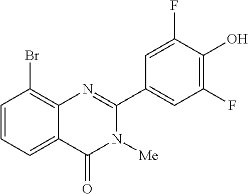 [SMILES: Cn1c(-c2cc(F)c(O)c(F)c2)nc2c(Br)cccc2c1=O]

JKT.873A

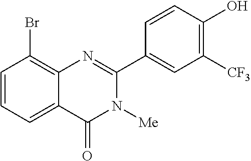 [SMILES: Cn1c(-c2ccc(O)c(C(F)(F)F)c2)nc2c(Br)cccc2c1=O]

JKT.875A

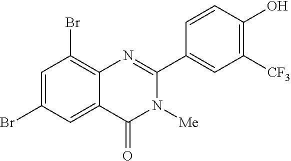 [SMILES: Cn1c(-c2ccc(O)c(C(F)(F)F)c2)nc2c(Br)cc(Br)cc2c1=O]

JKT.874D

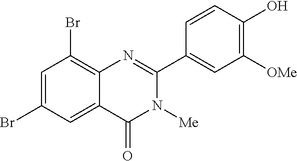 [SMILES: COc1cc(-c2nc3c(Br)cc(Br)cc3c(=O)n2C)ccc1O]

JKT.876A

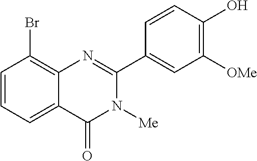 [SMILES: COc1cc(-c2nc3c(Br)cccc3c(=O)n2C)ccc1O]

JKT.877A

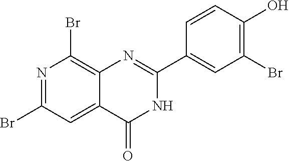 [SMILES: O=c1[nH]c(-c2ccc(O)c(Br)c2)nc2c(Br)nc(Br)cc12]

JKT.880B

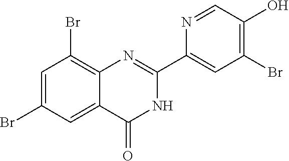 [SMILES: O=c1[nH]c(-c2cc(Br)c(O)cn2)nc2c(Br)cc(Br)cc12]

JKT.932F

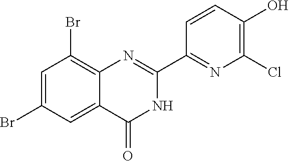 [SMILES: O=c1[nH]c(-c2ccc(O)c(Cl)n2)nc2c(Br)cc(Br)cc12]

JKT.956A

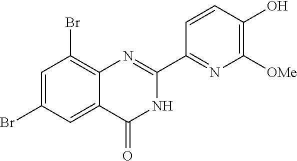 [SMILES: COc1nc(-c2nc3c(Br)cc(Br)cc3c(=O)[nH]2)ccc1O]

JKT.968C

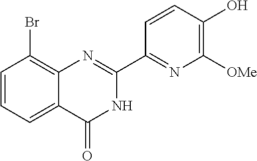 [SMILES: COc1nc(-c2nc3c(Br)cccc3c(=O)[nH]2)ccc1O]

JKT.975A

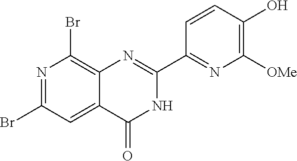 [SMILES: COc1nc(-c2nc3c(Br)nc(Br)cc3c(=O)[nH]2)ccc1O]

JKT.976B

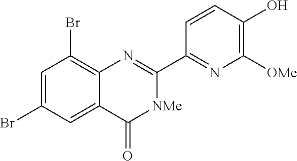 [SMILES: COc1nc(-c2nc3c(Br)cc(Br)cc3c(=O)n2C)ccc1O]

JKT.983A

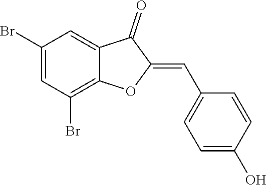 [SMILES: O=C1/C(=C/c2ccc(O)cc2)Oc2c(Br)cc(Br)cc21]

JKT.629A

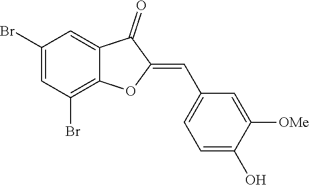 [SMILES: COc1cc(/C=C2\Oc3c(Br)cc(Br)cc3C2=O)ccc1O]

JKT.630A

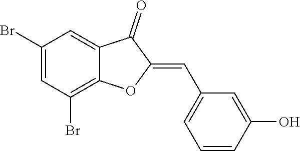 [SMILES: O=C1/C(=C/c2cccc(O)c2)Oc2c(Br)cc(Br)cc21]

JKT.631A

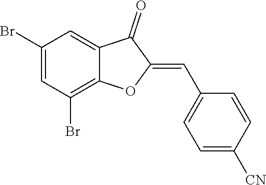 [SMILES: N#Cc1ccc(/C=C2\Oc3c(Br)cc(Br)cc3C2=O)cc1]

JKT.632A

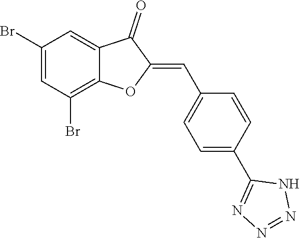 [SMILES: O=C1/C(=C/c2ccc(-c3nnn[nH]3)cc2)Oc2c(Br)cc(Br)cc21]

JKT.633A

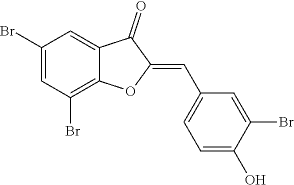 [SMILES: O=C1/C(=C/c2ccc(O)c(Br)c2)Oc2c(Br)cc(Br)cc21]

JKT.634A

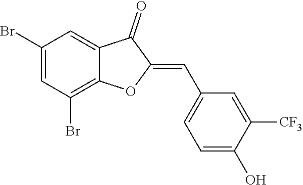 [SMILES: O=C1/C(=C/c2ccc(O)c(C(F)(F)F)c2)Oc2c(Br)cc(Br)cc21]

JKT.635A

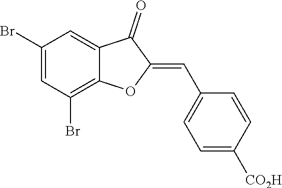 [SMILES: O=C(O)c1ccc(/C=C2\Oc3c(Br)cc(Br)cc3C2=O)cc1]

JKT.636A

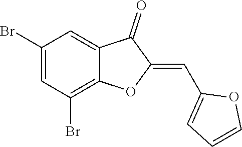 [SMILES: O=C1/C(=C/c2ccco2)Oc2c(Br)cc(Br)cc21]

JKT.637A

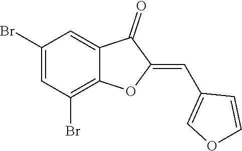 [SMILES: O=C1/C(=C/c2ccoc2)Oc2c(Br)cc(Br)cc21]

JKT.638A

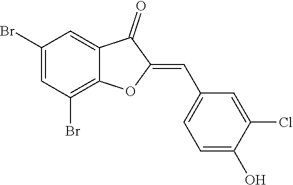 [SMILES: O=C1/C(=C/c2ccc(O)c(Cl)c2)Oc2c(Br)cc(Br)cc21]

JKT.726A

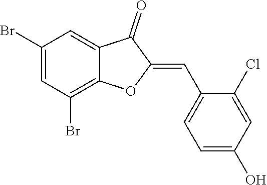 [SMILES: O=C1/C(=C/c2ccc(O)cc2Cl)Oc2c(Br)cc(Br)cc21]

JKT.727A

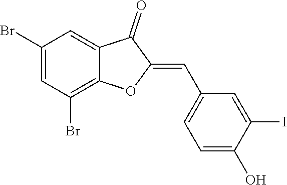 [SMILES: O=C1/C(=C/c2ccc(O)c(I)c2)Oc2c(Br)cc(Br)cc21]

JKT.728A

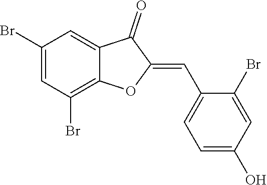 [SMILES: O=C1/C(=C/c2ccc(O)cc2Br)Oc2c(Br)cc(Br)cc21]

JKT.758A

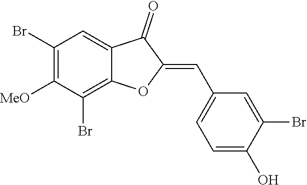 [SMILES: COc1c(Br)cc2c(c1Br)O/C(=C\c1ccc(O)c(Br)c1)C2=O]

JKT.759A

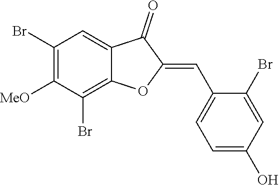 [SMILES: COc1c(Br)cc2c(c1Br)O/C(=C\c1ccc(O)cc1Br)C2=O]

JKT.760A

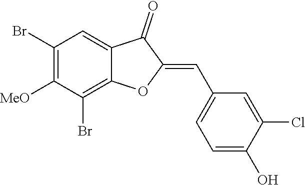 [SMILES: COc1c(Br)cc2c(c1Br)O/C(=C\c1ccc(O)c(Cl)c1)C2=O]

JKT.761A

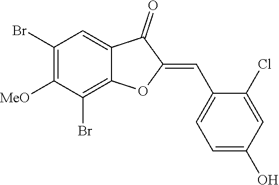 [SMILES: COc1c(Br)cc2c(c1Br)O/C(=C\c1ccc(O)cc1Cl)C2=O]

JKT.762A

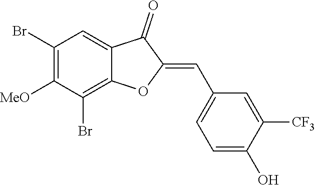 [SMILES: COc1c(Br)cc2c(c1Br)O/C(=C\c1ccc(O)c(C(F)(F)F)c1)C2=O]

JKT.763A

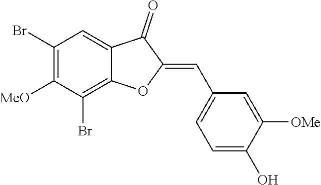 [SMILES: COc1cc(/C=C2\Oc3c(cc(Br)c(OC)c3Br)C2=O)ccc1O]

JKT.764A

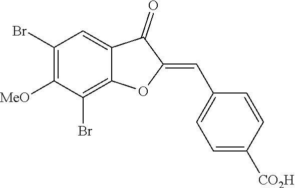 [SMILES: COc1c(Br)cc2c(c1Br)O/C(=C\c1ccc(C(=O)O)cc1)C2=O]

JKT.765A

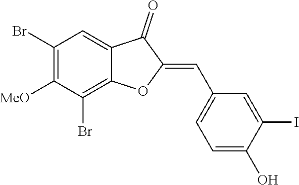 [SMILES: COc1c(Br)cc2c(c1Br)O/C(=C\c1ccc(O)c(I)c1)C2=O]

JKT.813A

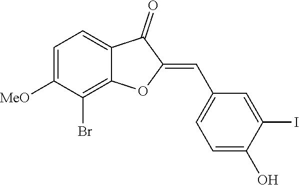 [SMILES: COc1ccc2c(c1Br)O/C(=C\c1ccc(O)c(I)c1)C2=O]

JKT.814A

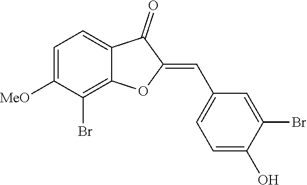 [SMILES: COc1ccc2c(c1Br)O/C(=C\c1ccc(O)c(Br)c1)C2=O]

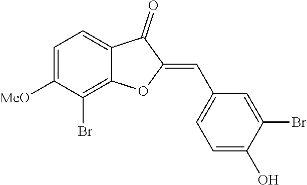 [SMILES: COc1ccc2c(c1Br)O/C(=C\c1ccc(O)c(Br)c1)C2=O]

JKT.815A

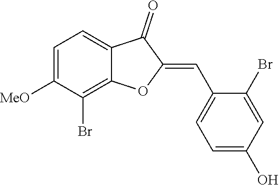 [SMILES: COc1ccc2c(c1Br)O/C(=C\c1ccc(O)cc1Br)C2=O]

JKT.816A

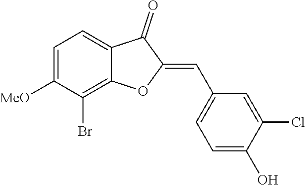 [SMILES: COc1ccc2c(c1Br)O/C(=C\c1ccc(O)c(Cl)c1)C2=O]

JKT.817A

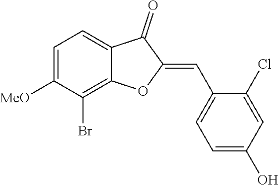 [SMILES: COc1ccc2c(c1Br)O/C(=C\c1ccc(O)cc1Cl)C2=O]

JKT.818A

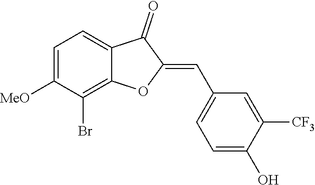 [SMILES: COc1ccc2c(c1Br)O/C(=C\c1ccc(O)c(C(F)(F)F)c1)C2=O]

JKT.819A

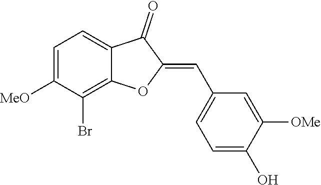 [SMILES: COc1cc(/C=C2\Oc3c(ccc(OC)c3Br)C2=O)ccc1O]

JKT.820A

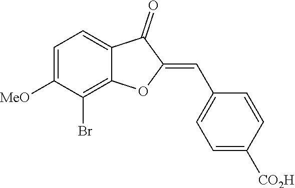 [SMILES: COc1ccc2c(c1Br)O/C(=C\c1ccc(C(=O)O)cc1)C2=O]

JKT.821A

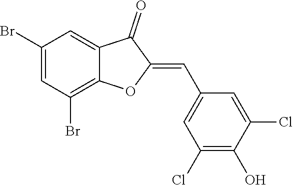 [SMILES: O=C1/C(=C/c2cc(Cl)c(O)c(Cl)c2)Oc2c(Br)cc(Br)cc21]

JKT.822A

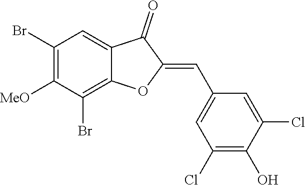 [SMILES: COc1c(Br)cc2c(c1Br)O/C(=C\c1cc(Cl)c(O)c(Cl)c1)C2=O]

JKT.823A

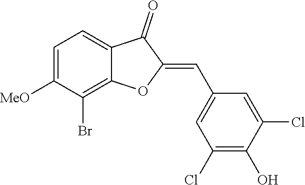 [SMILES: COc1ccc2c(c1Br)O/C(=C\c1cc(Cl)c(O)c(Cl)c1)C2=O]

JKT.824A

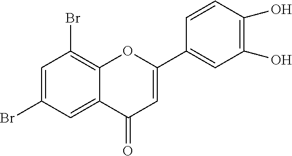 [SMILES: O=c1cc(-c2ccc(O)c(O)c2)oc2c(Br)cc(Br)cc12]

1004A

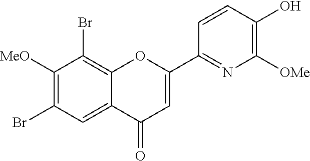 [SMILES: COc1nc(-c2cc(=O)c3cc(Br)c(OC)c(Br)c3o2)ccc1O]

1041A

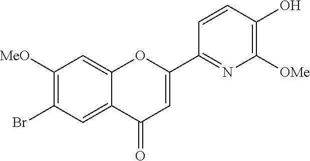 [SMILES: COc1cc2oc(-c3ccc(O)c(OC)n3)cc(=O)c2cc1Br]

1058A

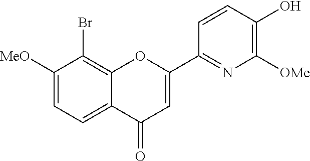 [SMILES: COc1ccc2c(=O)cc(-c3ccc(O)c(OC)n3)oc2c1Br]

1059A

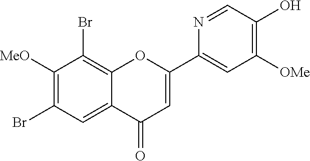 [SMILES: COc1cc(-c2cc(=O)c3cc(Br)c(OC)c(Br)c3o2)ncc1O]

1040A

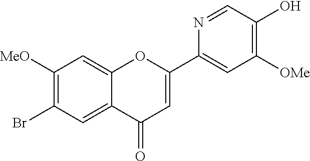 [SMILES: COc1cc(-c2cc(=O)c3cc(Br)c(OC)cc3o2)ncc1O]

1063B

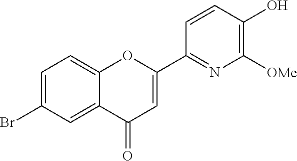 [SMILES: COc1nc(-c2cc(=O)c3cc(Br)ccc3o2)ccc1O]

1064A

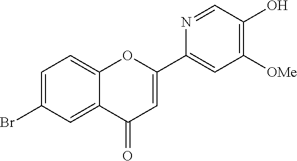 [SMILES: COc1cc(-c2cc(=O)c3cc(Br)ccc3o2)ncc1O]

1065A

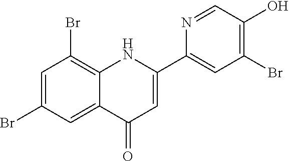 [SMILES: O=c1cc(-c2cc(Br)c(O)cn2)[nH]c2c(Br)cc(Br)cc12]

1066A

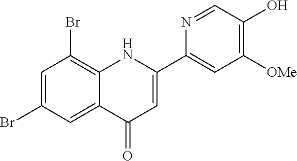 [SMILES: COc1cc(-c2cc(=O)c3cc(Br)cc(Br)c3[nH]2)ncc1O]

1077A

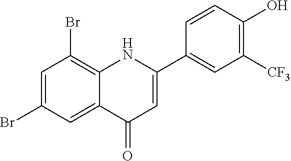 [SMILES: O=c1cc(-c2ccc(O)c(C(F)(F)F)c2)[nH]c2c(Br)cc(Br)cc12]

1095B

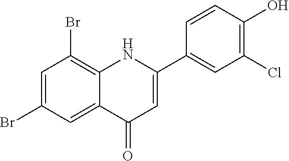 [SMILES: O=c1cc(-c2ccc(O)c(Cl)c2)[nH]c2c(Br)cc(Br)cc12]

1103A

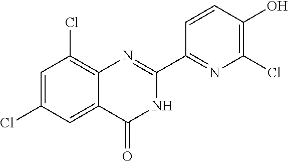 [SMILES: O=c1[nH]c(-c2ccc(O)c(Cl)n2)nc2c(Cl)cc(Cl)cc12]

963A

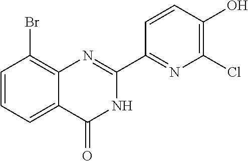 [SMILES: O=c1[nH]c(-c2ccc(O)c(Cl)n2)nc2c(Br)cccc12]

964A

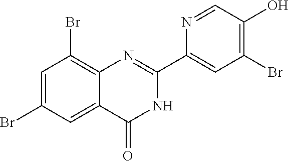 [SMILES: O=c1[nH]c(-c2cc(Br)c(O)cn2)nc2c(Br)cc(Br)cc12]

1005A

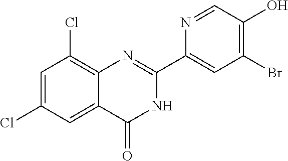 [SMILES: O=c1[nH]c(-c2cc(Br)c(O)cn2)nc2c(Cl)cc(Cl)cc12]

1006A

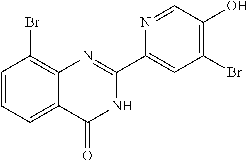 [SMILES: O=c1[nH]c(-c2cc(Br)c(O)cn2)nc2c(Br)cccc12]

1007A

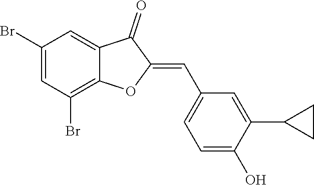 [SMILES: O=C1/C(=C/c2ccc(O)c(C3CC3)c2)Oc2c(Br)cc(Br)cc21]

1131A

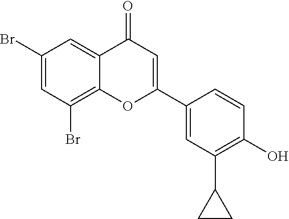 [SMILES: O=c1cc(-c2ccc(O)c(C3CC3)c2)oc2c(Br)cc(Br)cc12]

1132A

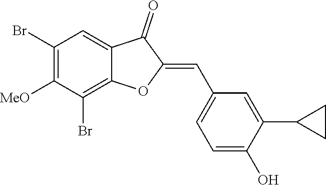 [SMILES: COc1c(Br)cc2c(c1Br)O/C(=C\c1ccc(O)c(C3CC3)c1)C2=O]

1134A

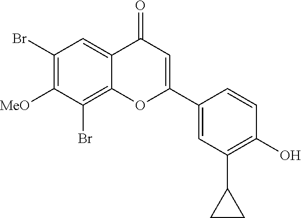 [SMILES: COc1c(Br)cc2c(=O)cc(-c3ccc(O)c(C4CC4)c3)oc2c1Br]

1140A

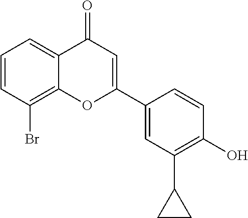 [SMILES: O=c1cc(-c2ccc(O)c(C3CC3)c2)oc2c(Br)cccc12]

1141A

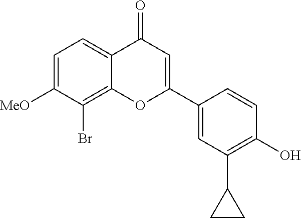 [SMILES: COc1ccc2c(=O)cc(-c3ccc(O)c(C4CC4)c3)oc2c1Br]

1142A

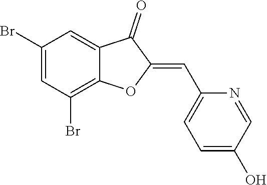 [SMILES: O=C1/C(=C/c2ccc(O)cn2)Oc2c(Br)cc(Br)cc21]

1143A

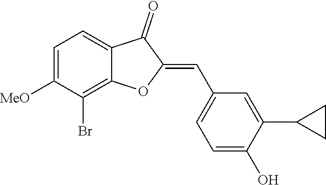 [SMILES: COc1ccc2c(c1Br)O/C(=C\c1ccc(O)c(C3CC3)c1)C2=O]

1135B

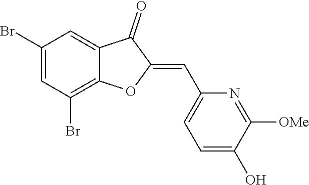 [SMILES: COc1nc(/C=C2\Oc3c(Br)cc(Br)cc3C2=O)ccc1O]

1150A

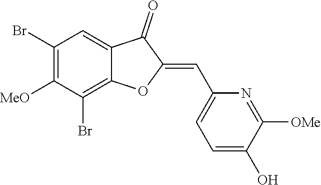 [SMILES: COc1nc(/C=C2\Oc3c(cc(Br)c(OC)c3Br)C2=O)ccc1O]

1159A

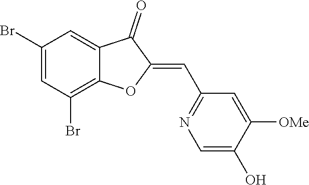 [SMILES: COc1cc(/C=C2\Oc3c(Br)cc(Br)cc3C2=O)ncc1O]

1163A

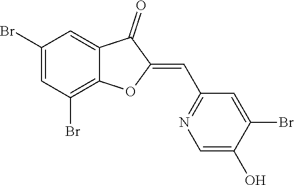 [SMILES: O=C1/C(=C/c2cc(Br)c(O)cn2)Oc2c(Br)cc(Br)cc21]

1173A

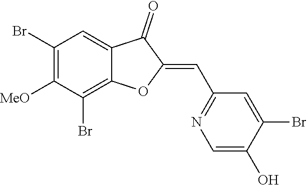 [SMILES: COc1c(Br)cc2c(c1Br)O/C(=C\c1cc(Br)c(O)cn1)C2=O]

1174A

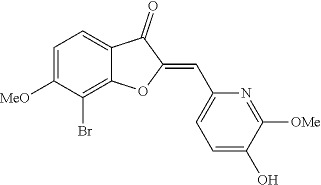 [SMILES: COc1ccc2c(c1Br)O/C(=C\c1ccc(O)c(OC)n1)C2=O]

1188A

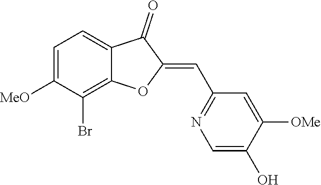 [SMILES: COc1cc(/C=C2\Oc3c(ccc(OC)c3Br)C2=O)ncc1O]

1189A

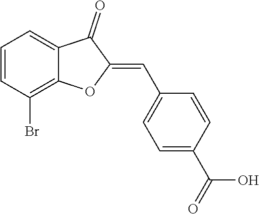 [SMILES: O=C(O)c1ccc(/C=C2\Oc3c(Br)cccc3C2=O)cc1]

1210A

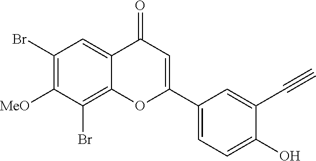 [SMILES: C#Cc1cc(-c2cc(=O)c3cc(Br)c(OC)c(Br)c3o2)ccc1O]

1212A

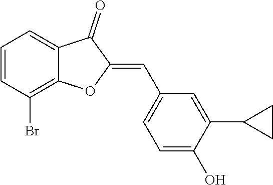 [SMILES: O=C1/C(=C/c2ccc(O)c(C3CC3)c2)Oc2c(Br)cccc21]

1220A

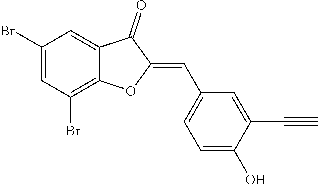 [SMILES: C#Cc1cc(/C=C2\Oc3c(Br)cc(Br)cc3C2=O)ccc1O]

1224A

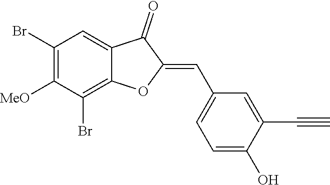 [SMILES: C#Cc1cc(/C=C2\Oc3c(cc(Br)c(OC)c3Br)C2=O)ccc1O]

1229A

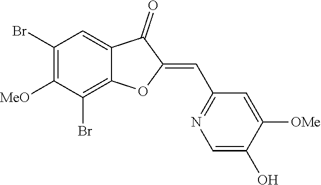 [SMILES: COc1cc(/C=C2\Oc3c(cc(Br)c(OC)c3Br)C2=O)ncc1O]

1164A

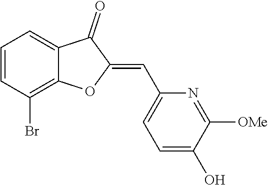 [SMILES: COc1nc(/C=C2\Oc3c(Br)cccc3C2=O)ccc1O]

1226A

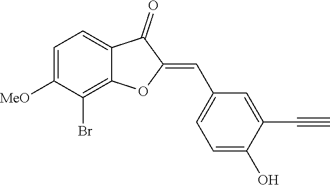 [SMILES: C#Cc1cc(/C=C2\Oc3c(ccc(OC)c3Br)C2=O)ccc1O]

1231A

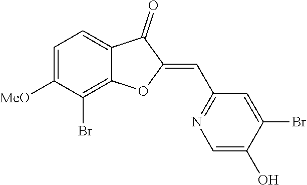 [SMILES: COc1ccc2c(c1Br)O/C(=C\c1cc(Br)c(O)cn1)C2=O]

1190B

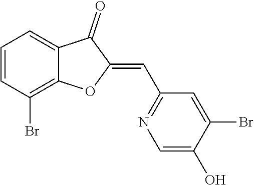 [SMILES: O=C1/C(=C/c2cc(Br)c(O)cn2)Oc2c(Br)cccc21]

1237A

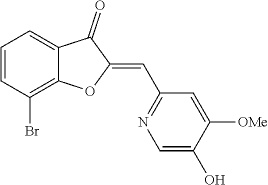 [SMILES: COc1cc(/C=C2\Oc3c(Br)cccc3C2=O)ncc1O]

1236B

### III. PHARMACEUTICAL COMPOSITIONS

The disclosure also encompasses pharmaceutical compositions comprising one or more of the disclosed compounds. A pharmaceutical composition comprises a compound as disclosed herein and a pharmaceutically acceptable excipient.

The compounds described herein can be used to prepare therapeutic pharmaceutical compositions. The compounds may be added to the compositions in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartrate, succinate, benzoate, ascorbate, α-ketoglutarate, and b-glycerophosphate. Suitable inorganic salts may also be formed, including hydrochloride, halide, sulfate, nitrate, bicarbonate, and carbonate salts.

Pharmaceutically acceptable salts may be obtained using procedures known to persons of ordinary skill in the art, for example by reacting a sufficiently basic compound, such as an amine, with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human or veterinary patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 2% to about 60% of the weight of a given unit dosage form. The amount of active compound in such therapeutically useful compositions is such that an effective dosage level can be obtained.

The tablets, troches, pills, capsules, and the like may also contain one or more of the following excipients: binders such as gum tragacanth, acacia, cornstarch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, methods of preparation can include vacuum drying and freeze drying techniques, which yield a powder of the active ingredient plus any additional desired ingredient present in the previously sterile-filtered solutions.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

### IV. METHODS OF USE

Casein kinase 2 (CK2) is a potent driver of inflammation, such as neuroinflammation. CK2, primarily isoform CK2A2, is a potent driver of glial inflammation, especially in Alzheimer's disease (AD), and pharmacological inhibition or genetic perturbation of CK2 reduces biochemical and transcriptional programs driving inflammation in glia. NF-κB is a major transcriptional driver of inflammation. CK2 modulates NF-κB activity via phosphorylation of NF-κB S529 and IκBα S32 and downregulates NF-κB transcriptional signatures. Isoform CK2A2 specifically phosphorylates AKTI S129 (FIG. 13). CK2 activity is upregulated in AD, Huntington's disease (HD), and Parkinson's disease (PD) patients in part through CK2A2 upregulation at the transcript and protein level or post-translational modification (such as, but not limited to, S-nitrosylation, phosphorylation, and sulfation). In AD patient-derived astrocytes, this correlates with higher secretion of alarmin HMGB1, which can be blocked by CK2 inhibition. AD patient-derived neurons also have overactivated CK2 signaling and mitochondrial dysfunction. CK2 inhibition reduces tau phosphorylation (at PHF-1, or Ser396/404; FIG. 14), amyloid-β levels (Bettegazzi, et al., Cell Death Dis 12, 769 (2021)), and mutant huntingtin aggregates (Yu et al., Acta Neuropathologica Commun 10, 83 (2022); Gomez-Pastor et al., Nature Communications 8, 14405 (2017)). CK2 thus may play a multi-pathogenic role in certain neuroinflammatory diseases, such as AD, by blocking mitophagy and autophagy (FIG. 15), sustaining chronic inflammation, and leading to toxic protein aggregation.

Some embodiments of the disclosed compounds are inhibitors of CK2 enzyme activity. Inhibiting, or reducing, CK2 kinase activity reduces or blocks inflammatory signaling and or improves mitochondrial phenotypes, for example a reduction of inflammatory signaling and/or CK2 kinase activity of at least 50%, at least 60%, at least 70%, at least 80%, at least 90%, at least 95%, at least 98%, at least 99%, or even 100%, and/or an increase in mitochondrial phenotypes of at least 25%, at least 50%, at least 60%, at least 70%, at least 80%, at least 90%, at least 95%, at least 100%, at least 200%, at least 300%, at least 400%, or at least 500%, for example as compared to such activity in the absence of a compound according to formulas I-IV. Thus, in some examples, reducing CK2 kinase activity does not require 100% reduction in CK2 kinase activity. In any of the foregoing or following embodiments, the compound may be a targeted degrader where R is -L-Q, and inhibiting, or reducing, CK2 activity further comprises degrading the CK2 enzyme. Exemplary methods of measuring CK2 kinase activity are provided herein.

Embodiments of a method of reducing CK2 activity include contacting a cell that expresses CK2 enzyme with an effective amount of one or more compounds as disclosed herein, thereby reducing activity of the CK2 enzyme. Contacting may be performed in vitro, in vivo, or ex vivo. In some embodiments, the cell is an astrocyte, a microglia, a neuron, a white blood cell, an adipocyte, a myocyte, or an epithelial cell. The white blood cell may be a granulocyte (neutrophil, eosinophil, or basophil), a phagocyte (dendritic cell, monocyte, macrophage), a lymphocyte (T cell, B cell, natural killer (NK) cell)).

In any of the foregoing or following embodiments, reducing activity of the CK2 enzyme may reduce phosphorylation of one or more biomarkers, increase mitophagy, decrease mitochondrial fission, increase mitochondrial function, or any combination thereof, for example increase or decrease by at least 50%, at least 60%, at least 70%, at least 80%, at least 90%, at least 95%, at least 98%, at least 99%, at least 100%, at least 200%, at least 300%, at least 400%, or at least 500%, for example as compared to such activity in the absence of a compound according to formulas I-IV. In some embodiments, the one or more biomarkers are AKT1 (pS129), HMGB1, S100A9, SORCS1, IFI16, ILF2, IFNL1, ARFGAPI, RL6IP4, DTD1, SQSTM1, FERMT2, HDLBP, MAP4K4, NAV1, PNPLA6, SMC3, TMX2, IMMT, NF-κB, IκBα, FUNDC1, CK2 (pY255), or any combination thereof.

In any of the foregoing or following embodiments, contacting the cell with the compound may include administering a therapeutically effective amount of the compound, or a therapeutically effective amount of a pharmaceutical composition comprising the compound, to a subject. The subject may be an animal, such as a mammal. In some examples, the subject is a human. In some embodiments, the subject has a disease or condition characterized at least in part by dysregulated CK2 activity. The subject may be identified as having such a disease or condition by any suitable means as understood by a person skilled in the art, such as a physician or diagnostician. Suitable means for identifying the subject as having such a disease or condition may include laboratory tests, imaging, physical evaluation, and the like.

In some embodiments, administering the therapeutically effective amount of the compound or the therapeutically effective amount of the pharmaceutical composition comprising the compound to the subject ameliorates at least one sign or symptom of the disease or condition. “Ameliorate” means that at least one sign or symptom is reduced. In certain embodiments, the sign or symptom may be eliminated. Thus, administration of the compound may reduce severity of the disease or condition, slow progression of the disease or condition, or treat the disease or condition. In some embodiments, the compound may be administered on a prophylactic basis to prevent a disease or condition characterized at least in part by dysregulated CK2 activity.

In some embodiments, the disease or condition is characterized at least in part by inflammation, and administering the therapeutically effective amount of the compound or the therapeutically effective amount of the pharmaceutical composition comprising the compound to the subject may reduce the inflammation. In certain embodiments, the inflammation is neuroinflammation. In some examples inflammation or neuroinflammation is reduced by at least 50%, at least 60%, at least 70%, at least 80%, at least 90%, at least 95%, at least 98%, at least 99%, or even 100%, for example as compared to such activity in the absence of a compound according to formulas I-IV.

In any of the foregoing or following embodiments, the disease or condition may be cancer (e.g., cancers with mutated CSNK2A1 genotype or upregulated CK2 levels, such as cancers of the breast, lung, colon, and prostate), cardiac hypertrophy, cystic fibrosis, a neurodegenerative disease, bipolar disorder, depression, a viral infection, obesity, diabetes mellitus, atherosclerosis, epilepsy, or any combination thereof. Exemplary neurodegenerative diseases include, but are not limited to, Parkinson's disease, Huntington's disease, Alzheimer's disease, multiple sclerosis, and amyotrophic lateral sclerosis. Exemplary viral infections include, but are not limited to, coronavirus infections, such as SARS-CoV-2 infections. Advantageously, some embodiments of the disclosed compounds may be multi-targeting compounds, e.g., reducing neuroinflammation, mitochondrial defects, and/or toxic protein aggregation in central nervous system diseases.

In some examples, the disease or condition is a viral infection, such as a positive-strand RNA viral infection or negative-strand RNA viral infection. Exemplary positive-strand RNA viral infection includes, but are not limited to, infection with one or more of: Picornaviruses (such as Aphthoviridae [for example foot-and-mouth-disease virus (FMDV)]), Cardioviridae; Enteroviridae (such as Coxsackie viruses, Echoviruses, Enteroviruses, and Polioviruses); Rhinoviridae (Rhinoviruses)); Hepataviridae (Hepatitis A viruses); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); Calciviridae (which includes Norovirus and Sapovirus); and Coronaviruses (examples of which include SARS coronaviruses, such as the Urbani strain and SARS-CoV-2). Exemplary negative-strand RNA viral infections includes, but are not limited to infection with one or more of: Orthomyxyoviruses (such as the influenza virus), Rhabdoviruses (such as Rabies virus), and Paramyxoviruses (examples of which include measles virus, respiratory syncytial virus, and parainfluenza viruses). In some examples, the disease or condition is a DNA viral infection, such as: Herpesviruses (such as Varicella-zoster virus, for example the Oka strain; cytomegalovirus; and Herpes simplex virus (HSV) types 1 and 2), Adenoviruses (such as Adenovirus type 1 and Adenovirus type 41), Poxviruses (such as Vaccinia virus), and Parvoviruses (such as Parvovirus B19). In some examples, the disease or condition is a Retrovirus infection, such as human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); and avian sarcoma virus.

In some examples, the disease or condition is a cancer, such as a solid tumors such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the cancer is an adenocarcinoma, such as prostate adenocarcinoma. In some examples, the disease or condition is a liquid tumor, such as a lymphatic, white blood cell, or other type of leukemia. In a specific example, the cancer is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas).

The compound or pharmaceutical composition may be administered to the subject through any suitable route, and may be local or systemic. In some embodiments, the compound or pharmaceutical composition is administered to the subject by the oral route or in a single bolus delivery, via continuous delivery (for example, continuous transdermal, mucosal or intravenous delivery) over an extended time period, or in a repeated administration protocol (for example, by an hourly, daily or weekly, repeated administration protocol). In some embodiments, the compound or pharmaceutical composition is administered to the subject by injection. The therapeutically effective dosages of the agents can be provided as repeated doses within a prolonged prophylaxis or treatment regimen that will yield clinically significant results to alleviate one or more symptoms or detectable conditions associated with a targeted condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, avian, porcine, feline, non-human primate, and other accepted animal model subjects known in the art. Alternatively, effective dosages can be determined using in vitro models. Using such models, only ordinary calculations and adjustments are required to determine an appropriate concentration and dose to administer a therapeutically effective amount of the compound (for example, amounts that are effective to elicit a desired immune response or alleviate one or more symptoms of a targeted disease). In alternative embodiments, an effective amount or effective dose of the agents may simply inhibit or enhance one or more selected biological activities correlated with a disease or condition, as set forth herein, for either therapeutic or diagnostic purposes.

The actual dosages of the agents may vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the agent for eliciting the desired activity or biological response in the subject. Dosage regimens can be adjusted to provide an optimum prophylactic or therapeutic response. A therapeutically effective amount is also one in which any toxic or detrimental side effects of the agent are outweighed in clinical terms by therapeutically beneficial effects. A non-limiting range for a therapeutically effective amount of a compound according to any one of formulas I-IV within the methods and formulations of the disclosure is 0.001 mg/kg body weight to 100 mg/kg body weight, such as 0.01 mg/kg body weight to 20 mg/kg body weight, 0.01 mg/kg body weight to 10 mg/kg body weight 0.05 mg/kg to 5 mg/kg body weight, or 0.1 mg/kg to 2 mg/kg body weight. Dosage can be varied by the attending clinician to maintain a desired concentration at a target site (for example, systemic circulation). Higher or lower concentrations can be selected based on the mode of delivery, for example, trans-epidermal or oral delivery versus intravenous or subcutaneous delivery. Dosage can also be adjusted based on the release rate of the administered formulation, for example, of sustained release oral versus injected particulate or transdermal delivery formulations, and so forth.

In any of the foregoing or following embodiments, the therapeutically effective amount may be administered at intervals for a period of time effective to provide a therapeutic effect, e.g., amelioration of at least one sign or symptom of a disease or condition characterized at least in part by dysregulated CK2 activity. In some embodiments, the intervals are once daily. In other embodiments, the therapeutically effective amount may be divided into two or more doses administered at intervals in a 24-hour period. In some embodiments, the effective period of time is from one day to several months, such as from one day to 12 months, three days to six months, seven days to three months, 7-30 days, or 7-14 days. In certain embodiments, where the disease or condition is chronic, the effective period of time may be even longer than 12 months, such as a period of years.

In some examples, a subject treated one or more compounds provided herein to reduce CK2 enzyme activity can receive additional treatment with other compounds, such as one or more antiviral compounds (such as molnupiravir or remdesivir), one or more anti-inflammatory agents (such as a steroid, such as a corticosteroid), one or more chemotherapeutic agents, and one or more biologics (such as a monoclonal antibody used to treat cancer, such as one specific for PD1, EGFR (e.g., cetuximab), VEGF (e.g., bevacizumab), CD25, CTLA4, or a tumor-specific antigen such as HER2 (e.g., trastuzumab), CD52 (e.g., alemtuzumab), CD20, or CD19).

### V. EXAMPLES

### Methods

Cell culture. Primary fetal human cerebellar astrocytes (HCA, ScienCell) were cultured in Astrocyte Medium (AM, ScienCell). hESC and hiPSC cell lines were maintained on Matrigel (Cultrex)-coated plates in TeSR medium (made in-house by the Salk Stem Cell Core), fed daily and passaged with dispase every 5-7 days (Gibco). All subjects provided written informed consent and all procedures were approved by local human subjects committees. Mature hES-derived astrocytes were cultured in DMEM/F12 Glutamax (Thermo Fisher Scientific) supplemented with N2 and B27 (Thermo Fisher Scientific) and 10% fetal bovine serum (FBS, Biowest). Mature iPSC-derived microglia were cultured in DMEM/F12 Glutamax serum-free media (see below) supplemented with TGF-b1, IL-34, and M-CSF. THP-1 NFKB-Lucia reporter cells (InvivoGen) were grown in RPMI 1640 with 10% FBS, Glutamax, 25 mM HEPES pH=7.5, and penicillin/streptomycin (P/S), and differentiated into monocytes with 10 ng/mL PMA for 3 days. All cell lines were maintained in a humidified incubator (5% CO₂) at 37° C. and were routinely tested for mycoplasma.

Compounds. Phorbol 12-myristate 13-acetate was sourced from Fisher Scientific (NC9325685). Natural products were sourced as follows: chrysin (Sigma Aldrich), TMF, dTMF, chrysoeriol, homoeryodictyol (Indofine Chemical). Apigenin was a kind gift from the Mars Corporation. Naringenin was a kind gift from James LeClair. dO-TMF was synthesized according to the following conditions: dO-TMF was synthesized from TMF according to the following conditions: In a procedure adapted from Xiao et al (European Journal of Medicinal Chemistry 63 (2013) 685-695), a flame-dried 25 mL teardrop flask was charged with a Teflon stir bar and aluminum chloride (80 mg, 0.6 mmol, 3 eq.). The vessel was sealed with a rubber septum, evacuated via a needle inlet, and backfilled with argon. The vessel was charged with 1.1 mL dry tetrahydrofuran, and the resultant suspension was cooled with stirring in an ice-water bath. Lithium aluminum hydride solution (2.5 M in tetrahydrofuran, 0.12 mL, 1.5 eq.) was then added dropwise. Ten minutes later, a solution of trimethoxyflavone (TMF, 63 mg, 0.2 mmol, 1 eq) in dry tetrahydrofuran (4 ml) was added dropwise over five minutes. The resultant yellow mixture was stirred in an ice-water bath for another 125 minutes. It was then diluted with wet ethyl acetate followed by dropwise addition of deionized water, poured into a separatory funnel, and the layers were separated. The aqueous layer was extracted twice more with ethyl acetate. The combined organic fractions were washed with water and brine, dried over sodium sulfate, filtered, and concentrated at reduced pressure. Flash-column chromatography (6 by 4 cm SiO₂, 10% ethyl acetate/hexanes (250 mL)) of the crude residue generated 14 20 mL fractions. Fractions 6-12 were combined and concentrated to give dO-TMF as a white solid (38 mg, 0.128 mmol, 64%). 1H NMR (500 MHz, CDCl3, S): 7.59 (d, J=9 Hz, 2H); 6.90 (d, J=9 Hz, 2H); 6.21 (d, J=1 Hz, tH); 6.15 (d, J=1 Hz, 1H); 5.39 (t, J=4 Hz, 1H); 3.83 (s, 3H); 3.80 (s, 3H); 3.79 (s, 3H); 3.34 (d, J=4 Hz, 2H). Compounds were stored at −20° C. as 20 mM stock solution aliquots in DMSO.

General Synthesis Procedures. Flavones and quinolinones were synthesized according to the general procedures previously described in WO 2021/236578 A1.

Quinazolinones were synthesized according to the following general procedure.

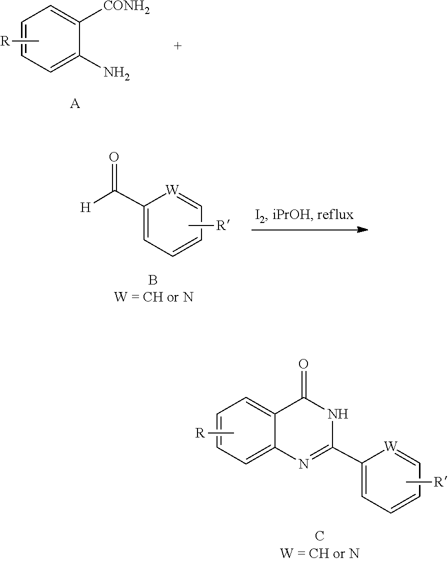

A mixture of the appropriate 2-aminobenzamide A (1 eq) and benzaldehyde or 2-pyridyl benzaldehyde B (1 eq) and iodine (5-10 eq) in isopropyl alcohol (0.5-1 M overall) was heated at reflux with vigorous stirring for two to four days. The reaction mixture was then cooled to room temperature and crashed out with saturated aqueous sodium thiosulfate. Isolation and drying of the precipitate by suction filtration furnished the requisite quinazolinone C in good yield and purity >90% as judged by proton NMR.

Aurones were synthesized according to the following general procedure.

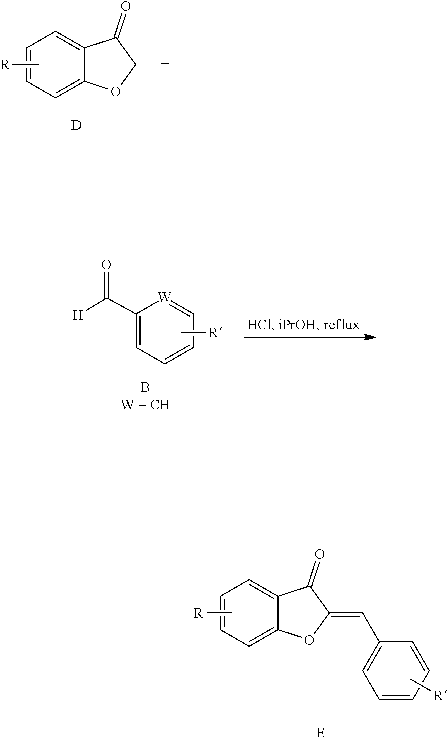

A mixture of the appropriate benzofuranone D (1 eq) and benzaldehyde B (W═CH, 1 eq) in isopropyl alcohol (0.2-0.6 M overall) was treated with one drop of 12 M HCl. The resultant mixture was heated at reflux with vigorous stirring for three to four hours. The reaction was then cooled to room temperature and crashed out with water. Isolation and drying of the precipitate by suction filtration furnished the requisite aurone E in good yield and purity >90% as judged by proton NMR.

2-Pyridyl aurones were synthesized according to the following general procedure.

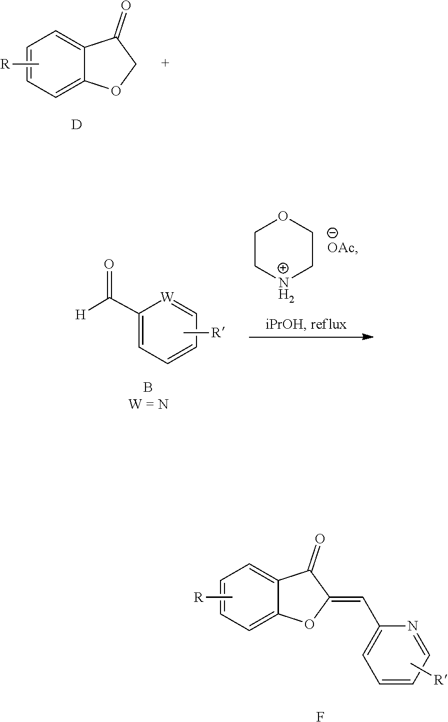

A mixture of the appropriate benzofuranone D (1 eq), 2-pyridylbenzaldehyde B (W═N, 1 eq), and morpholinium acetate (0.2 eq) in isopropyl alcohol (0.2 M overall) was heated at reflux with vigorous stirring overnight. The reaction was then cooled to room temperature and crashed out with water. Isolation and drying of the precipitate by suction filtration furnished the requisite 2-pyridyl aurone F in good yield and purity >90% as judged by proton NMR.

Differentiation of astrocytes from hES and hiPSC. H1 human embryonic stem cells (hESC, WiCell Research Institute) or AD and control iPSCs (UCI ADRC iPS Cell Bank) were cultured on Matrigel-coated plates in mTeSR medium and differentiated into glial progenitor cells (GPC) and mature astrocytes as previously described (Santos et al., Stem Cell Reports 2017, 8:1757-1769). First, embryoid bodies were prepared by mechanical dissociation of H1 cultures with 1 mg/mL collagenase IV (Gibco), plated onto ultra-low attachment plates (Corning) in TeSR medium with 10 M Y-27632 (ROCK inhibitor, StemCell Tech), and incubated overnight with rocking. For differentiation of GPCs from embryoid bodies, media was changed to AM supplemented with 500 ng/mL Noggin (R&D Systems) and 10 ng/mL PDGFAA (Peprotech) for 2 weeks and then Noggin was withdrawn for another week. The embryoid bodies were dissociated with papain (Papain dissociation system, Worthington) and the GPCs were cultured and expanded in 10 mg/mL poly-L-ornithine (Sigma)/1 mg/mL laminin (Invitrogen)-coated plates in AM supplemented with 20 ng/mL fibroblast growth factor 2 (FGF-2, Joint Protein Central) and 20 ng/mL epidermal growth factor (EGF, Humanzyme). Astrocytes were differentiated from low-confluent GPC cultures in DMEM/F12 Glutamax supplemented with N2 and B27 and 10% FBS. After 2 weeks of differentiation, the cells were transferred to non-coated plates for another 2 weeks of maturation. immunofluorescence staining was performed as described below to assess astrocytic marker expression with antibodies for Glast, S100B, vimentin.

Differentiation of microglia-like cells from iPSCs. H1 hESC and EC11 or Clue4-7 (Schafer et al., Nature Neuroscience 2019, 1-20) iPSC were cultured on Matrigel-coated plates in mTeSR medium and differentiated into mature microglia-like cells (iMGL) via an induced hematopoietic progenitor cell (iHPC) intermediate, as previously described (Abud et al., Neuron 2017, 94:278-293.e9). First, confluent stem cell cultures were dissociated into single-cell suspensions with TrypLE (Gibco) and plated onto untreated tissue-culture plates in TeSR with 10 μM Y-27632. The next day, media was changed to iHPC basal medium supplemented with FGF2 50 ng/ml, BMP4 50 ng/ml (Peprotech), Activin-A 12.5 ng/ml (Proteintech), 1 μM Y-27632, and 2 mM LiCl (Sigma), and placed in a hypoxic incubator (5% 02, 5% CO₂) for 4 days. iHPC basal medium was composed of 50% DMEM/F12, 50% IMDM, 2% ITS-X Insulin-Transferrin-Selenium-Ethanolamine (Thermo-Fisher Scientific), L-ascorbic acid 2-phosphate magnesium (64 μg/ml; Sigma), monothioglycerol (400 μM), PVA (10 μg/ml; Sigma), Glutamax (1×), chemically-defined lipid concentrate (1×), non-essential amino acids (NEAA; 1×), and 1% Penicillin/Streptomycin. Next, cells were cultured in normoxic conditions for another 6-16 days with fresh media supplemented with FGF2 50 ng/ml, VEGF 50 ng/mL (Proteintech), TPO 50 ng/ml (Proteintech), SCF 10 ng/ml (Proteintech), IL-6 50 ng/ml (Proteintech), and IL-3 10 ng/mL (Proteintech) added every 2 days. The resulting iHPCs were collected and sorted for CD43+ staining by FACS. Next, the pure iHPCs were differentiated into microglia-like cells by a 25-day maturation in iMGL medium (50% DMEM/F12, 50% IMDM, 2% ITS-G Insulin-Transferrin-Selenium (Thermo-Fisher Scientific), 2% B27, 0.5% N2, 200 μM monothioglycerol, 5 μg/mL insulin (Sigma), Glutamax (1×), NEAA (1×), 1% Penicillin/Streptomycin (ScienCell) supplemented with cytokines. For the first 22 days, cytokines M-CSF 25 ng/ml, IL-34 100 ng/ml, and TGFb-1 50 ng/ml (Proteintech) were added. Following a 3 day maturation with the addition of CD200 100 ng/ml and CX3CL1 100 ng/ml, the iMGLs were FACS-sorted using a 6-antibody panel. CD45+, CD11b+, CD64+, CD14+, CX3CR1+ and HLA-DR+ cells were plated onto 96-, 24-or 6-well Primaria plates (Corning) for downstream bead phagocytosis and activation assays.

Flow cytometry. For each well of HCA in a 6-well plate, 2 mL of fresh media with GolgiPLUG (BD #555029, 1:1000) and GolgiSTOP (BD #554724, 1:1000) with 20 μM compound or vehicle (DMSO) and IL1b 10 ng/mL or DPBS was added. The cells were incubated for 5 h at 37° C. Cells were collected with TrypLE and plated into a 96-well V-bottom plate for staining. The plate was centrifuged for 5 min, 1300 rpm, 4° C. to pellet cells, which were subsequently resuspended in 100 μL DPBS with 1 μL Zombie Violet (Biolegend #4231 13) and 5 μL Human Trustain FC block (Biolegend #422302) per well for 15 min at room temperature in the dark. The plate was centrifuged, supernatant was aspirated, and the cells were washed with 100 μL/well DPBS. Cells were fixed with 100 μL/well of Cytofix/Cytoperm (BD) for 20 min at 4° C. The plate was centrifuged, supernatant was aspirated, and cells were resuspended in 100 μL Permwash (BD) and incubated for 15 min at 4° C. The plate was centrifuged, supernatant was aspirated, and cells were incubated with 100 μL/well of Ab mix or isotype mix in Permwash (see below) for 20 min at 4° C., followed by centrifugation and 2× PermWash washes. The cells were resuspended in 100 μL DPBS and transferred to FACS tubes containing 200 ul of PBS (total volume per tube 300 μL) for analysis on FACS Analyzers (LSRII or Fortessa).

| Antigen | Fluorochrome | Vol. | Concentration | isotype | clone | catalog # | Company |
|---|---|---|---|---|---|---|---|
| IL-8 | PercP | 5 μL | 100 μg/mL | Mouse IgG2b | BH0814 | 514606 | Biolegend |
| TNF-a | Pe-Cy7 | 5 μL | 200 μg/mL | Mouse IgG1, k | MAb11 | 502930 | Biolegend |
| IL-6 | APC | 5 μL | 50 μg/mL | Rat IgG1, k | MQ2-13A5 | 501112 | Biolegend |
| MCP-1 | PE | 3 μL | 200 μg/mL | Armenian | 2H5 | 505904 | Biolegend |
| (CCL2) | | | | Hamster IgG | | | |

FACS sorting. For FACS sorting of iHPC and iMGL, cells were collected and filtered through a 70 μM mesh cell strainer attached to a 50 mL conical tube to remove large clumps and centrifuge at 1000 rpm for 3 min. The pellet was washed with 3 mL of cold sterile filtered FACS buffer (1×DPBS, 2% BSA, and 0.05 mM EDTA pH 8.0) and transferred to a 15 mL conical tube centrifuge. They were resuspended in 100-300 μL of FACS buffer, 5 μL/100 μL of Trustain was added, and the cells were incubated at RT for 5 min. 5 μL of cells were removed from the sample and transferred to a new conical tube, brought to 100 ul of FACS buffer and 5 μL of corresponding isotype control antibody (see below) was added along with 1 μL of Zombie Violet live/dead stain. For each sample, 5 μL of each antibody (see below) and 1 μL-3 μL of Zombie Violet live/dead stain was added. Each sample and the isotype control was incubated on ice in the dark for 20 min. After staining, the samples were washed once with 1 mL of cold FACS buffer, centrifuged and resuspended in 100-300 μL FACS buffer. The sample and the isotype control were transferred into separate FACS tubes with a 40 μM filter attached. For iHPC, all live and CD43+ cells were sorted into cold basal iHPC differentiation medium including Pen/Strep. For iMGL, all live and CD45+, CD11b+, CD64+, CD14+, CX3CR1+ and HLA-DR+ cells were sorted into cold basal iMGL differentiation medium.

| | | | | Concentration |
|---|---|---|---|---|
| Color | Antibody | Catalog # | Volume (μL) | (μg/mL) |
| APC | CD64 | 305014 | 5 | 200 |
| PerCP-Cy5.5 | CX3CR1 | 341614 | 5 | 400 |
| 488/FITC | CD14 | 325610 | 5 | 400 |
| PE | CD11b | 301306 | 5 | 200 |
| PE-Cy7 | HLA-DR | 307616 | 5 | 200 |
| APC-Cy7 | CD45 | 304014 | 5 | 100 |
| FITC | CD43 | 315204 | 5 | 400 |
| FITC | Isotype Ct | 400107 | 5 | 500 |
| APC | Isotype Ct | 400122 | 5 | 200 |
| PerCP-Cy5.5 | Isotype Ct | 401623 | 5 | 200 |
| PE | Isotype Ct | 400112 | 5 | 200 |
| PE-Cy7 | Isotype Ct | 400231 | 5 | 200 |
| APC-Cy7 | Isotype Ct | 400127 | 5 | 200 |

IL-6 secretion assay. HCAs were plated at 20,000 cells per well in white clear bottom 96-well plates. Two to three days later, media was removed and replaced with media containing 10 ng/mL IL1B and compound dilution series. Five hours later, plates were processed using the Lumit IL-6 Assay Kit (Promega W6030) according to the manufacturer's instructions.

NF-κB-Luciferase reporter assay. THP-1 NF-κB-Lucia cells were plated at 10,000 cells per well with 10 ng/mL PMA. Three days later, media was removed and replaced with media containing LPS (1 μg/mL) and dilution series of compounds in flat bottom clear 96-well plates for 24 hours. A day later, 20 μL of media was transferred to a 96-well white plate and 50 μL of QuantiLuc Gold reagent (InvivoGen rep-qlcg5) was added per well; plate was mixed in the plate reader and luminescence was read using a Promega GloMax Reader.

Cytokine release assay. Microglia were treated in 6-well plates with fresh iMGL media and either vehicle alone (DMSO) or 200 ng/mL lipopolysaccharide (LPS, Sigma) with 20 μM CHR or vehicle for 5 hours in duplicate. Subsequently, media cleared of cells and debris was collected, snap-frozen and stored at −80° C. until processing with the Human Cytokine Array kit (R&D Systems, Catalog #ARY005). Briefly, media was diluted in Array Buffer 4 and incubated with a cocktail of biotinylated detection antibodies for 1 h at room temperature with rocking. The resulting immunocomplexes were incubated with nitrocellulose membranes spotted with capture antibodies overnight at 4° C. (see below for full list of targets). The membranes were washed 4× with Wash Buffer, then incubated with IRDye 800CW Streptavidin for 30 minutes at room temperature (1:2000 in Array Buffer 5, LI-COR #926-32230). The membranes were washed 4× and imaged with an Odyssey CLx instrument. Intensity for each spot was quantified with ImageStudio. Technical duplicate values for each target were averaged, and differential cytokine release by microglia exposed to LPS+vehicle versus vehicle only was determined (n=2 biological replicates, two-tailed t-tests, P<0.05). Then, it was determined whether the release of those cytokines was significantly affected by CHR treatment (n=2 biological replicates, two-tailed t-tests, P<0.05).

| | | |
|---|---|---|
| C5a | IL-4 | IL-32 alpha |
| CD40 ligand | IL-5 | CXCL10/IP-10 |
| G-CSF | IL-6 | CXCL11/I-TAC |
| GM-CSF | IL-8 | CCL2/MCP-1 |
| CXCL1/GRO alpha | IL-10 | MIF |
| CCL1/I-309 | IL-12 p70 | CCL3/MIP-1 alpha |
| ICAM-1 | IL-13 | CCL4/MIP-1 beta |
| IFN-gamma | IL-16 | CCL5/RANTES |
| IL-1 alpha | IL-17 | CXCL12/SDF-1 |
| IL-1 beta | IL-17E | Serpin E1/PAI-1 |
| IL-1ra | IL-23 | TNF-alpha |
| IL-2 | IL-27 | TREM-1 |

Phagocytosis assay. Microglia were pre-treated in 12-well plates with fresh iMGL media with vehicle (DMSO), 20 μM CHR, or 20 μg/mL cytochalasin D for 1 h at 37° C. (2-3 biological replicates). Then, 0.5 μL of pHrodo E. Coli-FITC beads (Thermo Fisher, P35366) was added per well with gentle mixing and the cells were incubated for another 4 hours. pHrodo beads only fluoresce when they are internalized in cells. Cells were then collected by scraping, washed with FACS buffer (1×DPBS, 2% BSA, and 0.05 mM EDTA pH 8.0), and counter-stained with Zombie Violet to assess viability. Live cells were analyzed on the FACS Canto instrument for geometric mean fluorescence on the FITC channel. The background FITC fluorescence value (pHrodo beads only) was subtracted from each mean, and fold-change was computed across experimental groups.

Western blotting. For drug treatment assays, cells were treated with vehicle (DMSO) or 20 μM compound and stimulated with IL1B 10 ng/mL for 1 h, 6 h, or 24 h. Cells were dissociated with 1:1 Accutase:papain for 3 min at rt, pelleted for 5 min at 800 g, and washed twice with cold DPBS. Pellets were lysed with RIPA buffer (Sigma R⁰²⁷⁸) supplemented with Halt protease inhibitor cocktail (Thermo Fisher 78445) and clarified by centrifugation (16,000 g, 10 min, 4° C.). Protein concentration was determined by BCA assay (Thermo Fisher). Proteins were denatured in 1×LDS Buffer and 2.5% beta-mercaptoethanol (Sigma) for 10 min at 70° C., separated on Bolt 4-12% Bis-Tris Plus polyacrylamide gels (Thermo Fisher), transferred to PVDF membranes using the iBlot 2 Dry Blotting System, and blocked with 0.1% casein-PBS (Bio-Rad) for 1 h at room temperature. The blots were incubated with primary antibodies overnight at 4° C. in PBS containing 0.1% casein and 0.2% Tween-20. Antibodies used: rabbit anti-CK2α1 (Bethyl #, 1:5000), rabbit anti-CK2α2 (Bethyl #, 1:5000), mouse anti-GAPDH (Fitzgerald #10-1501, 1:20,000), mouse anti-NFKB (CST #6956S, 1:1000), pNFKB S529 (ThermoFisher #14-9864-82, 1:100), rabbit HMGB1 (Novus #NB1002322, 1:1000), mouse anti-IkBa (CST #4814S, 1:1000), mouse anti-IkBa pS32 (SC #8404, 1:500), HA-tag (Bethyl #A190-108A, 1:20000). The membranes were washed 5× with 0.1% PBS-Tween20 (PBST) and incubated with secondary antibodies (1:20,000) for 1 h at rt in PBS with 0.1% casein, 0.2% Tween20, and 0.1% SDS. Secondary antibodies: goat anti-mouse IgG IRDye 800CW #925-32210, goat anti-rabbit mouse IgG IRDye 800CW #925-32211, goat anti-mouse IgG IRDye 680RD #926-68070, or goat anti-mouse IgG IRDye 680RD #926-68071. The blots were then washed 4× with PBST and 1× with DPBS. The blots were imaged using the Odyssey CLx imaging system and blots were analyzed on the ImageStudio software (LI-COR).

Immunofluorescence. Primary human astrocytes (ScienCell) were plated on glass slides (EMD Millipore PEZGS0896) and treated with CHR, CX-4945/silmitasertib, or DMSO and stimulated with IL1B for 1 hour, 5 hours or 25 hours. After treatment, cells were fixed with 4% paraformaldehyde solution for 20 min at room temperature and washed 3× with DPBS for 10 min each. The cells were permeabilized using 5% horse serum and 0.1% Triton X in DPBS for 15 min at room temperature and blocked with 5% horse serum in PBS for 30 min at room temperature. Cells were incubated with primary antibodies in blocking buffer either overnight at 4° C. or for 2 hours at room temperature. After incubation, cells were washed 3× with PBS, blocked using 5% horse serum (Sigma H1270) in PBS for 30 min at room temperature, and incubated with Cy3 red-labeled or AF488 green-labeled secondary antibodies diluted in blocking buffer for 1 hour at room temperature. The cells were washed once with PBS for 10 min, then counter-stained with DAPI. The cells were washed 3× with PBS and mounted with glass coverslips and Immu-Mount solution. Images were obtained with a Zeiss confocal microscope (Zeiss Axio Observer Z1). The following primary antibodies were used: rabbit anti-NFkB (1:400; CST), mouse anti-phospho NFkB S529 (20 ug/ml; Thermo Fisher), rabbit anti-CK2A1 (1:500; Bethyl), mouse anti-IKb alpha (1:400; CST), mouse anti-phospho TKb alpha (1:50; Santa Cruz), rabbit anti-phospho CK2A1 (1:25; Thermo Fisher), rabbit anti-phospho Cdc37 (1:50; Novus), mouse anti-phospho NFkB S529 (1:25; R&D), rabbit anti-Cdc37 (1:100; CST), mouse anti-SQSTM (1:50; Santa Cruz), anti rabbit-phospho NFkB S536 (1:1600; CST), anti rabbit-Akt (pan) (1:400; CST), anti mouse-phospho Akt S473 (1:500; Sigma).

Activation assays and ddPCR. HCA or H1-derived astrocytes were treated with vehicle (DMSO) or 20 μM compound and stimulated with IL1B 10 ng/mL for 5 h. Cells were scraped into RNA-Bee and total RNA was purified using the Direct-zol RNA kit (Zymo Research). RNA concentration was measured using Nanodrop and RNA was reverse transcribed into cDNA using the High Capacity cDNA Reverse Transcriptase Kit (Applied Biosystems). Reaction mixes consisting of TaqMan FAM probe (IL6 probe Hs00174131_m1, IL8 probe Hs00174103_m1, CSNK2A1 probe Hs00751002_s1, CSNK2A2 probe Hs00176505_m1), control TaqMan ACTB-VIC probe (Thermo Fisher Scientific 4326315E), ddPCR Supermix for Probes (Bio-Rad 186-3024), and cDNA were formed into oil droplets using the QX200 Droplet Generator. After amplification (Bio-Rad C1000 Touch Thermal Cycler) according to manufacturer's instructions, the plate was read in the QX200 Droplet Reader.

Cloning and lentiviral transduction. pBOB-CAG-CK2α1-WT, pBOB-CAG-CK2α2-WT, pBOB-CAG-CK2a1-K68M, pBOB-CAG-CK2a1-K69M plasmids were generated by cloning HA-tagged CK2 inserts from parent plasmids (Litchfield lab, Addgene 27086, 27090, 27089, 27087) into the pBOB backbone (Addgene #12337). Plasmids were transformed into TOP10 competent cells (Thermo Fisher). H1-GPCs in 6-well plates were transduced with lentiviral particles (~10⁸ particles/mL, Salk Virus Core) by incubating for 3 days. Media was changed and cells were differentiated into mature astrocytes as described above after checking for HA-CK2 expression by Western blotting. Astrocytes were processed as described under “Activation assays, ddPCR”.

siRNA knockdown. HCAs were nucleofected with siRNAs against CSNK2A1 (Ambion s2888), CSNK2A2 (Ambion s7501), or scrambled control (Ambion 4390843) using the Amaxa Nucleofector II (Program T-019). After 48 hours, knockdown efficiency was assessed by ddPCR and cells were replated to assess inflammatory response to IL1b as described under “Activation assays, ddPCR”. Results shown in figures represent multiple electroporations.

CRISPR-Cas9 editing. THP-1-NFKB-Lucia cells were genome-edited using CRISPR-Cas9 ribonucleotide particles (RNP). 2 million cells were washed with DPBS and resuspended in 100 μL of Ingenio Electroporation Buffer (Mirus Bio 10766-842). For the CK2 knockouts, custom order TrueGuide synthetic gRNAs by Thermo-Fisher (A35533) were used, sgCK2A1 (SEQ ID NO 1: GTGAGGATAGCCAAGGTTCT) and sgCK2A2 (SEQ ID NO 2: ACGCCGAGGTGAACAGTCTG). For the NFKB knockin, two custom order TrueGuide synthetic gRNAs by Thermo-Fisher (A35533) were used, sgNFKB-G526 (SEQ ID NO 3: ATCTCCTGAAAGGAGGCCAT) and sgNFKB-stopCodon (SEQ ID NO 4: AGGGCAGGCGTCACCCCCTT). For each CRISPR-Cas9 editing experiment, 8 μg of Cas9 (TrueCut V2, Thermo-Fisher A36498) was mixed with 0.48 μL of each sgRNA (100 μM stock) and incubated 15 min at rt. The RNP complex was mixed with 0.48 μL electroporation enhancer (IDT 1075916) and added to cells in Ingenio Electroporation Buffer. For the knockin experiment, 9 μL HDR donor (HDR—NFKB-WT-NeoR-v2, 325 ng/μL) was also added at this step. HDR donor DNA was synthesized by Synbio Technologies. The cells were nucleofected using program V-001 on the Amaxa II Nucleofector, then added to 2 mL THP-1 media (plus HDR enhancer v2 at 1 μM (IDT 10007910, 1:690) for the knockin experiment). After 3 weeks recovery, cells were selected with 100 ug/mL G418 (Gibco 10131027) for 1 week. For CK2A1 and CK2A2 knockouts, clones were isolated using FACS sorting into 96-well plates and validated by Western blotting, immunoprecipitation-Western, and/or Sanger sequencing.

Thermal shift assay and TPP. Experiments were performed as previously described (Franken et al., Nature Protocols 2015, 10:1567-1593; Reinhard et al., Nature Methods 2015, 12:1129-1131). 45 million H1-derived astrocytes were activated with 10 ng/mL IL1b for 6 h, dissociated with 1:1 Accutase/papain (3 min rt), and centrifuged at 1,800 rpm for 2 min at 4 C. The pellet was washed twice with 10 mL cold DPBS, lysed in 2.25 mL DPBS and 0.4% v/v NP-40 by freeze-thawing 3×, and clarified by centrifugation (20,000 g, 30 min, 4° C.). Protein concentration was determined by BCA assay (Bio-Rad), and lysates were diluted to 2 mg/mL. First, compound or vehicle was added (10 μL DMSO, 10 μL of 20 mM TMF, or 10 μL of 20 mM CHR) to individual tubes, then 0.99 mL lysate was added to each tube and vortexed briefly. Final concentrations: 1% vehicle, 200 M TMF, 200 μM CHR. Reactions were incubated at rt for 30 min, after which each treated extract was divided into 10 aliquots of 95 μL in PCR strip tubes and stored on ice. Each group of treated samples was heated for 3 min at predesignated temperatures (3×10 temperatures), then kept for 3 min at rt before ultracentrifugation (20 min at 4° C., 100,000 g) in 200 μL polycarbonate tubes (Beckman Coulter #343775). Designated temperatures were 37, 41.1, 43.6, 46.9, 50, 53.7, 56, 59.5, 63, and 67° C. A small aliquot (5 μL) of supernatant was saved for Western blotting, and the rest was snap frozen at −80 C and submitted for LC-MS/MS. For isothermal dose-response experiments, CHR was added to individual extracts at concentrations in a 3-fold dilution series ranging from 200 μM-0.1 μM, including one vehicle control, and the extracts were heated at 60° C. and processed as described above.

TPP analysis. MS data was analyzed using the TR workflow of the R package TPP (version 3.10.0) as previously described (Franken et al., Nature Protocols 2015, 10:1567-1593).

The package performs protein quantity normalization, fits melting curves, determines melting points, and identifies proteins that have a significant shift in thermal stability compared with controls. Data from two independent TPP experiments were used to quantify significant thermal shifts between CHR and vehicle and TMF and vehicle. The algorithm was run with minor changes to default parameters (filtering requirements changed to include peptides with spectral counts ≥2 and fold-change thresholds from 0-1.5). Briefly, data for each protein and condition underwent curve-fitting analysis and significance of thermal shift was calculated only for proteins with R²>0.8 and a plateau of <0.3 for the vehicle curve. Proteins that met all 4 of the following benchmarks for significance and had spectral counts ≥3 were considered “hits”: (a) P values for the two replicate experiments were <0.05 and <0.2, respectively. (b) The compound-vehicle melting point shifts in the two independent experiments had the same direction. (c) Each compound-vehicle ΔTₘ was greater than the ΔT between the two vehicle controls. (d) The minimum curve slope in each experiment was <−0.06.). Network analysis and visualization of each set of hits was performed with the GeneMania app within Cytoscape (v. 3.5.1).

Native kinase capture. Experiments were performed as previously described (Patricelli et al., Chem Biol 2011, 18:699-710). 45 million H1-derived astrocytes were activated with 10 ng/mL ILlb for 6 h, dissociated with 1:1 Accutase/papain (3 min rt), and centrifuged at 1,800 rpm for 2 min at 4 C. The pellet was washed twice with 10 mL cold DPBS, lysed in 2.25 mL DPBS by repeated freeze-thawing, sonicated (2×10 s pulses with a 30 s break, 4° C.) and clarified by centrifugation (20,000 g, 10 min, 4° C.). The supernatant was desalted through a column (732-2010, Biorad) and then eluted with cold kinase buffer (20 mM HEPES, pH 7.4, 150 mM NaCl, 0.5% Triton X-100, with Halt Protease and Phosphatase inhibitor cocktail). For each treatment, 475 μL of the lysate (2 mg/mL) was pre-incubated with 10 μL MnCl₂ (1 M) and 5 μL compound to the desired concentration at room temperature for 30 min. Uninhibited kinases were captured with 10 μL ActivX desthiobiotin-ATP probe (0.25 mM; 88311, Pierce) at room temperature for 10 min. Samples were mixed with 500 μL urea (8 M; 818710, Millipore) and 50 μL streptavidin agarose (20359, Thermo) for 60 min at room temperature on a nutator. Beads were washed twice with a 1:1 mixture of kinase buffer and 8 M urea, and collected by centrifugation (1,000 g, 1 min). Proteins were eluted from the beads with 100 μL 2×LDS sample buffer (NP0007, Life) at 95° C. for 10 min. Samples were analysed by standard immunoblotting. Experiment was performed twice.

Mass Spectrometry. Samples were precipitated by methanol/chloroform and redissolved in 8 M urea/100 mM TEAB, pH 8.5. Proteins were reduced with 5 mM tris(2-carboxyethyl)phosphine hydrochloride (TCEP, Sigma-Aldrich) and alkylated with 10 mM chloroacetamide (Sigma-Aldrich). Proteins were digested overnight at 37° C. in 2 M urea/100 mM TEAB, pH 8.5, with trypsin (Promega). The digested peptides were labeled with 10-plex TMT (Thermo product 90309), pooled samples were fractionated by basic reversed phase (Thermo 84868). The TMT labeled samples were analyzed on a Fusion Lumos mass spectrometer (Thermo). Samples were injected directly onto a 25 cm, 100 m ID column packed with BEH 1.7 μm C18 resin (Waters). Samples were separated at a flow rate of 300 nL/min on a nLC 1200 (Thermo). Buffer A and B were 0.1% formic acid in water and 90% acetonitrile, respectively. A gradient of 1-25% B over 180 min, an increase to 40% B over 30 min, an increase to 100% B over another 20 min and held at 90% B for a 10 min was used for a 240 min total run time. Peptides were eluted directly from the tip of the column and nanosprayed directly into the mass spectrometer by application of 2.8 kV voltage at the back of the column. The Lumos was operated in a data dependent mode. Full MS1 scans were collected in the Orbitrap at 120 k resolution. The cycle time was set to 3 s, and within this 3 s the most abundant ions per scan were selected for CID MS/MS in the ion trap. MS3 analysis with multinotch isolation (SPS3) was utilized for detection of TMT reporter ions at 60 k resolution (ref5). Monoisotopic precursor selection was enabled and dynamic exclusion was used with exclusion duration of 10 s. Protein and peptide identification were done with Integrated Proteomics Pipeline—IP2 (Integrated Proteomics Applications). Tandem mass spectra were extracted from raw files using RawConverter (ref1) and searched with ProLuCID (ref2) against the Uniprot human database. The search space included all fully-tryptic and half-tryptic peptide candidates. Carbamidomethylation on cysteine and TMT on lysine and peptide N-term were considered as static modifications. Data were searched with 50 ppm precursor ion tolerance and 600 ppm fragment ion tolerance. Identified proteins were filtered to using DTASelect (ref3) and utilizing a target-decoy database search strategy to control the false discovery rate to 1% at the protein level (ref 4). Quantitative analysis of TMT was done with Census (ref 6) filtering reporter ions with 20 ppm mass tolerance and 0.6 isobaric purity filter.
(1) Lin He, Jolene Diedrich, Yen-Yin Chu, and John R. Yates, I I I. Extracting Accurate Precursor Information for Tandem Mass Spectra by RawConverter. Analytical Chemistry 2015 87 (22), 11361-11367, DOI:10.1021/acs.analchem.5b02721
(2) Xu T, Park S K, Venable J D, Wohlschlegel J A, Diedrich J K, Cociorva D, Lu B, Liao L, Hewel J, Han X, Wong C C, Fonslow B, Delahunty C, Gao Y, Shah H, Yates J R 3rd. ProLuCID: An improved SEQUEST-like algorithm with enhanced sensitivity and specificity. J Proteomics. 2015 Nov. 3; 129:16-24. doi: 10.1016/j.jprot.2015.07.001.
(3) Tabb, D. L. et al. (2002) DTASelect and Contrast: tools for assembling and comparing protein identifications from shotgun proteomics. J. Proteome Res., 1(1), 21-26.
(4) Peng J, Elias J E, Thoreen C C, Licklider L J, Gygi S P. J Proteome Res. 2003 January-February; 2(1):43-50. Evaluation of multidimensional chromatography coupled with tandem mass spectrometry (LC/LC-MS/MS) for large-scale protein analysis: the yeast proteome. Graeme C. McAlister, David P. Nusinow, Mark P. Jedrychowski, Martin Wdhr, Edward L. Huttlin, Brian K. Erickson, Ramin Rad, Wilhelm Haas, and Steven P. Gygi. MultiNotch MS3 Enables Accurate, Sensitive, and Multiplexed Detection of Differential Expression across Cancer Cell Line Proteomes. Anal Chem. 2014 Jul. 15; 86(14): 7150-7158. PMCID: PMC4215866 PMID: 24927332
(5) Park S K, Aslanian A, McClatchy D B, Han X, Shah H, Singh M, Rauniyar N, Moresco J J, Pinto A F, Diedrich J K, Delahunty C, Yates J R 3rd. Census 2: isobaric labeling data analysis. Bioinformatics. 2014 Aug. 1; 30(15):2208-9. doi: 10.1093/bioinformatics/btul51.
   Immunoprecipitation. HCAs were treated for 2 hours with vehicle or CHR 20 μM and ILIb 10 ng/mL or vehicle only. Cells were dissociated with Accutase and centrifuged at 1,800 rpm for 5 min at 4 C. The pellet was washed twice with cold DPBS, lysed in IP Buffer (20 mM HEPES 7.4 pH, 150 mM NaCl, 1% Triton-X, 1 mM EDTA, 1 mM EGTA plus Halt Protease and Phosphase Inhibitor Cocktail) with sonication (6×10 s pulses with 10 s breaks, 4° C.) and clarified by centrifugation (20,000 g, 10 min, 4° C.). Lysates were precleared with Protein A/G Magnetic Beads (Pierce) for 20 min at rt, then incubated with NFKB primary antibody (Novus NB100-97831, Rabbit polyclonal) overnight with rocking at 4° C. After three washes in cold DPBS, proteins were eluted in 4×LDS/BME at room temperature for 10 minutes, then denatured at 70° C. for 10 minutes, separated on NuPAGE gels at 1×LDS, and probed with NFKB primary antibody (CST D14E12) and anti-rabbit Light Chain-specific IgG AlexaFluor 790 (Jackson Immuno NC0493011) secondary antibody for detection.
   RNA-seq in primary and iPSC-derived astrocytes. RNA-sequencing analysis was performed on 5-week-old HCAs, either non-stimulated and stimulated with 10 ng/mL IL-1β, further split into two groups, without or with apigenin (20 μM). 5 hours later, the cells were harvested in RNABee solution (Tel test, Inc) and total RNA was extracted using the DNAase-Free RNA Kit (Zymo Research) according to the manufacturer's instructions. iPSC-derived astrocytes were differentiated for four weeks from GPCs in SATO media (50% DMEM Glutamax, 50% Neurobasal, 100×SATO supplement [Neurobasal media, transferrin 100 ug/ml, BSA 100 [μg/mL, putrescine 16 g/mL, progesterone 60 ng/mL, sodium selenite 40 ng/mL], 1 mM Glutamine (200×), 1 mM Sodium Pyruvate (100×), 20 ng/mL CNTF, 20 ng/mL BMP4, 5 μg/mL insulin, 20 ng/mL EGF, 20 ng/mL FGF2) in 6-well plates (triplicate wells, n=3 CT, n=3 AD lines). Media was changed 2 days before harvesting RNA to the following: 50% DMEM (no phenol red), 50% Neurobasal (no phenol red), 20 ng/mL carrier-free CNTF, 20 ng/mL carrier-free BMP4, 1 mM sodium pyruvate, 1 mM Glutamax, and 100 g/mL Penn/Strep. RNA quality was assayed using Agilent Technologies 2200 TapeStation and only samples with high RNA quality (RIN>8) were used for library preparation. Stranded mRNA-Seq libraries were prepared using the Illumina TruSeq Stranded mRNA Library Prep Kit according to the manufacturer's instructions.
   RNA-seq analysis of primary astrocytes. Sequenced reads were quality-tested using FASTQC and aligned to the hg19 human genome using the STAR aligner version 2.4.0k. Mapping was carried out using default parameters (up to 10 mismatches per read, and up to 9 multi-mapping locations per read). The genome index was constructed using the gene annotation supplied with the hg19 Illumina iGenomes collection and overhang value of 100. Raw gene expression was quantified across all gene exons (RNA-Seq) using the top-expressed isoform as proxy for gene expression. Differential expression was performed using DESeq2 package version 1.20.0. Differentially expressed genes were defined as having a false discovery rate (FDR)<0.05 and a log₂ fold change >1. Enrichment analysis was performed using WebGestaltR package version 0.1.1. Hierarchical clustering was performed using the R language (v.3.3.2) with Ward's hierarchical agglomerative clustering method and 1-correlation as a distance metric.
[1] Andrews S. (2010). FastQC: a quality control tool for high throughput sequence data. Available online at: http://www.bioinformatics.babraham.ac.uk/projects/fastqc
[2] The Genome Sequencing Consortium. Initial sequencing and analysis of the human genome. Nature. 2001 Feb. 15; 409(6822):860-921
[3] Dobin A, Davis C A, Schlesinger F, Drenkow J, Zaleski C, Jha S, et al. STAR: ultrafast universal RNA-seq aligner. Bioinformatics. 2013; 29(1):15-21. doi: 10.1093/bioinformatics/bts635. pmid:23104886
[4] iGenomes online. Illumina. 2015. http://support.illumina.com/sequencing/sequencing_software/igenome.html
[5] Love, M. I., Huber, W., & Anders, S. (2014). Moderated estimation of fold change and dispersion for RNA-seq data with DESeq2. Genome biology, 15(12), 550.
[6] Wang, J., Duncan, D., Shi, Z., & Zhang, B. (2013). WEB-based gene set analysis toolkit (WebGestalt): update 2013. Nucleic acids research, 41(W1), W77-W83.
   RNA-seq analysis of iPSC-derived astrocytes. Sequenced reads were quality-tested using FASTQC and reads were trimmed using SolexaQA v3.1.7.1 and adapters were removed using cutadapt v2.10. Reads were aligned to the hg38 human genome and quantified using Salmon v1.2.1 using default parameters in mapping-based mode. The pre-computed genome index with partial decoy was downloaded from https://refgenomes.databio.org/v2/asset/hg38/salmon_partial_sa_index/archive?tag=default. Quantified gene expression data was loaded into R v4.0.0 and annotated to Ensembl identifiers using package tximeta and converted gene symbols with package org.Hs.eg.db. Differential expression was performed using the glm function of edgeR package version 3.11. Differentially expressed genes were defined as having a false discovery rate (FDR)<0.05 and a log 2 fold change >0.69. Enrichment analysis was performed using DAVID v6.8 and ClueGO v2.5.7 (Cytoscape 3.5.1 plugin).
[1] Cox, M. P., D. A. Peterson, and P. J. Biggs. 2010. SolexaQA: At-a-glance quality assessment of Illumina second-generation sequencing data. BMC Bioinformatics 11:485.
[2] Martin, Marcel. Cutadapt removes adapter sequences from high-throughput sequencing reads. EMBnet.journal, [S.1.], v. 17, n. 1, p. pp. 10-12, May 2011. doi:https://doi.org/10.14806/ej.17.1.200.
[3] Patro, R., Duggal, G., Love, M. I., Irizarry, R. A., & Kingsford, C. (2017). Salmon provides fast and bias-aware quantification of transcript expression. Nature Methods.
[4] Robinson M D, McCarthy D J, Smyth G K (2010). “edgeR: a Bioconductor package for differential expression analysis of digital gene expression data.” Bioinformatics, 26(1), 139-140. doi: 10.1093/bioinformatics/btp616.
[5] Shannon P, Markiel A, Ozier O, Baliga N S, Wang J T, Ramage D, Amin N, Schwikowski B, Ideker T. Cytoscape: a software environment for integrated models of biomolecular interaction networks. Genome Research 2003 November; 13(11):2498-504.
   Cmap analysis. L1000 data was accessed using clue.io cloud-based software. The list of differentially-expressed genes from API/IL1b-treated astrocytes compared to IL1b-treated astrocytes was uploaded to CLUE, then searched against overexpression and knockdown signatures present in L1000 data using the Morpheus module. The resulting signatures were rank-ordered and plotted in GraphPad Prism from least to most similar.

Analysis of Aging, Dementia, and TBI RNA-seq data and UPP Proteomics data. Normalized RNA-seq data (as z-scores) and sample metadata for CSNK2A1, CSNK2A2, CSNK2A3 and CSNK2B expression in the parietal cortex was downloaded from https://aging.brain-map.org/download/index. Data was filtered to retain only samples from patients who had never experienced TBI and who were classified as having “Dementia” (n=21) or “No Dementia” (n=27), and individual two-tailed t-tests were conducted to determine statistically significant differences; only CSNK2A2 exhibited differential expression between dementia and controls. For the UPP Proteomics study, normalized and batch-corrected log 2(abundance) data for CSNK2A1, CSNK2A2, CSNK2A3 and CSNK2B was downloaded from 10.7303/syn17009177. Data was filtered to retain only samples from patients with AD (n=44) or age-matched controls (n=48), and one-way ANOVA with Sidak's post-hoc test was conducted to determine statistically significant differences; only CSNK2A2 exhibited differential expression between AD and controls.

(Promega #NV1191) according to the manufacturer's instructions. ~1M HEK293T cells were transfected with 2.5 ug of NanoLuc-CSNK2A2 plasmid (Promega #PRN2500) using Lipofectamine 2000, and incubated for 24 h. Cells were dissociated with TrypLE 3 min at rt, inactivated by adding media, and spun down at 300 g for 3 min. 20,000 cells were seeded per well in DMEM+10% FBS media into white, non-binding surface, polypropylene 96-well plates and left to attach overnight. Test compounds were prepared by diluting 1,000× stocks to 1× final concentration in Assay Medium and performing 2-fold serial dilutions. NanoBRET tracer was added to 1× final concentration to all compound concentrations. Media was aspirated and a 100 μl of 1× serially diluted test compound and K-5 Tracer were added per well of the plate. The plate was incubated at 37° C., 5% CO₂ for 2 hours. 3× Complete Substrate plus Inhibitor Solution in Assay Medium (Opti-MEM I Reduced Serum Medium, no phenol red) was prepared by gently mixing a 1:166 dilution of NanoBRET Nano-Glo Substrate plus a 1:500 dilution of Extracellular NanoLuc Inhibitor in Assay Medium. 50 μL of 3× Complete Substrate plus Inhibitor Solution was added to each well of the 96-well plate and incubated for 2-3 minutes at room temperature. Donor emission (450 nm BP) and acceptor emission (600 nm LP) wavelengths were measured using the GloMax Discover System.

Statistical analyses. In general, experiments were repeated independently at least three times unless otherwise specified. Student's t-tests were performed to compare two groups, while multiple-group comparisons were performed using a one-way analysis of variance (ANOVA) test with a post-hoc multiple comparisons test. A p-value less than 0.05 was considered statistically significant. Analyses were done using GraphPad Prism.

### Example 1

### CK2 as a Potent Driver of Glial Inflammation in the Brain

### Anti-Inflammatory Activity of Flavones in Human Glia

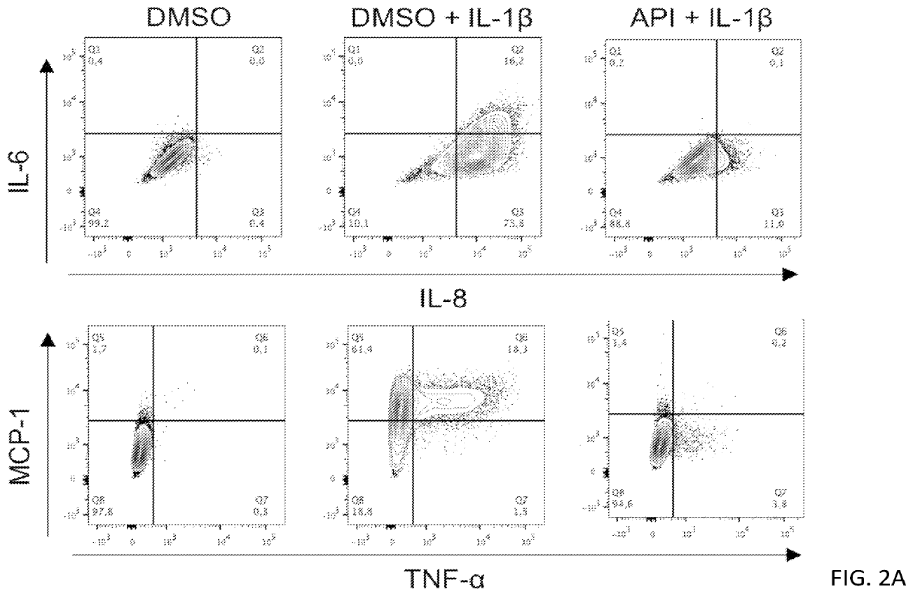

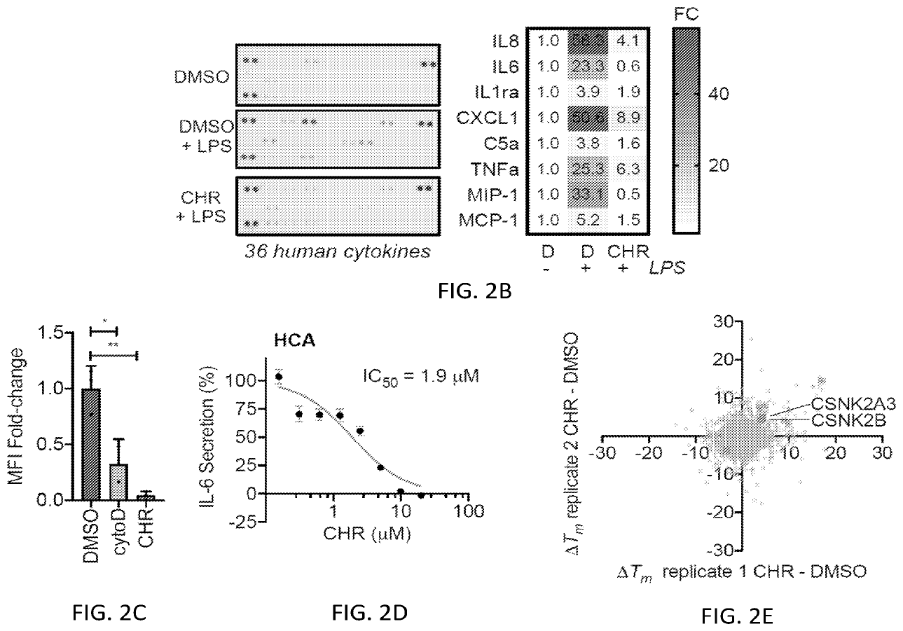

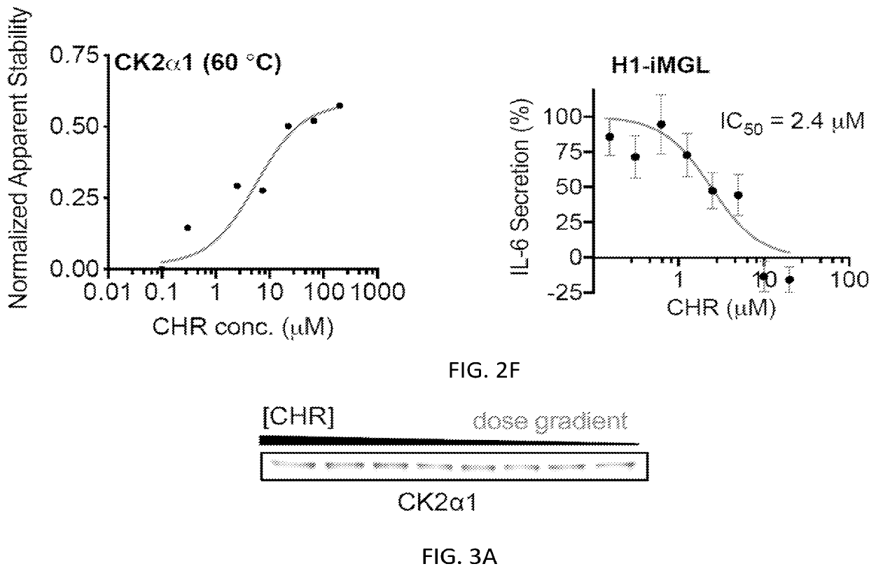

A flow cytometry-based assay was used to assess the anti-inflammatory activity of several natural flavonoid compounds (FIG. 1A) and build corresponding structure-activity relationships. Primary human cerebellar astrocytes (HCA) were co-treated with 20 μM of each compound or vehicle (DMSO), 10 ng/mL IL1-β, and a protein transport inhibitor for 6 hours, and then processed for intracellular staining of pro-inflammatory cytokines IL-6, IL-8, TNF-α, and MCP-1. The presence of a C2-C3 double bond was the predominant determining factor for anti-inflammatory activity. For example, apigenin (API) strongly blocked IL1-β-induced IL-6, IL-8, TNF-α, and MCP-1 cytokine upregulation, unlike its flavanone analogue naringenin (NAR, FIG. 2A and FIG. 1B). Similar divergence in activity was observed with trimethylated apigenin (4′,5,7-trimethoxyflavone, TMF) and its corresponding inactive flavanone analogue 4′,5,7-trimethoxyflavanone (dTMF, FIG. 1C) and chrysoeriol (CHR) versus homoeriodictyol (dCHR, FIGS. 1A, 1D). For subsequent phenotypic testing, active flavones API, TMF, or CHR were used. The effects of CHR treatment on cytokine release in LPS-stimulated human induced pluripotent stem cell (iPSC) derived microglia¹³ was broadly assessed using a panel of 36 cytokines (FIG. 2B). CHR significantly blocked the release of multiple LPS-induced pro-inflammatory cytokines, including interleukins (IL-6, IL-8, IL1ra), chemokines (CXCL1, MCP-1, MIP-1), complement (C5a), and TNF-α (p<0.05, unpaired two-tailed t-tests). Altered phagocytic function of microglia is associated with neurodegenerative disease.¹⁴ CHR blocked microglial phagocytosis of pHrodo E. coli beads (P=0.0015, FIG. 2C, compare to phagocytosis inhibitor cytochalasin D¹⁵). The anti-inflammatory effects of CHR (FIG. 2D) and TMF (FIG. 1C) were dose-dependent in astrocytes and microglia. There was no cytotoxicity associated with flavone treatment (FIG. 1E).

### Target Identification by Thermal Shift Proteome Profiling (TPP) Reveals CK2 as Flavone Target

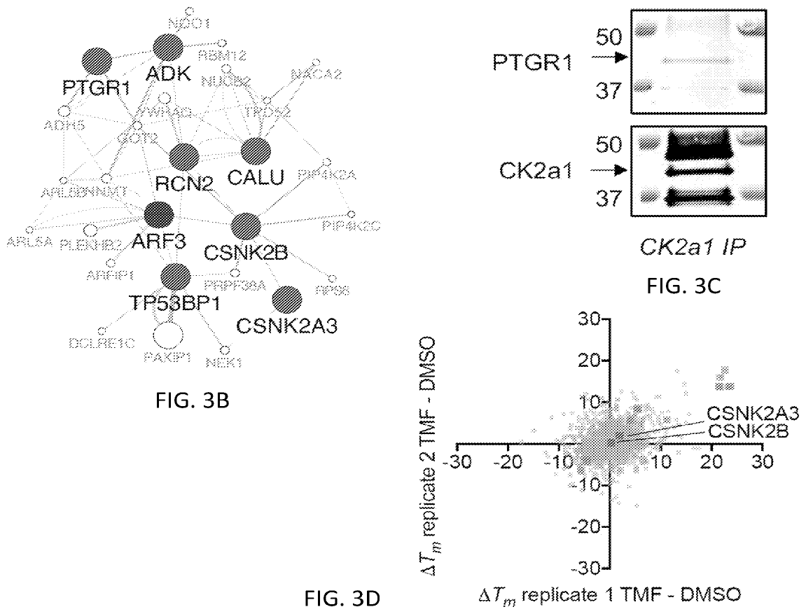

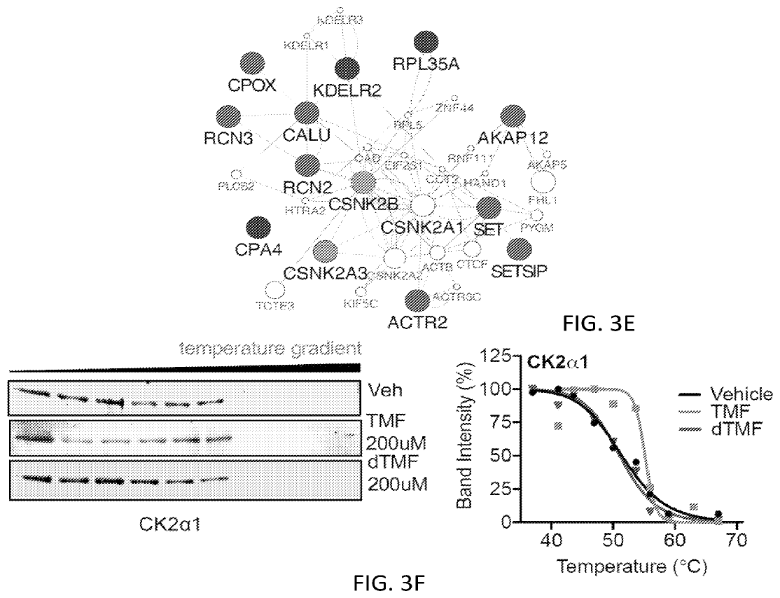

Next, an unbiased proteomics approach was undertaken to identify cellular targets of flavones in inflamed astrocytes. Thermal shift proteome profiling (TPP) identifies drug target engagement in cells by measuring ligand stabilization (or destabilization) of bound proteins against heat denaturation^{16,17}. In this assay, stabilized proteins can be either direct or indirect targets, while destabilized proteins are indirect targets. Lysates were extracted from IL1-β-treated human iPSC-derived astrocytes, and treated with CHR or DMSO for 30 minutes, and TPP was performed using LC-MS/MS with TMT10 labeling. Eight proteins with a significant thermal shift (ΔTₘ) reproducible in two independent experiments were identified (FIG. 2E). Two of the hits, CSNK2A3 (CK2α1P) and CSNK2B (CK2β), constitute components of the CK2 holenzyme. CK2 is a protein kinase that is catalytically active either as a heterotetrameric complex composed of two alpha and two beta subunits or an alpha monomer¹⁸. CSNK2A3 is nearly identical (99% cDNA homology¹⁹) to CSNK2A1, while CSNK2A2 shares 77% identity with CSNK2A1. CSNK2A1 peptides were not detected by mass spectrometry in the TPP experiment. CK2α1 was confirmed to be dose-dependently stabilized by CHR in an isothermal dose response thermal shift assay followed by Western blotting (FIG. 2F, FIG. 3A).

Network analysis of the other identified TPP hits (ADK, PTGR1, TP53BP1, CALU, RCN2, ARF3) show high connectivity with each other and CK2 (FIG. 3B). ARF3 showed thermal destabilization indicative of an indirect hit. ADK and TP53BP1 physically interact with CK2^{20,21}, CALU²² is a known CK2 substrate, and RCN2 is a likely CK2 substrate^{22,23}. Co-immunoprecipitation was performed with CK2α1 and showed that it associates with PTGR1 (FIG. 3C).

The TPP experiment was repeated with another flavone compound, TMF. Eleven proteins were found with a significant thermal shift reproducible in two independent experiments (FIG. 3D). CSNK2A3 and CSNK2B were stabilized by TMF, but not enough to pass the stringent statistical significance requirements. Network analysis of the 11 hits and all 4 CK2 subunits, however, shows high connectivity (FIG. 3E). Among the 8 stabilized proteins, 6 are known CK2 substrates^{22,24} (RCN1, RCN3, SET and its paralogue SETSIP, CALU, AKAP12) and ACTR2 is a known interactor^{20,21}. CK2α1 was validated as a target of TMF (but not inactive flavanone dTMF) in astrocytes in a thermal shift assay followed by Western blotting (FIG. 3F).

Flavones have previously been shown to inhibit CK2 in vitro^{25,30} and in cancer cells^{31,32}, with existing co-crystal structures of flavone derivatives and CK2 reflecting protein binding as ATP-competitive inhibitors^{26,33}. Furthermore, CK2 is known to have roles in inflammation³⁴⁻³⁷. Overall, this data indicated CK2 as the relevant target to pursue for further validation.

### Validation of CK2 as a Potent Anti-Inflammatory Target in Astrocytes

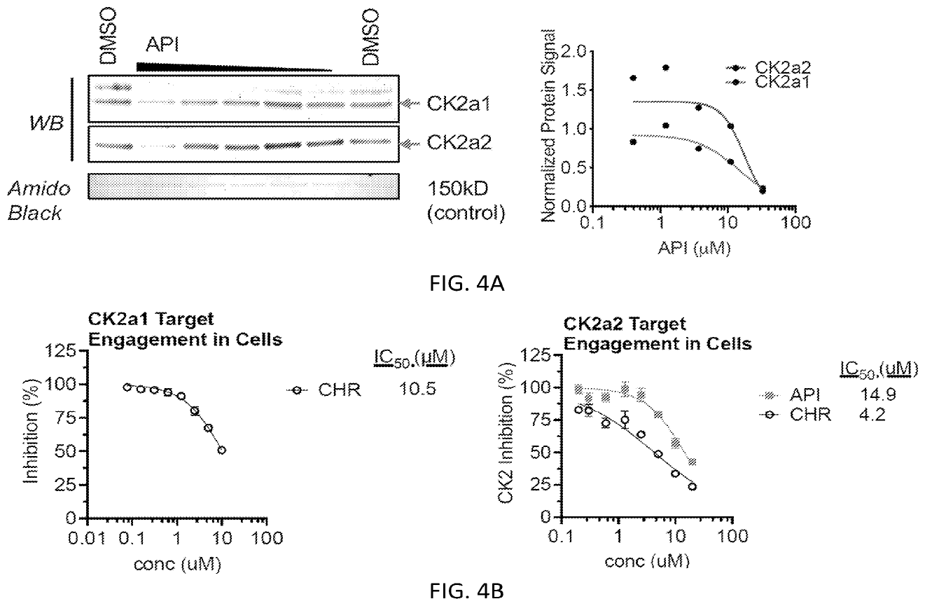

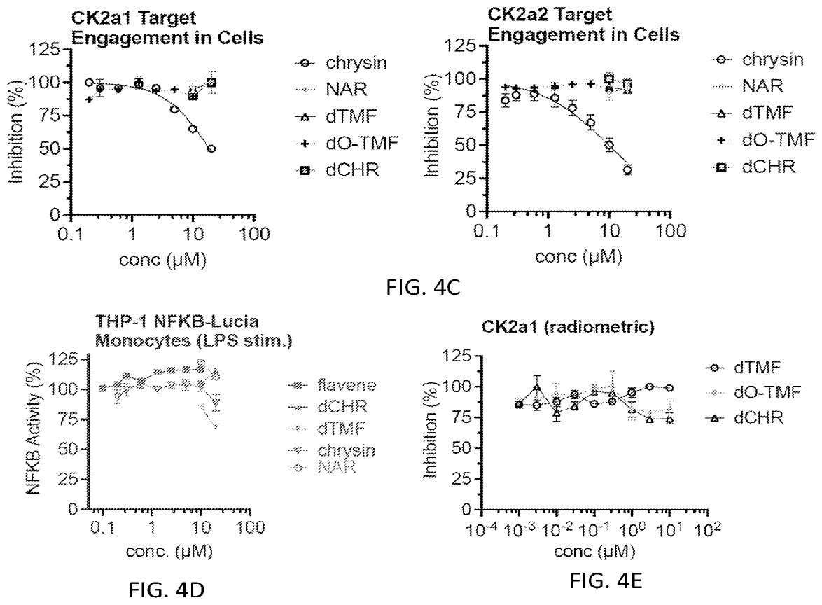

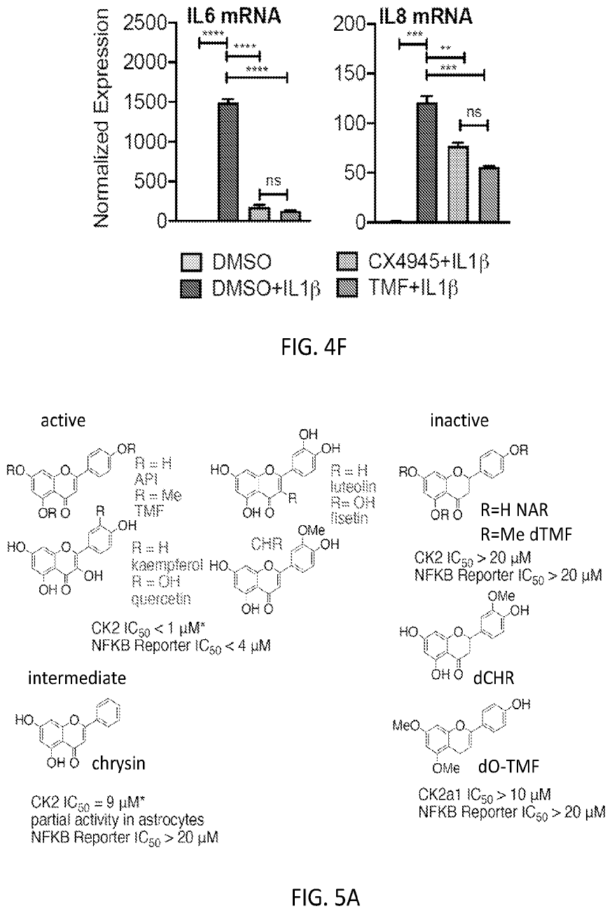

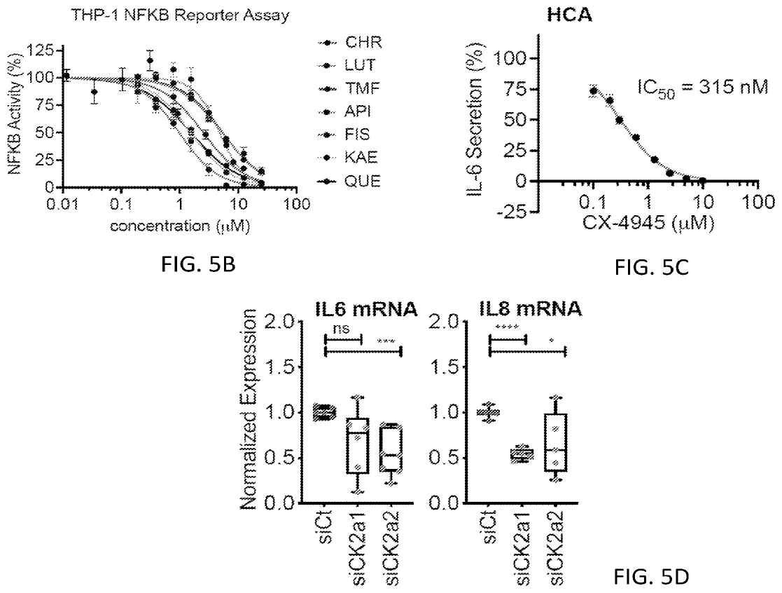

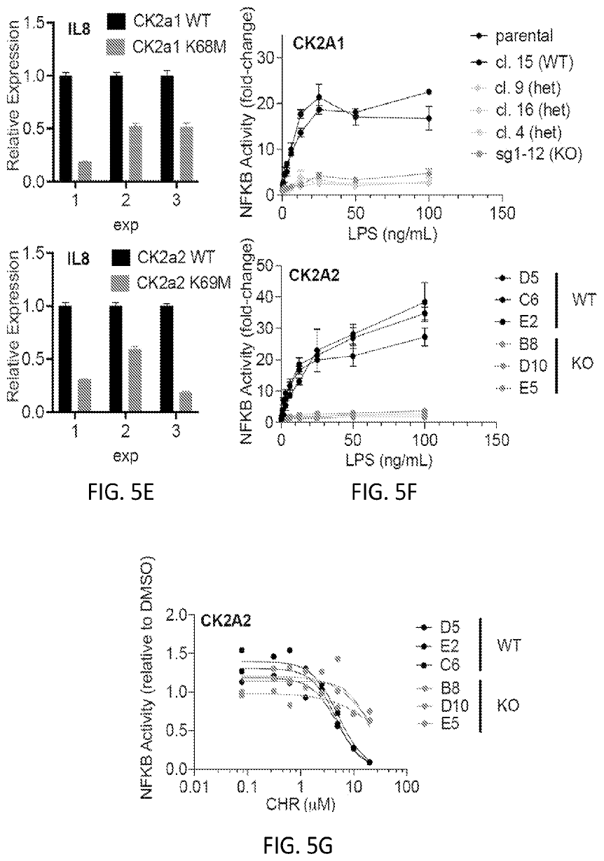

It was verified that flavones directly inhibit CK2 in cells via additional orthogonal methods. In astrocytes, it was found that API displaces a desthiobiotin-ATP probe from both CK2α1 and CK2α2 (FIG. 4A). CK2α1/CK2α2 target engagement of API (IC₅₀=14.9 μM CK2α2) and CHR (IC₅₀=4.2 μM CK2α2, 10.5 μM CK2a1) was also confirmed in cells via NanoBRET³⁸ (Extended Data FIG. 3B). Revisiting the structure-activity studies in the context of CK2 inhibition, it was found that anti-inflammatory activity in astrocytes correlated well with CK2 inhibitory activity (FIG. 5A). API, quercetin, fisetin, luteolin, kaempferol, and CHR possess IC₅₀ values ranging from 80-800 nM^{26,29} as well as strong suppressive activity against at least 3 of 4 cytokines measured (FIGS. 1A, 1C) and strong activity (IC₅₀<4 μM) in THP-1 monocyte NF-κB-Luciferase reporter cells (FIG. 5B). Chrysin possesses a CK2 IC₅₀ value of 9 μM²⁶ and shows partial CK2α1/CK2α2 inhibition in cells (FIG. 4C), with intermediate anti-inflammatory activity exhibiting as variable suppression of 2 cytokines and no activity in THP-1 monocyte NF-κB-Luciferase reporter cells (FIGS. 1A, 4D). Flavanones missing the key C2-C3 double bond identified earlier were completely inactive both as CK2 inhibitors and anti-inflammatory agents (FIGS. 4C-4E, NAR, dTMF, dCHR, IC₅₀>10 μM).

It was determined whether a structurally unrelated CK2 inhibitor could also block inflammation in astrocytes stimulated with IL1-β. Treatment with CX-4945, a potent synthetic CK2 inhibitor currently in clinical trials for cancer, effectively blocked secretion of IL-6 after 5 hours (IC₅₀=315 nM, FIG. 5C). CX-4945³⁹ also successfully blocked expression of IL6 and IL8 mRNA in astrocytes to a similar extent as TMF (FIG. 4F). CX-4945 and CK2 inhibitor TBB have been previously shown to reduce secretion of IL-6 and MCP1 in primary astrocytes.⁴⁰ Recently, Mishra et al. also reported that CX-4945 and SGC-CK2-1, a selective chemical probe for CK2, blocks 1L6 and IL8 expression in iPSC-derived microglia.⁴¹

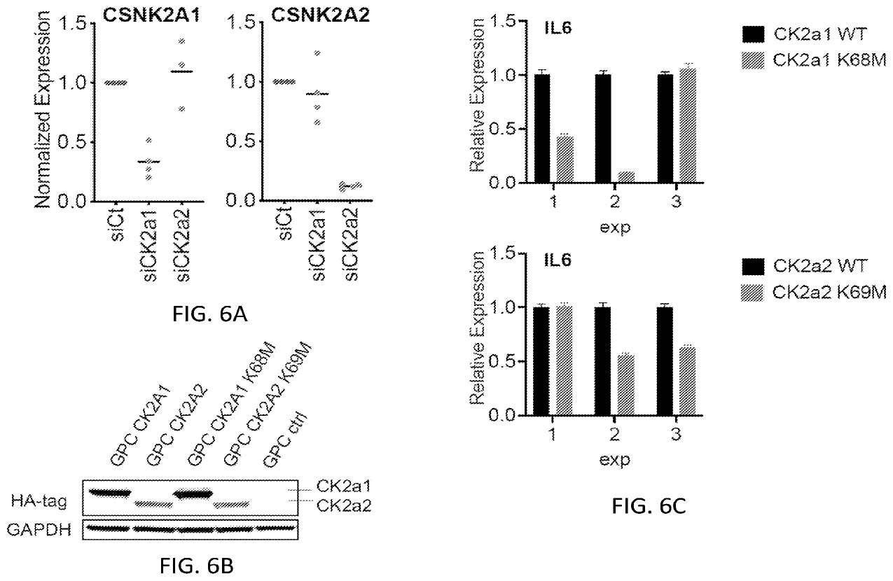

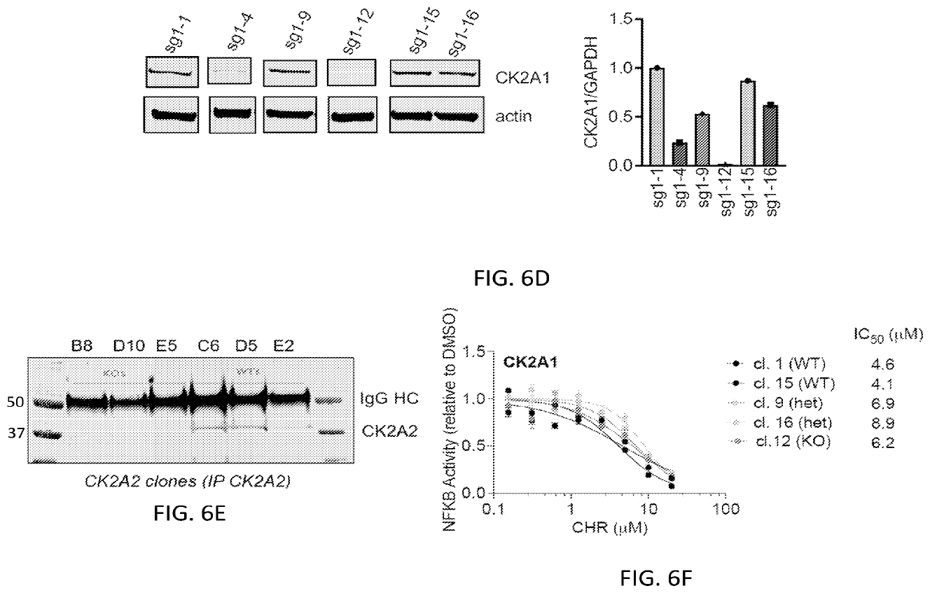

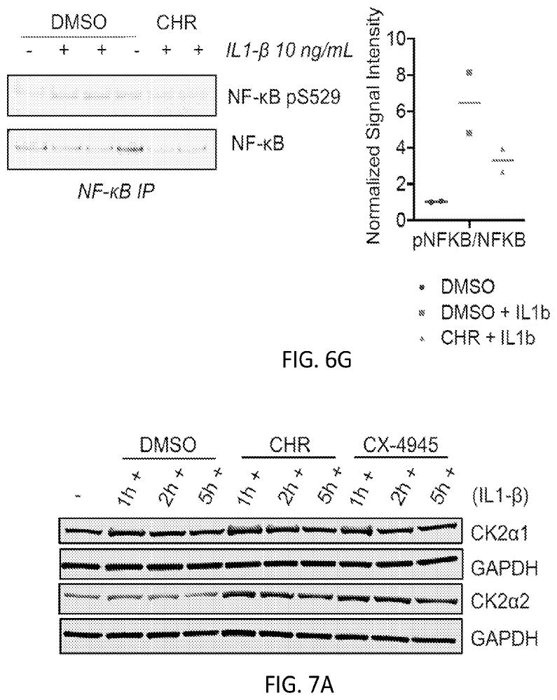

It was determined whether genetic perturbation of CK2 could phenocopy the effects of pharmacological inhibition observed with flavones. siRNA knockdown of individual CK2 paralogues CK2α1 or CK2α2 was performed in human primary astrocytes. After 3 days, cells were treated with IL-1β for 5 hours and RNA was extracted to assess cytokine levels. It was observed that knockdown of either CK2α1 or CK2α2 reduced the ability of IL-1β to induce upregulation of pro-inflammatory cytokines IL6 and IL8 (FIGS. 5D, 6A).

To further dissect the individual roles of the two catalytic forms, CRISPR-Cas9-mediated knockout of CK2A1 or CK2A2 in THP-1 monocyte NF-κB-Luciferase reporter cells and isolated clonal lines were prepared to measure their response to inflammatory stimulation and anti-inflammatory activity of CHR. Isolating homozygous CK2α1 clones proved difficult, highlighting the importance of this isoform for THP-1 cancer cell survival.⁴² Overall, knockout of CK2α1 or CK2α2 reduced the ability of IL1β to induce inflammation in these cells (FIG. 5F and FIGS. 6D, 6E). However, only CK2α2 knockouts became significantly resistant to the anti-inflammatory effects of CHR, highlighting CK2α2 as the primary driver of inflammation (FIGS. 5G, 6F).

Correspondingly, it was determined whether overexpression of a “kinase-dead” form of CK2 could affect inflammatory induction in astrocytes. Glial progenitor cells were transduced with an LV-CK2α1 or CK2α2 wild-type (WT) or single point mutant (K68M and K69M, respectively)⁴³, differentiated for 4 weeks to produce mature astrocytes, then stimulated with IL1-β for 5 hours. CK2α1-K68M significantly abrogated inflammation relative to WT, and a similar but non-significant trend for CK2α2-K69M versus its WT (FIGS. 5D, 6B, 6C).

Overall, these data indicate that CK2 is the relevant cellular target of anti-inflammatory flavones, and that CK2 is an important upstream pro-inflammatory regulator in astrocytes.

### CK2 Activates NF-κB in Response to Inflammatory Stimuli in Astrocytes

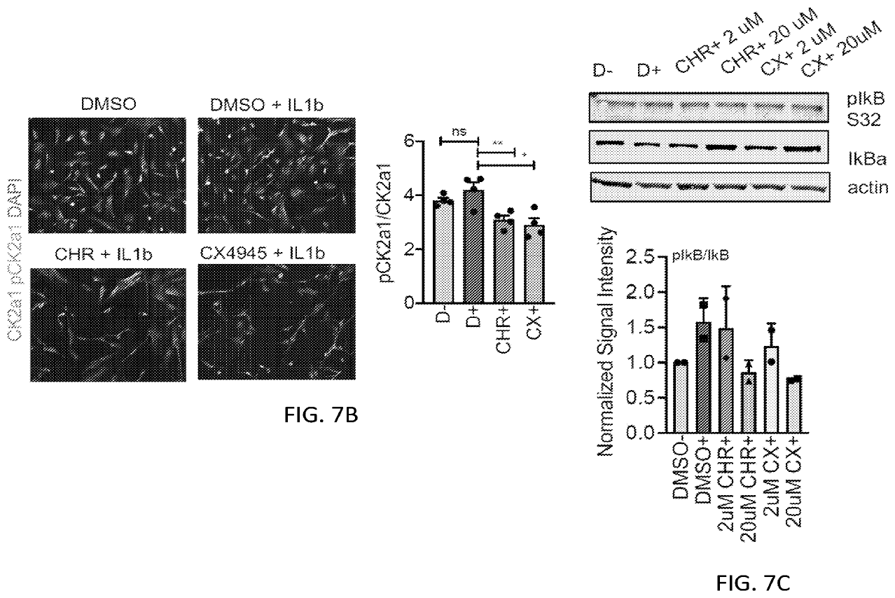

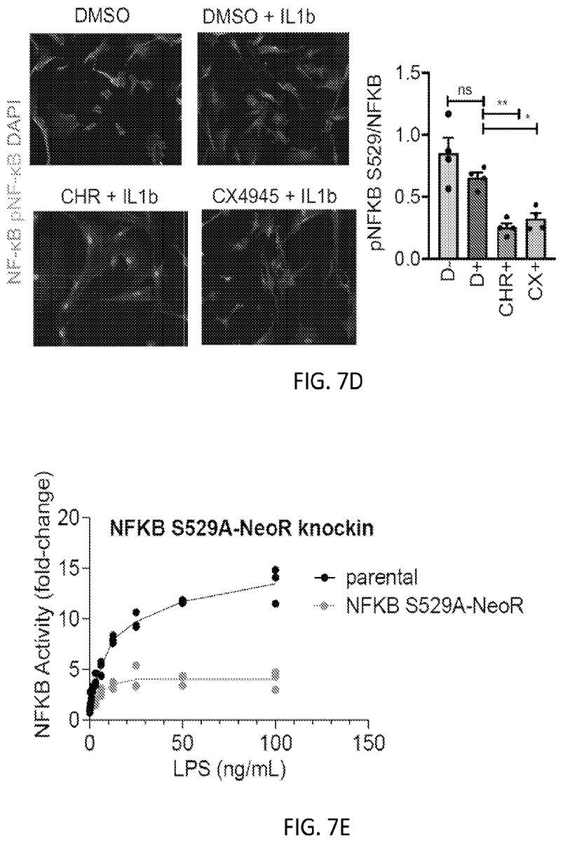

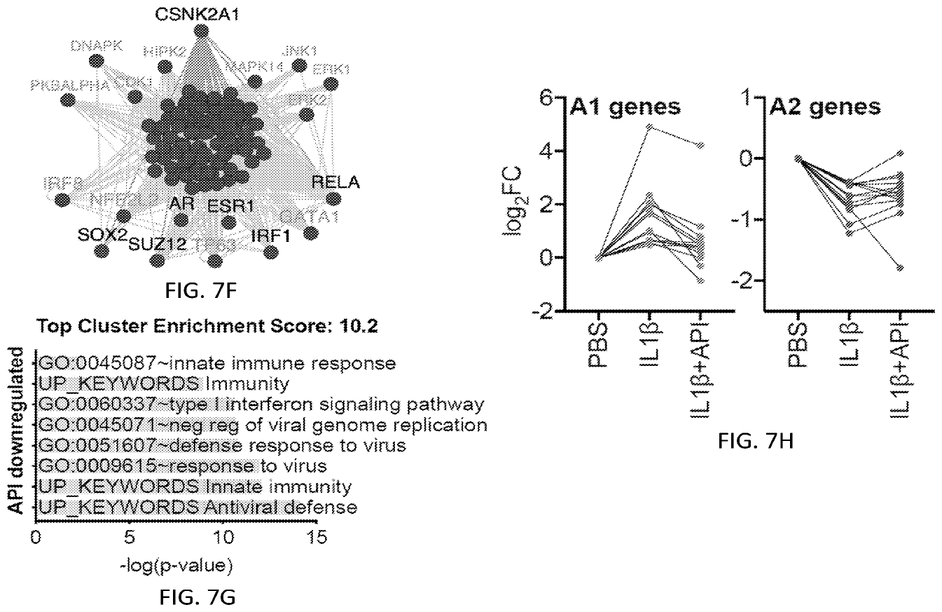

Others have reported that CK2 protein levels increase in macrophages and certain cancer cell lines respond to pro-inflammatory stimuli like LPS and TNF-α^{34,36}. A similar increase in CK2 levels was observed in human primary astrocytes stimulated with IL1-β, with CK2α1 and CK2α2 peaking at 1 h after induction and reduced to baseline with CHR and CX-4945 treatment after 5 hours (FIG. 7A). At this timepoint, CHR and CX-4945-mediated reduction of CK2 pY255 (a marker of CK2 activation⁴⁴) relative to total CK2α1 in astrocytes was observed by immunofluorescence (FIG. 7B). This indicates that CK2 is an immune-responsive kinase in astrocytes.

Several proteins involved in the innate immune response have been characterized as substrates of CK2, including IκB S32⁴⁵ and NF-κB S529⁴⁶. NF-κB is a master transcriptional regulator of the immune response, downstream of a broad repertoire of exogenous stimuli, including Toll-like receptors, IL-1R, and TNFR. IκBα is a negative feedback regulator and acts as an inhibitor of the NF-κB program by sequestering NF-κB in the cytoplasm. IκBα S32 phosphorylation is necessary for degradation of IκBα by pro-inflammatory stimuli.⁴⁵ IκBα was downregulated in inflamed astrocytes after 6 hours, but CK2 inhibitors CHR and CX-4945 dose-dependently boosted IκBα levels while phosphorylated IκB S32 remained unchanged (FIG. 7C). Overall, this is consistent with CK2 acting downstream of IL1-3 to phosphorylate IxB S32, with CK2 inhibition leading to a reduction of pIκB and subsequent stabilization of total IκBα.

To investigate the potential effects of CK2 activity on NF-κB, NF-κB and NF-κB pS529 was measured by immunofluorescence in activated astrocytes. After 5 hours, both CHR and CX-4945 reduced pNF-κB nuclear intensity relative to total NF-κB (FIG. 7D). At the intermediate timepoint of 2 h post-induction with IL1-β, an increase in pNF-κB relative to NF-κB was detected via immunoprecipitation (IP) followed by immunoblotting; this increase in phosphorylated pNF-κB species was abrogated by CHR co-treatment (FIG. 6F). To further study the effects of CK2-dependent NF-κB S529 phosphorylation, a phosphodeficient mutation S529A was knocked in along with the neomycin resistance gene into the endogenous NF-κB locus in THP-I NF-κB-Luciferase cells. The S529A mutation diminished NF-κB activation in response to LPS treatment, phenocopying CK2 inhibition, knockdown, or knockout (FIG. 7E).

These findings correlate the upregulation of CK2 catalytic subunits (FIG. 7A) and the anti-inflammatory activity of CK2 inhibition observed at this timepoint (FIGS. 2D, 5C) with NF-κB activation. Overall, these results reflect the dynamic regulation of NF-κB by CK2 and indicate that CK2 plays an important role in sustaining NF-κB-driven immune response.

### CK2 Kinase Activity Sustains an Inflammatory Transcriptional Program

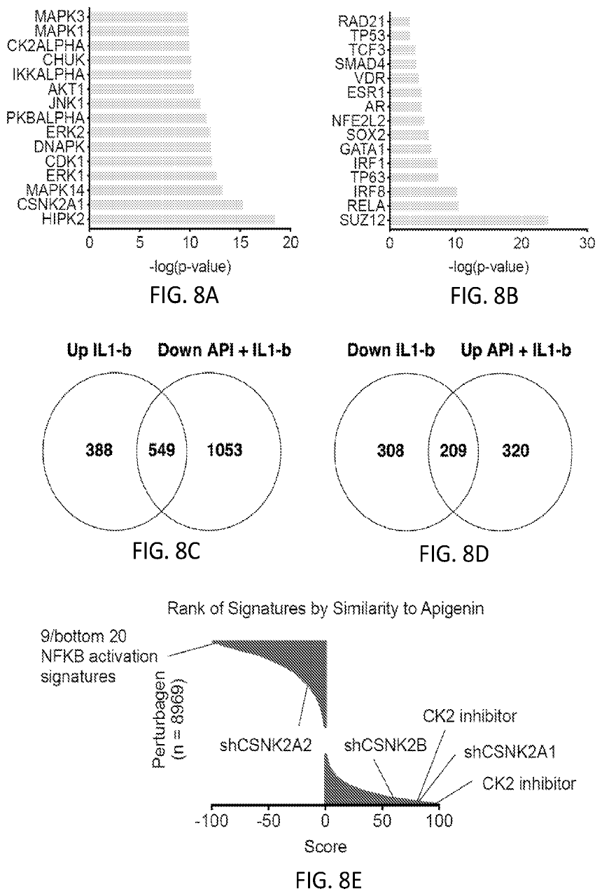

To further investigate the mechanistic role of CK2 in driving inflammation in glia, RNA sequencing (RNA-seq) was performed in human primary astrocytes stimulated with IL1-β and API or vehicle for 5 h. Differential expression (DE) analysis showed 533 genes downregulated and 938 genes upregulated upon activation (P<0.05, FC>2). This inflammatory signature was strongly enriched in genes regulated by CK2, as identified by Kinase Enrichment Analysis⁴⁷ (KEA, P=5.8×10⁻¹⁶ and P=1.19×10⁻¹⁰, FIG. 8A). Indeed, network analysis shows that CK2 plays a central role in regulating many of the downstream enriched transcription factor (TF) modules, including RELA/NF-κB, IRF1, and polycomb repressive complex subunit SUZ12 (FIGS. 7F, 8B). IRF1 is another key TF of innate immunity⁴⁸ and SUZ12/EZH2 have been shown to regulate NF-κB target gene expression⁴⁹.

Next, gene expression of cells treated with API+IL1-β was compared to IL1-β alone, and it was found that 531 genes were upregulated and 1603 genes were downregulated (P<0.05, |log 2FC|>1). Notably, API reversed a significant fraction of the gene expression changes induced in activated astrocytes (58% of the genes upregulated by IL1-β, P=10⁻¹⁰³⁷, FIG. 8C; and 40% of the genes downregulated by IL1-l, P=2.7×10⁻²²⁶, FIG. 8D). Among all the API-downregulated genes, the top cluster of enriched terms by functional gene annotation analysis was related to the innate immune response (FIG. 7G). Querying DE genes following API treatment in the CMap database⁵⁰, multiple CK2 inhibitor and CK2 knockdown signatures exhibited high similarity scores (FIG. 8E). In contrast, nine out of the top 20 most dissimilar signatures in the CMap database related to NF-κB activation. These results indicate the CK2 inhibition effectively attenuates the inflammatory response in astrocytes.

It was determined whether CK2 inhibition modulates the A1/A2 polarization of reactive astrocytes. A1 astrocytes are neurotoxic, forming as a result of chronic inflammation, acute CNS injury, and neurodegenerative diseases, while A2 astrocytes are neuroprotective, promoting tissue repair following cerebral ischemia^{51,52}. Of the 14 A1 genes significantly upregulated by IL1-β in primary astrocytes, 10 of them were significantly downregulated by API (P<0.05, two-tailed t-tests, FIG. 7H). Similarly, of the 13 A2 genes downregulated by IL1-β, 12 were upregulated by API (P<0.05, two-tailed t-tests, FIG. 7G). This shows that CK2 inhibitors suppress the gene expression program underlying the neurotoxic astrocyte phenotype and promote a neuroprotective astrocyte-like transcriptional profile.

Taken together, these data demonstrate that CK2 is a potent master regulator of inflammation in glia upstream of NF-κB. At the transcriptional level, CK2 maintains the polarization of reactive astrocytes towards pro-inflammatory, neurotoxic pathways at the expense of a neuroprotective response. Thus, CK2 inhibition effectively reverses the A1/A2 polarization of inflamed astrocytes by restraining tissue-damaging and promoting tissue-repair transcriptional programs.

### CK2 Activity is Upregulated in Alzheimer's Disease and Contributes to a Hyperinflammatory Astrocyte Phenotype

Having established that CK2 regulates neuroinflammation in human glia, it was determined whether neurodegenerative diseases such as Alzheimer's disease (AD) exhibit dysregulated CK2 activity, as astrogliosis and chronic inflammation underlie pathogenesis in AD. There is some evidence of dysregulation of CK2 in neurodegenerative diseases. Higher CK2 levels were observed in astrocytes in the cortex and hippocampus of AD patients, with localization near amyloid plaques.⁴⁰ Notably, CK2 overexpression causes cognitive decline in wildtype mice, and elevated CK2 activity is observed in both 3×Tg and APP/PS1 mouse models from 3 to 6 months of age.⁵³

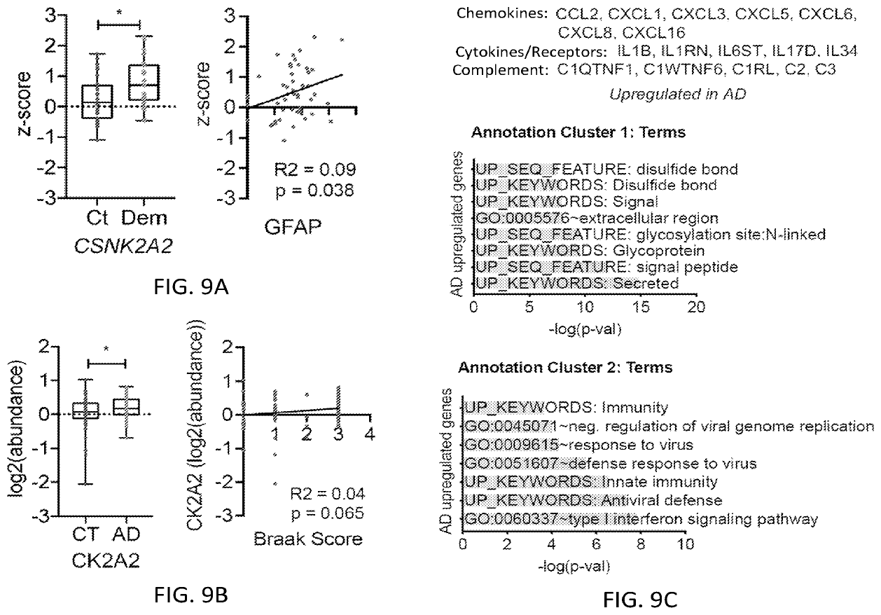

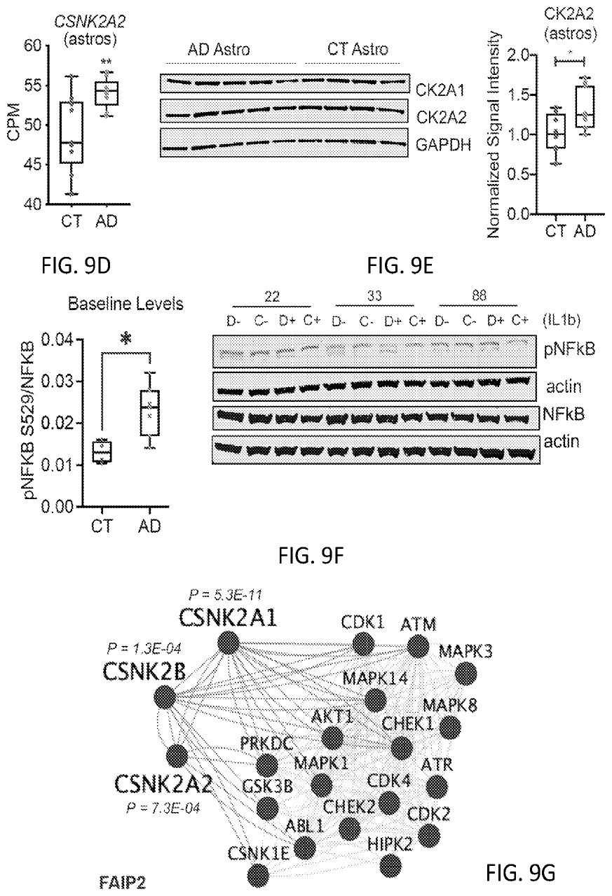

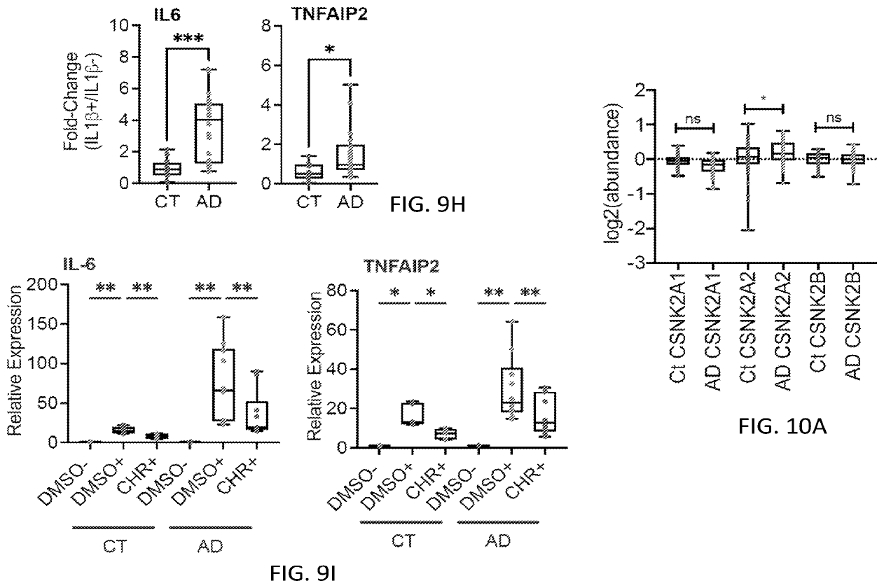

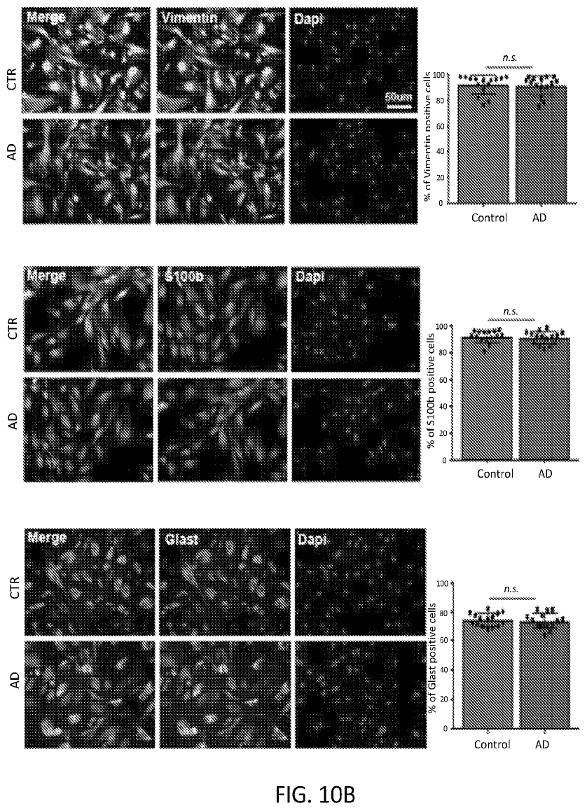

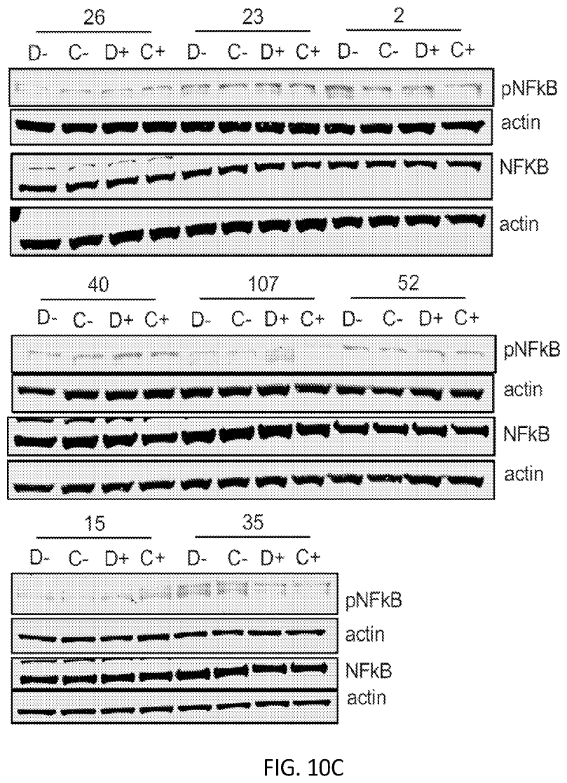

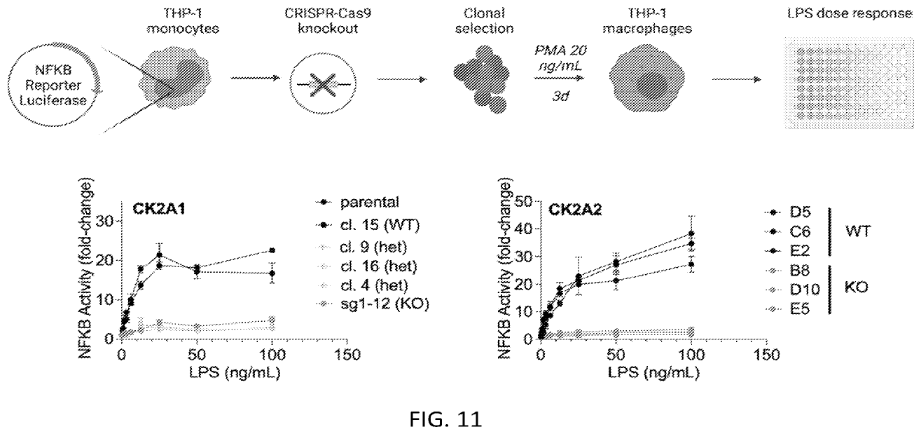

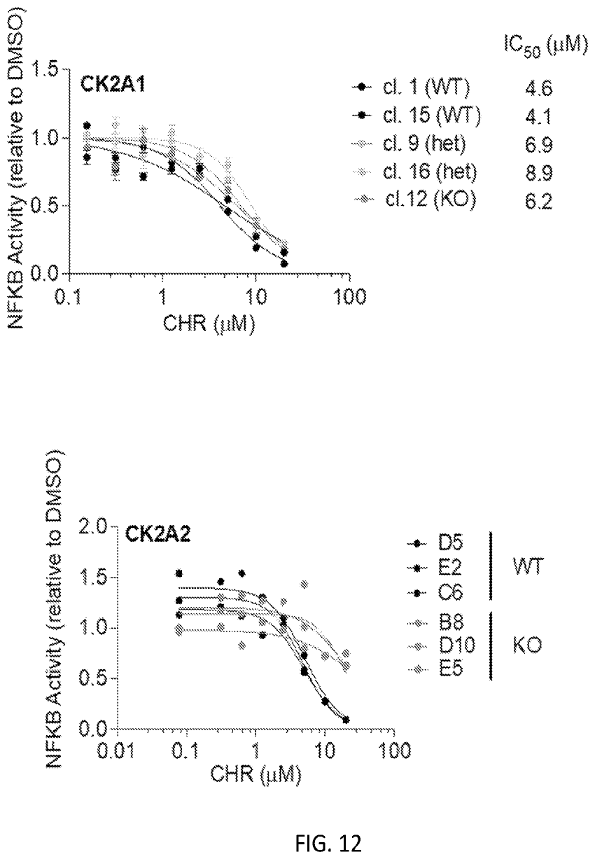

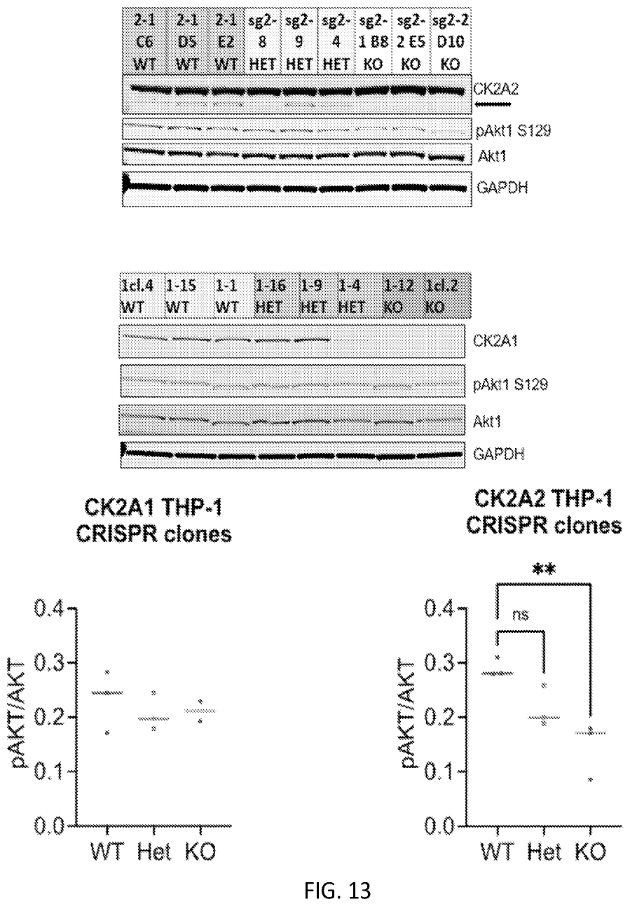

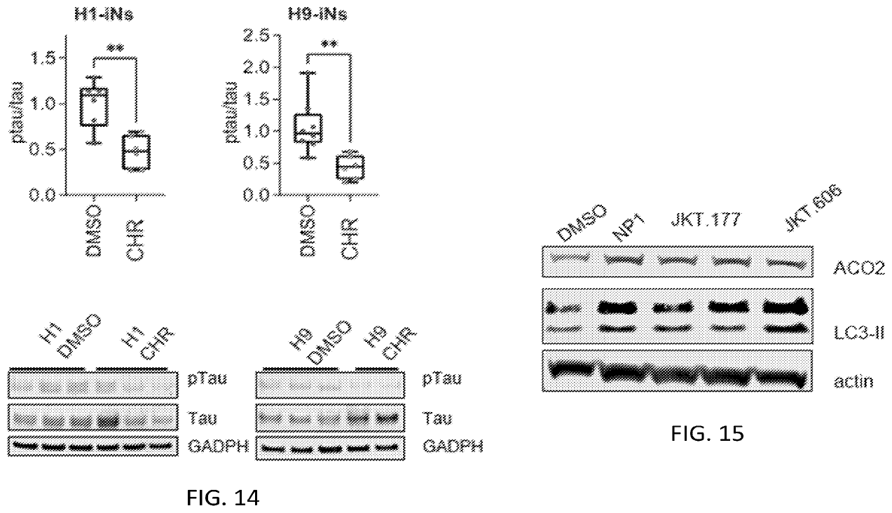

CK2 dysregulation was examined in human AD models. Evaluation of the transcriptome profiling data from the Aging, Dementia, and TBI RNA-seq study demonstrated that CSNK2A2 was significantly upregulated in dementia patients compared to healthy controls (P=0.024, two-tailed t-test, FIG. 9B), and this correlated significantly with GFAP expression, a marker of astrogliosis (P=0.038, R²=0.09). Evaluation of the protein expression data from the UPP Proteomics Study confirmed that CK2α2 protein was also upregulated in AD patients compared to controls, whereas CK2α1 and CK20 were expressed at similar levels (P=0.015, one-way ANOVA with Sidak's post-hoc test, FIGS. 9C, 10A), trending towards significant positive correlation with Braak scores, a marker of disease burden in AD (P=0.065, R²=0.04). Interestingly, CK2α2 expression is enriched in the brain relative to other tissues, unlike CK2α1 which is uniformly expressed throughout tissues⁵⁴. Supporting these findings, Marshall et al. recently reported that CK2 levels in AD hippocampi correlate inversely with MMSE scores, a marker of cognitive decline.⁵⁵

Using a cohort of 7 AD and 5 cognitively normal age-matched control iPSCs, patient-derived astrocytes were generated and it was determined whether these observations could be recapitulated in vitro using this model (FIG. 10B). RNA-seq and differential gene expression analysis was performed with a subset of 3 controls and 3 AD astrocyte lines. 572 upregulated genes and 804 downregulated genes in AD versus CT (FDR<0.05, FC>1.6) were observed. Gene ontology analysis using ClueGO and DAVID showed evidence of baseline inflammatory activation in AD astrocytes, with enriched terms including secreted proteins, innate immunity, and interferon signaling (FIG. 9C). CSNK2A2 was significantly upregulated in AD patient-derived astrocytes compared to controls (FIG. 9C). CK2α2 protein levels were also significantly and specifically elevated in AD patient-derived astrocytes, and this was associated with downstream elevated phosphorylated NF-κB that could be reduced with CHR treatment (FIGS. 9E, 9F, 10C). Transcriptionally, KEA showed that all three CK2 subunits were among the top 20 enriched kinase hubs regulating AD-upregulated genes (FIG. 9G). These data support a role for CK2, especially isoform CK2α2, in driving a chronic inflammatory transcriptional program in AD astrocytes.

It was determined whether AD astrocytes could have a dysregulated response to inflammatory stimuli in addition to their baseline inflamed state. Indeed, AD astrocytes exhibited a hyperinflammatory response to IL1-β treatment, as demonstrated by the higher magnitude of IL-6 and TNFAIP2 induction compared to controls (FIG. 9H). This hyperinflammatory phenotype was mitigated by CK2 inhibition with CHR, which also reduced NF-κB phosphorylation (FIGS. 9F, 9I).

Taken together, these data indicate that CK2 activity is upregulated in neurodegenerative diseases like AD, and that AD astrocytes exhibit a hyperinflammatory state due in part to CK2 activation. In AD patient-derived astrocytes, CK2 levels correlate with higher induced secretion of inflammatory cytokines like IL-6 that could be abrogated via small molecule CK2 inhibition.

Although several anti-inflammatory drugs are being currently pursued in clinical trials for neurodegenerative disease, specific pathways and master regulators of inflammation in the brain remain to be fully elucidated. Through phenotype-guided studies of natural product activities, it is shown herein that kinase CK2 is a potent upstream regulator of NF-κB-driven innate immunity transcriptional programs. Thus, CK2 inhibition is a novel therapeutic avenue for patients with neurodegenerative diseases.
1. Skaper, S. D., Facci, L., Zusso, M. & Giusti, P. An Inflammation-Centric View of Neurological Disease: Beyond the Neuron. Frontiers in Cellular Neuroscience 12, 325 (2018).
2. Frank-Cannon, T. C., Alto, L. T., McAlpine, F. E. & Tansey, M. G. Does neuroinflammation fan the flame in neurodegenerative diseases? Molecular Neurodegeneration 4, 47 (2009).
3. Glass, C. K., Saijo, K., Winner, B., Marchetto, M. C. & Gage, F. H. Mechanisms Underlying Inflammation in Neurodegeneration. CELL 140, 918-934 (2010).
4. Becher, B., Spath, S. & Goverman, J. Cytokine networks in neuroinflammation. Nature Reviews Immunology 17, 49-59 (2017).
5. Vainchtein, I. D. & Molofsky, A. V. Astrocytes and Microglia: In Sickness and in Health. Trends in Neurosciences 1-11 (2020) doi:10.1016/j.tins.2020.01.003.
6. Pluvinage, J. V. & Wyss-Coray, T. Systemic factors as mediators of brain homeostasis, ageing and neurodegeneration. Nat Rev Neurosci 21, 93-102 (2020).
7. Zhou, X., Wang, F., Zhou, R., Song, X. & Xie, M. Apigenin: A current review on its beneficial biological activities. Journal of Food Biochemistry 41, e12376 (2017).
8. Mendes, L. F., Gaspar, V. M., Conde, T. A., Mano, J. F. & Duarte, I. F. Flavonoid-mediated immunomodulation of human macrophages involves key metabolites and metabolic pathways. Scientific Reports 9, 1-10 (2019).
9. Chen, L. et al. Intracellular signaling pathways of inflammation modulated by dietary flavonoids: The most recent evidence. Critical Reviews in Food Science and Nutrition 63, 1-17 (2017).
10. Swinney, D. C. Phenotypic vs. Target-Based Drug Discovery for First-in-Class Medicines. Clinical Pharmacology \& Therapeutics 93, 299-301 (2013).
11. Muschietti, L. V., Ulloa, J. L. & Redko, F. D. The Role of Flavonoids as Modulators of Inflammation and on Cell Signaling Pathways. in vol. 196 159-208 (2018).
12. Mrvová, N., Škandfk, M., Bezek, Š. & Račková, L. Protective Effect of Semisynthetic and Natural Flavonoid on Aged Rat Microglia-enriched Cultures. Neurotoxicity Research 147, 1-15 (2019).
13. Abud, E. M. et al. iPSC-Derived Human Microglia-like Cells to Study Neurological Diseases. Neuron 94, 278-293.e9 (2017).
14. Janda, E., Boi, L. & Carta, A. R. Microglial Phagocytosis and Its Regulation: A Therapeutic Target in Parkinson's Disease? Frontiers in Molecular Neuroscience 11, 191 (2018).
15. Zahn, J. von, Möller, T., Kettenmann, H. & Nolte, C. Microglial phagocytosis is modulated by pro- and anti-inflammatory cytokines. Neuroreport 8, 3851-3856 (1997).
16. Reinhard, F. B. M. et al. Thermal proteome profiling monitors ligand interactions with cellular membrane proteins. Nature Methods 12, 1129-1131 (2015).
17. Franken, H. et al. Thermal proteome profiling for unbiased identification of direct and indirect drug targets using multiplexed quantitative mass spectrometry. Nature protocols 10, 1567-1593 (2015).
18. Filhol, O., Martiel, J.-L. & Cochet, C. Protein kinase CK2: a new view of an old molecular complex. EMBO reports 5, 351-355 (2004).
19. Wirkner, U. et al. Human casein kinase II subunit a: Sequence of a processed (pseudo) gene and its localization on chromosome 11. Biochimica et Biophysica Acta (BBA)—Gene Structure and Evpression 1131, 220-222 (1992).
20. Cerami, E. G. et al. Pathway Commons, a web resource for biological pathway data. Nucleic acids research 39, D685-D690 (2010).
21. Rouillard, A. D. et al. harmonizome: a collection of processed datasets gathered to serve and mine knowledge about genes and proteins|Database|Oxford Academic. Database 2016, baw100 (2016).
22. Sugiyama, N., Imamura, H. & Ishihama, Y. Large-scale Discovery of Substrates of the Human Kinome. Scientific Reports 9, 10503 (2019).
23. Goldman, A. et al. The Calcineurin Signaling Network Evolves via Conserved Kinase-Phosphatase Modules that Transcend Substrate Identity. MOLCEL 55, 422-435 (2014).
24. Bian, Y. et al. Global Screening of CK2 Kinase Substrates by an Integrated Phosphoproteomics Workflow. Scientific Reports 3, 681 (2013).
25. Golub, A. G. et al. Structure-based discovery of novel flavonol inhibitors of human protein kinase CK2. Molecular and cellular biochemistry 356, 107-115 (2011).
26. Lolli, G. et al. Inhibition of Protein Kinase CK2 by Flavonoids and Tyrphostins. A Structural Insight. Biochemistry 51, 6097-6107 (2012).
27. Sarno, S. et al. Toward the rational design of protein kinase casein kinase-2 inhibitors. Pharmacology & therapeutics 93, 159-168 (2002).
28. Haidar, S., Jabbour, M., Al-Khayat, M. A., Aichele, D. & Jose, J. Synthesis and biological evaluation of novel 2 (4′-hydroxynaphthyl) chromen-4-one as a CK2 inhibitor. doi:10.1681/ph.2018.7971.
29. Baier, A., Nazaruk, J., Galicka, A. & Szyszka, R. Inhibitory influence of natural flavonoids on human protein kinase CK2 isoforms: effect of the regulatory subunit. Molecular and cellular biochemistry 3, 1-8 (2017).
30. Baier, A., Galicka, A., Nazaruk, J. & Szyszka, R. Selected flavonoid compounds as promising inhibitors of protein kinase CK2α and CK2α′, the catalytic subunits of CK2. Phytochemistry 136, 39-45 (2017).
31. Zhao, M. et al. Apigenin inhibits proliferation and induces apoptosis in human multiple myeloma cells through targeting the trinity of CK2, Cdc37 and Hsp90. Molecular Cancer 10, 104 (2011).
32. Yoshihisa, Y., Andoh, T., Rehman, M. U. & Shimizu, T. The regulation of protein kinase casein kinase II by apigenin is involved in the inhibition of ultraviolet B-induced macrophage migration inhibitory factor-mediated hyperpigmentation. Phytotherapy Research 17, 85 (2019).
33. Niefind, K. et al. Structural Hypervariability of the Two Human Protein Kinase CK2 Catalytic Subunit Paralogs Revealed by Complex Structures with a Flavonol- and a Thieno[2,3-d]pyrimidine-Based Inhibitor. Pharmaceuticals 10, 9-16 (2017).
34. Ampofo, E. et al. Inhibition of protein kinase CK2 suppresses tumor necrosis factor (TNF)-α-induced leukocyte-endothelial cell interaction.—PubMed—NCBI. Biochimica et Biophysica Acta (BBA)—Molecular Basis of Disease 1852, 2123-2136 (2015).
35. Huang, J. et al. Protein kinase CK2α catalytic subunit ameliorates diabetic renal inflammatory fibrosis via NF-κB signaling pathway. Biochemical Pharmacology 132, 102-117 (2017).
36. Singh, N. N. & Ramji, D. P. Protein kinase CK2, an important regulator of the inflammatory response? Journal of Molecular Medicine 86, 887-897 (2008).
37. Gibson, S. A., Yang, W., Yan, Z., Qin, H. & Benveniste, E. N. CK2 Controls Th17 and Regulatory T Cell Differentiation Through Inhibition of FoxO1. J Immunol 201, 383-392 (2018).
38. Vasta, J. D. et al. Quantitative, Wide-Spectrum Kinase Profiling in Live Cells for Assessing the Effect of Cellular ATP on Target Engagement. Cell chemical biology 25, 206-214.el1 (2018).
39. Siddiqui-Jain, A. et al. CX-4945, an orally bioavailable selective inhibitor of protein kinase CK2, inhibits prosurvival and angiogenic signaling and exhibits antitumor eff.—PubMed —NCBI. Cancer Research 70, 10288-10298 (2010).
40. Rosenberger, A. F. N. et al. Increased occurrence of protein kinase CK2 in astrocytes in Alzheimer's disease pathology. Journal of Neuroinflammation 13, 4 (2016).
41. Mishra, S., Kinoshita, C., Axtman, A. D. & Young, J. E. Evaluation of a Selective Chemical Probe Validates That CK2 Mediates Neuroinflammation in a Human Induced Pluripotent Stem Cell-Derived Mircroglial Model. Front Mol Neurosci 15, 824956 (2022).
42. Klink, M. et al. Mechanistic Basis for In Vivo Therapeutic Efficacy of CK2 Inhibitor CX-4945 in Acute Myeloid Leukemia. Cancers 13, 1127 (2021).
43. Penner, C. G., Wang, Z. & Litchfield, D. W. Expression and localization of epitope-tagged protein kinase CK2. Journal of Cellular Biochemistry 64, 525-537 (1997).
44. Donella-Deana, A. et al. Tyrosine phosphorylation of protein kinase CK2 by Src-related tyrosine kinases correlates with increased catalytic activity. Biochemical Journal 372, 841-849 (2003).
45. Taylor, J. A. et al. Serine 32 and serine 36 of IxBa are directly phosphorylated by protein kinase CKII in Vitro. Journal of Molecular Biology 290, 839-850 (1999).
46. Wang, D. & Jr., A. S. B. Activation of Nuclear Factor-κB-dependent Transcription by Tumor Necrosis Factor-a Is Mediated through Phosphorylation of RelA/p65 on Serine 529. Journal of Biological Chemistry 273, 29411-29416 (1998).
47. Chen, E. Y. et al. Expression2Kinases: mRNA profiling linked to multiple upstream regulatory layers. Bioinformatics 28, 105-111 (2012).
48. Platanitis, E. & Decker, T. Regulatory Networks Involving STATs, IRFs, and NFκB in Inflammation. Frontiers in Immunology 9, 771 (2018).
49. Lee, S. T. et al. Context-Specific Regulation of NF-κB Target Gene Expression by EZH2 in Breast Cancers. Molecular Cell 43, 798-810 (2011).
50. Subramanian, A. et al. A Next Generation Connectivity Map: L1000 Platform and the First 1,000,000 Profiles. CELL 171, 1437-1452.e17 (2017).
51. Zamanian, J. L. et al. Genomic Analysis of Reactive Astrogliosis. The Journal of Neuroscience 32, 6391-6410 (2012).
52. Liddelow, S. A. et al. Neurotoxic reactive astrocytes are induced by activated microglia. Nature 541, 481-487 (2017).
53. Zhang, Q. et al. CK2 Phosphorylating I2PP2A/SET Mediates Tau Pathology and Cognitive Impairment. Frontiers in Molecular Neuroscience 11, 1037-13 (2018).
54. Xu, X., Toselli, P. A., Russell, L. D. & Seldin, D. C. Globozoospermia in mice lacking the casein kinase II α′ catalytic subunit. Nature genetics 23, 118-121 (1999).
55. Marshall, C. A. et al. Inhibition of CK2 mitigates Alzheimer's tau pathology by preventing NR2B synaptic mislocalization. Acta Neuropathologica Commun 10, 30 (2022).

### Example 2

### Inducing Anti-Inflammatory Effects by Selective CK2A2 Isoform Inhibition

CK2A2 is significantly upregulated in Alzheimer's disease, while CK2A1 is not. Moreover, Ha et al show that CK2A2 is upregulated in Huntington's disease. In other disease contexts, like Parkinson's, it was found that CK2 isoform levels remain unchanged, but CK2 activity is itself upregulated. As such, it is anticipated that there might be disease-dependent contexts in which isoform-selective CK2 inhibition would be preferred. To investigate this further, CRISPR-Cas9 knockouts in THP-1 NFKB Reporter monocytes were generated and it was found that either CK2A1 or CK2A2 deletion blocked the ability of these cells to activate in response to LPS (FIG. 11). CK2A2 knockouts also exhibited attenuated anti-inflammatory activity in response to CK2 inhibitor treatment. CK2A1 knockout, however, only induced a small right-ward shift in the IL-6 secretion IC₅₀ of CK2 inhibitor CHR (FIG. 12). These results indicate that either CK2A1 or CK2A2 are sufficient to induce inflammation in human cells, but CK2A2 is largely responsible and thus necessary. Moreover, full CK2A1 knockout appeared to be deleterious in THP-1 monocytes, as it was difficult to isolate homozygous CK2A1 knockout clones. This highlights the idea that selective inhibition of CK2A2 over CK2A1 in a therapeutic context is preferred to minimize potential on-target toxicity.

Compounds with improved selectivity for CK2A2 show anti-inflammatory activity in astrocytes (Table 2). Multiple substitution patterns on the flavone scaffold leading to improved selectivity for CK2A2 over CK2A1 in multiple assays were identified. It is notable that a window of selectivity may open or accentuate in cell-based measurements relative to biochemical measurements. The potency and window of selectivity for CK2A2 over CK2A1 exhibited by JKT.464A was striking.

TABLE 2

| | CK2A1 | CK2A2 | |
|---|---|---|---|
| | RAD | RAD | IL6- |
| | IC50 | IC50 | Inhib. |
| Structure | (nM)¹ | (nM) | (μM)² |
| 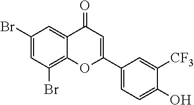 [SMILES: O=c1cc(-c2ccc(O)c(C(F)(F)F)c2)oc2c(Br)cc(Br)cc12] | 609 | 129 | 3 |
| JKT.460A | | | |
| 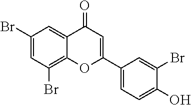 [SMILES: O=c1cc(-c2ccc(O)c(Br)c2)oc2c(Br)cc(Br)cc12] | 19 | 3 | 5 |
| JKT.464A | | | |
| 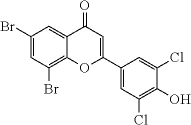 [SMILES: O=c1cc(-c2cc(Cl)c(O)c(Cl)c2)oc2c(Br)cc(Br)cc12] | 134 | 45 | 4.9 |
| JKT.465A | | | |
| 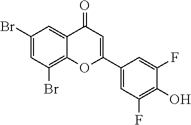 [SMILES: O=c1cc(-c2cc(F)c(O)c(F)c2)oc2c(Br)cc(Br)cc12] | 76 | 32 | >10 |
| JKT.461B | | | |
| 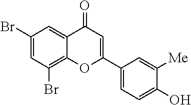 [SMILES: Cc1cc(-c2cc(=O)c3cc(Br)cc(Br)c3o2)ccc1O] | >5000 | 2064 | >10 |
| JKT.475A | | | |
| 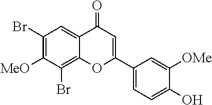 [SMILES: COc1cc(-c2cc(=O)c3cc(Br)c(OC)c(Br)c3o2)ccc1O] | 247 | 49 | 8.6 |
| JKT.706A | | | |
| 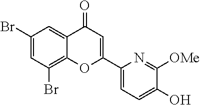 [SMILES: COc1nc(-c2cc(=O)c3cc(Br)cc(Br)c3o2)ccc1O] | 9 | 4 | 3.5 |
| JKT.606A | | | |
| 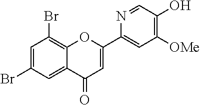 [SMILES: COc1cc(-c2cc(=O)c3cc(Br)cc(Br)c3o2)ncc1O] | 21 | 19 | 6.7 |
| JKT.77A | | | |
| 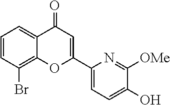 [SMILES: COc1nc(-c2cc(=O)c3cccc(Br)c3o2)ccc1O] | 5 | 8 | 2.0 |
| JKT.666A | | | |
| 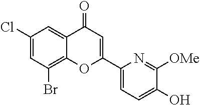 [SMILES: COc1nc(-c2cc(=O)c3cc(Cl)cc(Br)c3o2)ccc1O] | 7 | 5 | 2.9 |
| JKT.669A | | | |
| 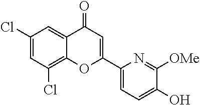 [SMILES: COc1nc(-c2cc(=O)c3cc(Cl)cc(Cl)c3o2)ccc1O] | 17 | 8 | 3.6 |
| JKT.678A | | | |

¹Radiometric kinase assay from Eurofins. Performed in astrocytes with a Promega Lumit IL-6 kit. A series of halogenated flavones was generated and assayed (Table 3).

TABLE 3

| | | | | Aqueous |
|---|---|---|---|---|
| | CK2A1 RAD | CK2A2 RAD | IL6-Inhib. | Solubility |
| Structure | IC50 (nM) | IC50 (nM) | (μM) | (μM) |
| 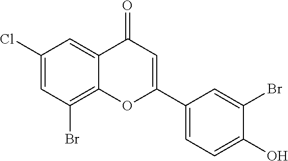 [SMILES: O=c1cc(-c2ccc(O)c(Br)c2)oc2c(Br)cc(Cl)cc12] | 12 | 8 | 4.6 | 35.2 |
| JKT.714A | | | | |
| 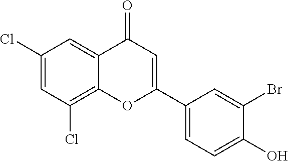 [SMILES: O=c1cc(-c2ccc(O)c(Br)c2)oc2c(Cl)cc(Cl)cc12] | 18 | 16 | 4.1 | 119.8 |
| JKT.715A | | | | |
| 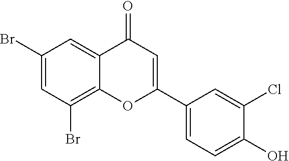 [SMILES: O=c1cc(-c2ccc(O)c(Cl)c2)oc2c(Br)cc(Br)cc12] | 10 | 5 | 2.6 | 19.6 |
| JKT.721A | | | | |
| 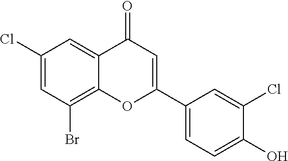 [SMILES: O=c1cc(-c2ccc(O)c(Cl)c2)oc2c(Br)cc(Cl)cc12] | 10 | 7 | 2.2 | 27.5 |
| JKT.722A | | | | |
| 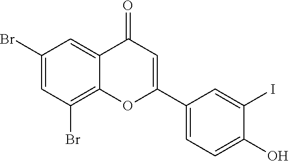 [SMILES: O=c1cc(-c2ccc(O)c(I)c2)oc2c(Br)cc(Br)cc12] | 19 | 7 | 3.8 | 11.2 |
| JKT.724A | | | | |
| 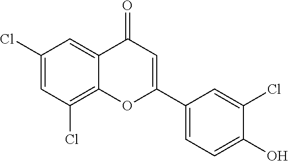 [SMILES: O=c1cc(-c2ccc(O)c(Cl)c2)oc2c(Cl)cc(Cl)cc12] | 15 | 12 | 4.8 | 39.9 |
| JKT.725A | | | | |
| 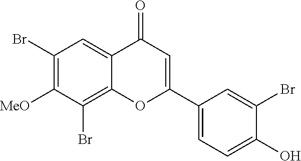 [SMILES: COc1c(Br)cc2c(=O)cc(-c3ccc(O)c(Br)c3)oc2c1Br] | 20 | 11 | 3.0 | >200 |
| JKT.745A | | | | |
| 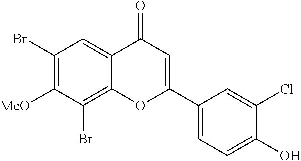 [SMILES: COc1c(Br)cc2c(=O)cc(-c3ccc(O)c(Cl)c3)oc2c1Br] | 24 | 9 | 3.9 | 24.3 |
| JKT.748A | | | | |
| 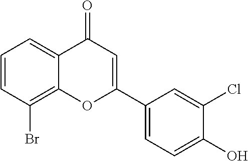 [SMILES: O=c1cc(-c2ccc(O)c(Cl)c2)oc2c(Br)cccc12] | 11 | 18 | 4.8 | 36.5 |
| JKT.749A | | | | |
| 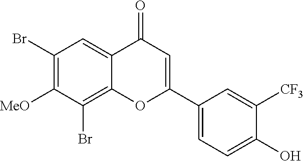 [SMILES: COc1c(Br)cc2c(=O)cc(-c3ccc(O)c(C(F)(F)F)c3)oc2c1Br] | 580 | 466 | 2.2 | 124.4 |
| JKT.750A | | | | |
| 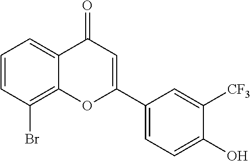 [SMILES: O=c1cc(-c2ccc(O)c(C(F)(F)F)c2)oc2c(Br)cccc12] | 635 | 637 | 5.0 | 4.0 |
| JKT.751A | | | | |
| 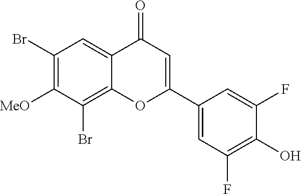 [SMILES: COc1c(Br)cc2c(=O)cc(-c3cc(F)c(O)c(F)c3)oc2c1Br] | >5000 | 1848 | >10 | 109.4 |
| JKT.753A | | | | |
| 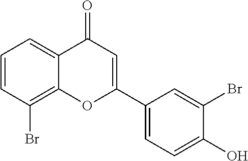 [SMILES: O=c1cc(-c2ccc(O)c(Br)c2)oc2c(Br)cccc12] | 13 | 17 | 3.7 | 5.0 |
| JKT.744B | | | | |
| 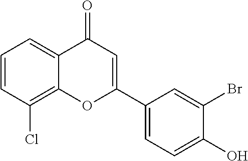 [SMILES: O=c1cc(-c2ccc(O)c(Br)c2)oc2c(Cl)cccc12] | 20 | 21 | 10.3 | 14 |
| JKT.777A | | | | |
| 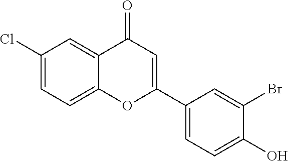 [SMILES: O=c1cc(-c2ccc(O)c(Br)c2)oc2ccc(Cl)cc12] | 115 | 78 | 7.4 | 15.1 |
| JKT.778A | | | | |
| 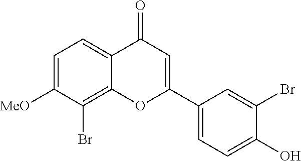 [SMILES: COc1ccc2c(=O)cc(-c3ccc(O)c(Br)c3)oc2c1Br] | 9 | 25 | >10 | <0.01 |
| JKT.780A | | | | |
| 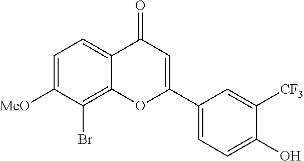 [SMILES: COc1ccc2c(=O)cc(-c3ccc(O)c(C(F)(F)F)c3)oc2c1Br] | 2475 | 1712 | >10 | <0.01 |
| JKT.786A | | | | |
| 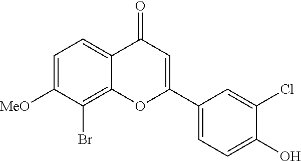 [SMILES: COc1ccc2c(=O)cc(-c3ccc(O)c(Cl)c3)oc2c1Br] | 24 | 16 | 7.7 | <0.01 |
| JKT.787A | | | | |

Given the success of the 2-pyridyl moiety, it was incorporated in analogues of JKT.464A. In another series, the extent to which the observations with JKT.706A generalize was evaluated. Additionally, C8-monobromo structures and thiazole analogues were prepared and evaluated.

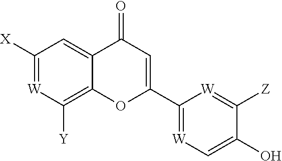

For W = CH or N,
Z = Br or Cl,
1) X = Y = Cl
2) X = Cl; Y = Br
3) X = Y = Br
4) X = H, Y = Br

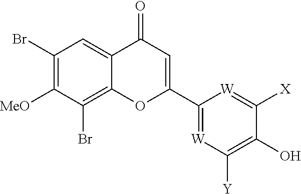

1) W = CH or N, X = Br, Y = H
2) W = CH, X = Y = Cl
3) W = CH, X = Y = F
4) W = CH or N, X = Cl, Y = H
5) W = CH, X = I, [symbol] Y = H
6) W = CH, X = CF3, Y = H
7) W = CH or N, X = alkynyl, Y = H
8) W = CH or N, X = cyclopropyl, Y = H

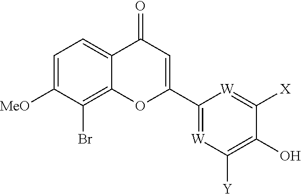

1) W = CH or N, X = Br, Y = H
2) W = CH, X = Y = Cl
3) W = CH, X = Y = F
4) W = CH or N, X = Cl, Y = H
5) W = CH, X = I, [symbol] Y = H
6) W = CH, X = CF3, Y = H
7) W = CH or N, X = alkynyl, Y = H
8) W = CH or N, X = cyclopropyl, Y = H

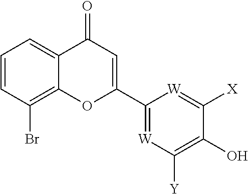

1) W = CH or N, X = Br, Y = H
2) W = CH, X = Y = Cl
3) W = CH, X = Y = F
4) W = CH or N, X = Cl, Y = H
5) W = CH, X = I, [symbol] Y = H
6) W = CH, X = CF3, Y = H
7) W = CH or N, X = alkynyl, Y = H
8) W = CH or N, X = cyclopropyl, Y = H

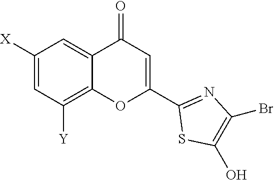

1) X = Y = Cl
2) X = Cl, Y = Br
3) X = Y = Br
4) X = H; Y = Br [symbol]
indicates data missing or illegible when filed

TABLE 4

| | | | | Aqueous |
|---|---|---|---|---|
| | CK2A1 RAD | CK2A2 RAD | IL6-Inhib. | Solubility |
| Structure | IC50 (nM) | IC50 (nM) | (μM) | (μM) |
| 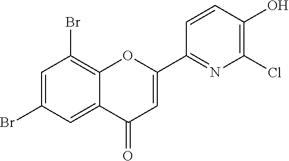 [SMILES: O=c1cc(-c2ccc(O)c(Cl)n2)oc2c(Br)cc(Br)cc12] | 24 | 21 | 4.4 | 154.9 |
| JKT.801A | | | | |
| 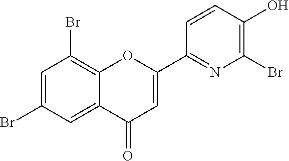 [SMILES: O=c1cc(-c2ccc(O)c(Br)n2)oc2c(Br)cc(Br)cc12] | 265 | 201 | 8 | 147.5 |
| JKT.844A | | | | |
| 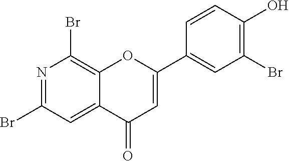 [SMILES: O=c1cc(-c2ccc(O)c(Br)c2)oc2c(Br)nc(Br)cc12] | 6 | 6 | N/A | N/A |
| JKT.909A | | | | |
| 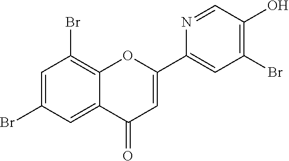 [SMILES: O=c1cc(-c2cc(Br)c(O)cn2)oc2c(Br)cc(Br)cc12] | 11 | 8 | N/A | N/A |
| JKT.923A | | | | |
| 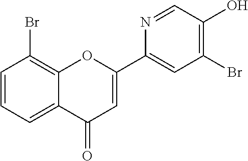 [SMILES: O=c1cc(-c2cc(Br)c(O)cn2)oc2c(Br)cccc12] | 53 | 55 | N/A | N/A |
| JKT.980A | | | | |

Additional compounds are shown in Table 5.

TABLE 5

| | | CK2A1 RAD | CK2A2 RAD |
|---|---|---|---|
| Compound ID | Structure | IC50 (nM) | IC50 (nM) |
| 1004A | 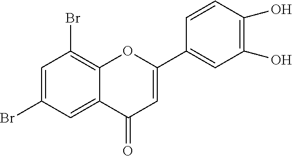 [SMILES: O=c1cc(-c2ccc(O)c(O)c2)oc2c(Br)cc(Br)cc12] | 845 | 803 |
| 1041A | 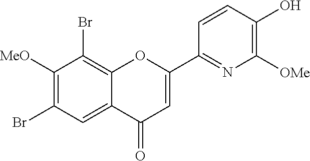 [SMILES: COc1nc(-c2cc(=O)c3cc(Br)c(OC)c(Br)c3o2)ccc1O] | 1889 | 1738 |
| 1058A | 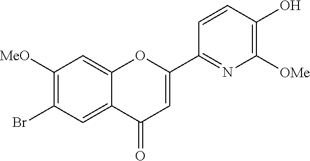 [SMILES: COc1cc2oc(-c3ccc(O)c(OC)n3)cc(=O)c2cc1Br] | 2726 | 1177 |
| 1059A | 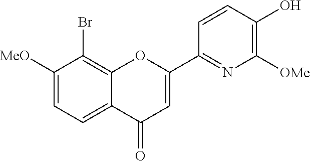 [SMILES: COc1ccc2c(=O)cc(-c3ccc(O)c(OC)n3)oc2c1Br] | 185 | 251 |
| 1040A | 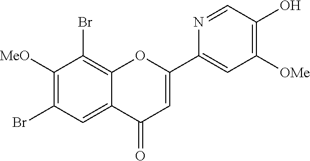 [SMILES: COc1cc(-c2cc(=O)c3cc(Br)c(OC)c(Br)c3o2)ncc1O] | 52 | 29 |
| 1063B | 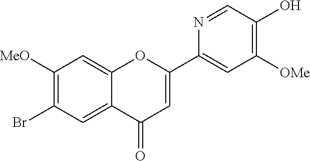 [SMILES: COc1cc(-c2cc(=O)c3cc(Br)c(OC)cc3o2)ncc1O] | 203 | 445 |
| 1064A | 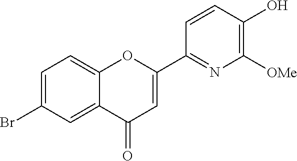 [SMILES: COc1nc(-c2cc(=O)c3cc(Br)ccc3o2)ccc1O] | 124 | 167 |
| 1065A | 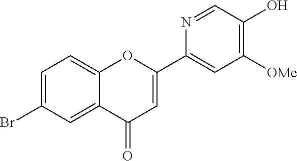 [SMILES: COc1cc(-c2cc(=O)c3cc(Br)ccc3o2)ncc1O] | 401 | 364 |
| 1132A | 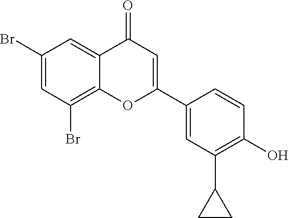 [SMILES: O=c1cc(-c2ccc(O)c(C3CC3)c2)oc2c(Br)cc(Br)cc12] | >5000 | >5000 |
| 1140A | 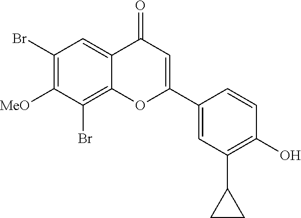 [SMILES: COc1c(Br)cc2c(=O)cc(-c3ccc(O)c(C4CC4)c3)oc2c1Br] | >5000 | >5000 |
| 1141A | 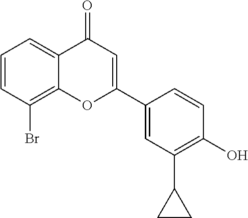 [SMILES: O=c1cc(-c2ccc(O)c(C3CC3)c2)oc2c(Br)cccc12] | >5000 | 4249 |
| 1142A | 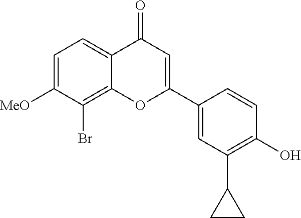 [SMILES: COc1ccc2c(=O)cc(-c3ccc(O)c(C4CC4)c3)oc2c1Br] | 2792 | 2226 |

Subsequently, quinolinones and quinolinone-like scaffolds were prepared to improve aqueous solubility.

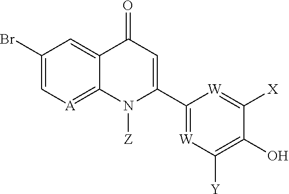

For A = N or A = C-Br: [symbol]
[i) For Z = H:
1) W = CH or N, X = Br, Y = H
2) W = CH, X = Y = Cl
3) W = CH, X = Y = F
4) W = CH or N, X = Cl, Y = H
5) W = CH, X = I, Y = H
6) W = CH, X = CF3, Y = H
7) W = CH or N, X = alkynyl, Y = H
8) W = CH or N, X = cyclopropyl, Y = H
9) W = CH or N, X = OMe, Y = H [symbol]
[i) For Z = cyclopropyl or Me; Any of
the patterns for W, X, and Y in 1-9 above]

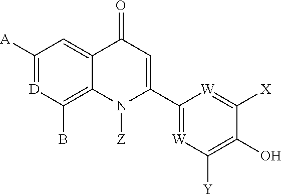

For A = B = Cl, A = B = Br, A = Cl & B = Br,
A = H & B = Br, and D = CH or N: [symbol]
[i) For Z = H:
1) W = CH or N, X = Br, Y = H
2) W = CH, X = Y = Cl
3) W = CH, X = Y = F
4) W = CH or N, X = Cl, Y = H
5) W = CH, X = I, Y = H
6) W = CH, X = CF3, Y = H
7) W = CH or N, X = alkynyl, Y = H
8) W = CH or N, X = cyclopropyl, Y = H
9) W = CH or N, X = OMe, Y = H [symbol]
[i) For Z = cyclopropyl or Me; Any of
the patterns for W, X, and Y
in 1-9 above]

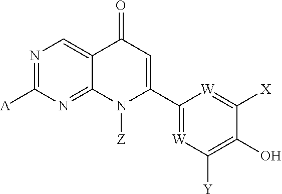

For A = H or OMe: [symbol]
[i) For Z = H:
1) W = CH or N, X = Br, Y = H
2) W = CH, X = Y = Cl
3) W = CH, X = Y = F
4) W = CH or N, X = Cl, Y = H
5) W = CH, X = I, Y = H
6) W = CH, X = CF3, Y = H
7) W = CH or N, X = alkynyl, Y = H
8) W = CH or N, X = cyclopropyl, Y = H
9) W = CH or N, X = OMe, Y = H [symbol]
[i) For Z = cyclopropyl or Me; Any of
the patterns for W, X, and Y
in 1-9 above] [symbol]
indicates data missing or illegible when filed

TABLE 6

| | | | | Aqueous |
|---|---|---|---|---|
| | CK2A1 RAD | CK2A2 RAD | IL6-Inhib. | Solubility |
| Structure | IC50 (nM) | IC50 (nM) | (μM) | (μM) |
| 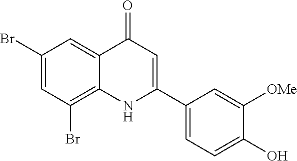 [SMILES: COc1cc(-c2cc(=O)c3cc(Br)cc(Br)c3[nH]2)ccc1O] | 22 | 15 | 3.9 | 10 |
| JKT.736A | | | | |
| 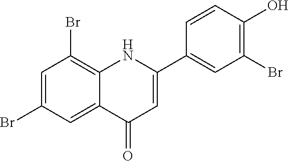 [SMILES: O=c1cc(-c2ccc(O)c(Br)c2)[nH]c2c(Br)cc(Br)cc12] | 128 | 123 | N/A | N/A |
| JKT.944A | | | | |

TABLE 7

| | | CK2A1 RAD | CK2A2 RAD |
|---|---|---|---|
| Compound ID | Structure | IC50 (nM) | IC50 (nM) |
| 1066A | 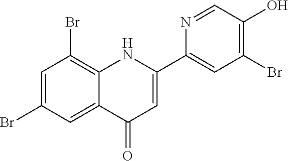 [SMILES: O=c1cc(-c2cc(Br)c(O)cn2)[nH]c2c(Br)cc(Br)cc12] | 2700 | 1290 |
| 1077A | 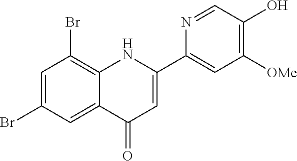 [SMILES: COc1cc(-c2cc(=O)c3cc(Br)cc(Br)c3[nH]2)ncc1O] | >5000 | >5000 |
| 1095B | 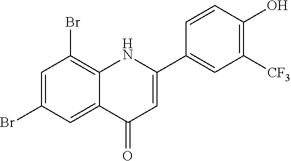 [SMILES: O=c1cc(-c2ccc(O)c(C(F)(F)F)c2)[nH]c2c(Br)cc(Br)cc12] | 488 | 699 |
| 1103A | 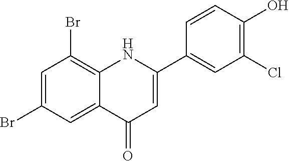 [SMILES: O=c1cc(-c2ccc(O)c(Cl)c2)[nH]c2c(Br)cc(Br)cc12] | 141 | 71 |

Quinazolinones and two classes of quinazolinone derivatives were evaluated. The cores were extended to also include more pyridyl moieties.

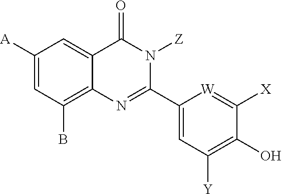

For A = B = Cl, A = B = Br, A = Cl & B = Br;
A = H & B = Cl, A = Cl & B = H,
A = Br & B = H, or A = H & B = Br: [symbol]
[i) For Z = H,
1) W = CH, X = CF3, Y = H
2) W = CH or N, X = Cl, Y = H
3) W = CH or N, X = OMe, Y = H
4) W = CH or N, X = Br, Y = H
5) W = CH, X = Y = Cl
6) W = CH, X = Y = F
7) W = CH or N, X = alkynyl, Y = H
8) W = CH or N, X = cyclopropyl, Y = H
9) W = CH or N, X = OMe, Y = H [symbol]
[i) For Z = cyclopropyl or Me;
Any of the patterns for
W, X, and Y in 1-9 above]

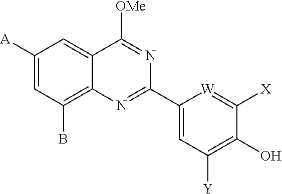

For A = B = Cl, A = B = Br, A = Cl & B = Br,
A = H & B = Cl, A = Cl & B = H,
A = Br & B = H, or A = H & B = Br:
1) W = CH, X = CF3, Y = H
2) W = CH or N, X = Cl, Y = H
3) W = CH or N, X = OMe, Y = H
4) W = CH or N, X = Br, Y = H
5) W = CH, X = Y = Cl
6) W = CH, X = Y = F
7) W = CH or N, X = alkynyl, Y = H
8) W = CH or N, X = cyclopropyl, Y = H
9) W = CH or N, X = OMe, Y = H

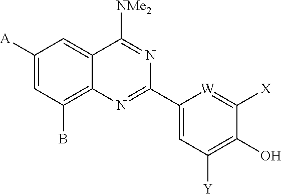

For A = B = Cl, A = B = Br, A = Cl & B = Br,
A = H & B = Cl, A = Cl & B = H,
A = Br & B = H, or A = H & B = Br:
1) W = CH, X = CF3, Y = H
2) W = CH or N, X = Cl, Y = H
3) W = CH or N, X = OMe, Y = H
4) W = CH or N, X = Br, Y = H
5) W = CH, X = Y = Cl
6) W = CH, X = Y = F
7) W = CH or N, X = alkynyl, Y = H
8) W = CH or N, X = cyclopropyl, Y = H
9) W = CH or N, X = OMe, Y = H [symbol]
indicates data missing or illegible when filed

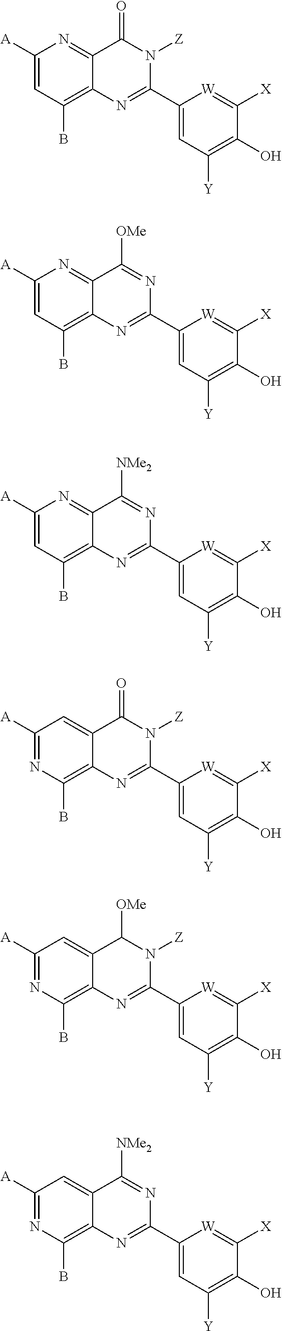

TABLE 8

| | | | | Aqueous |
|---|---|---|---|---|
| | CK2A1 RAD | CK2A2 RAD | IL6-Inhib. | Solubility |
| Structure | IC50 (nM) | IC50 (nM) | (μM) | (μM) |
| 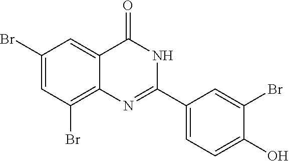 [SMILES: O=c1[nH]c(-c2ccc(O)c(Br)c2)nc2c(Br)cc(Br)cc12] | 24 | 42 | >10 | >200 |
| JKT.734C | | | | |
| 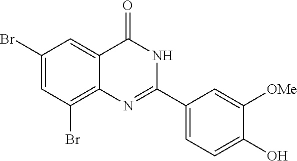 [SMILES: COc1cc(-c2nc3c(Br)cc(Br)cc3c(=O)[nH]2)ccc1O] | 863 | 2845 | >10 | N/A |
| JKT.737B | | | | |
| 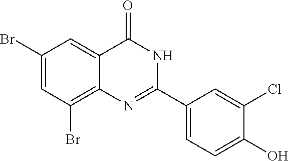 [SMILES: O=c1[nH]c(-c2ccc(O)c(Cl)c2)nc2c(Br)cc(Br)cc12] | 23 | 72 | 11 | <0.01 |
| JKT.772A | | | | |
| 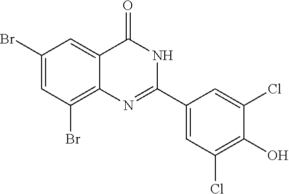 [SMILES: O=c1[nH]c(-c2cc(Cl)c(O)c(Cl)c2)nc2c(Br)cc(Br)cc12] | 243 | 276 | >10 | 24.1 |
| JKT.773B | | | | |
| 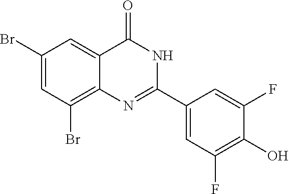 [SMILES: O=c1[nH]c(-c2cc(F)c(O)c(F)c2)nc2c(Br)cc(Br)cc12] | 818 | 1042 | >10 | 151.5 |
| JKT.774A | | | | |
| 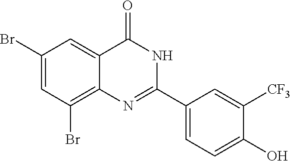 [SMILES: O=c1[nH]c(-c2ccc(O)c(C(F)(F)F)c2)nc2c(Br)cc(Br)cc12] | 370 | 2481 | >10 | 17.9 |
| JKT.775A | | | | |
| 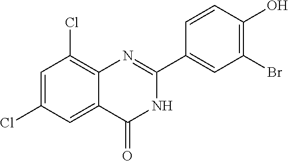 [SMILES: O=c1[nH]c(-c2ccc(O)c(Br)c2)nc2c(Cl)cc(Cl)cc12] | 2887 | 2827 | >10 | 4.2 |
| JKT.805A | | | | |
| 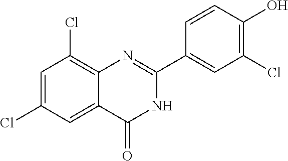 [SMILES: O=c1[nH]c(-c2ccc(O)c(Cl)c2)nc2c(Cl)cc(Cl)cc12] | >5000 | >5000 | >10 | 17 |
| JKT.806A | | | | |
| 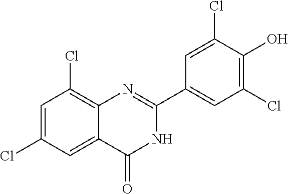 [SMILES: O=c1[nH]c(-c2cc(Cl)c(O)c(Cl)c2)nc2c(Cl)cc(Cl)cc12] | 910 | >5000 | >10 | 125.7 |
| JKT.807A | | | | |
| 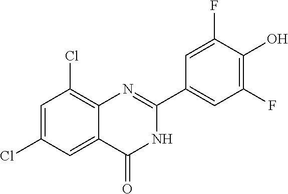 [SMILES: O=c1[nH]c(-c2cc(F)c(O)c(F)c2)nc2c(Cl)cc(Cl)cc12] | 2984 | 1624 | >10 | 188.7 |
| JKT.808A | | | | |
| 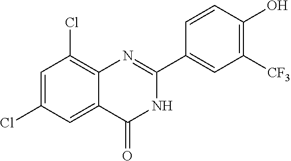 [SMILES: O=c1[nH]c(-c2ccc(O)c(C(F)(F)F)c2)nc2c(Cl)cc(Cl)cc12] | 82 | >5000 | >10 | <0.01 |
| JKT.809A | | | | |
| 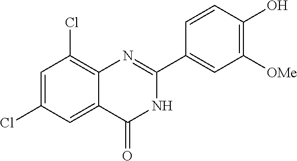 [SMILES: COc1cc(-c2nc3c(Cl)cc(Cl)cc3c(=O)[nH]2)ccc1O] | >5000 | >5000 | >10 | <0.01 |
| JKT.810A | | | | |
| 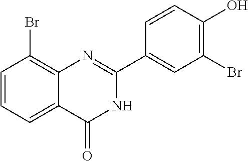 [SMILES: O=c1[nH]c(-c2ccc(O)c(Br)c2)nc2c(Br)cccc12] | 281 | 860 | >10 | 22.4 |
| JKT.827A | | | | |
| 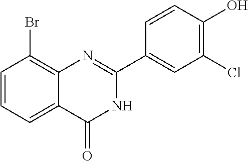 [SMILES: O=c1[nH]c(-c2ccc(O)c(Cl)c2)nc2c(Br)cccc12] | 652 | 1412 | >10 | 58.9 |
| JKT.828A | | | | |
| 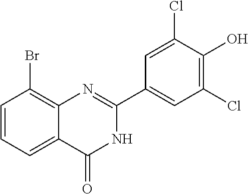 [SMILES: O=c1[nH]c(-c2cc(Cl)c(O)c(Cl)c2)nc2c(Br)cccc12] | 736 | 659 | >10 | 193.3 |
| JKT.829A | | | | |
| 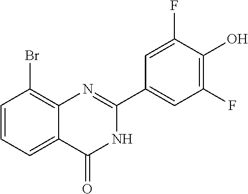 [SMILES: O=c1[nH]c(-c2cc(F)c(O)c(F)c2)nc2c(Br)cccc12] | 866 | 1209 | >10 | 171.1 |
| JKT.830A | | | | |
| 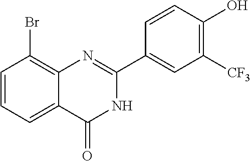 [SMILES: O=c1[nH]c(-c2ccc(O)c(C(F)(F)F)c2)nc2c(Br)cccc12] | >5000 | >5000 | >10 | 138.9 |
| JKT.831A | | | | |
| 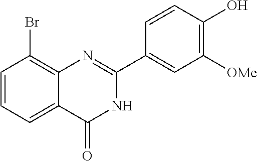 [SMILES: COc1cc(-c2nc3c(Br)cccc3c(=O)[nH]2)ccc1O] | 3945 | >5000 | 10.5 | 61.0 |
| JKT.832A | | | | |
| 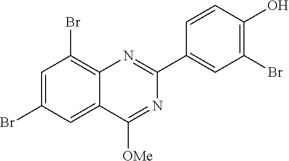 [SMILES: COc1nc(-c2ccc(O)c(Br)c2)nc2c(Br)cc(Br)cc12] | 1007 | 1130 | >10 | <0.01 |
| JKT.842A | | | | |
| 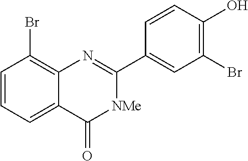 [SMILES: Cn1c(-c2ccc(O)c(Br)c2)nc2c(Br)cccc2c1=O] | 4586 | >5000 | N/A | N/A |
| JKT.866A | | | | |
| 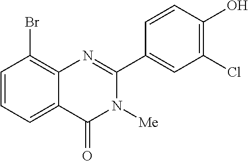 [SMILES: Cn1c(-c2ccc(O)c(Cl)c2)nc2c(Br)cccc2c1=O] | >5000 | >5000 | N/A | N/A |
| JKT.870A | | | | |
| 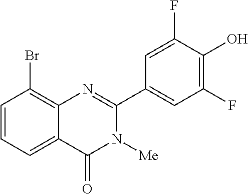 [SMILES: Cn1c(-c2cc(F)c(O)c(F)c2)nc2c(Br)cccc2c1=O] | 566 | 649 | N/A | N/A |
| JKT.873A | | | | |
| 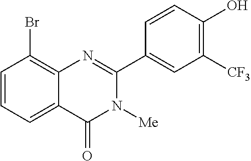 [SMILES: Cn1c(-c2ccc(O)c(C(F)(F)F)c2)nc2c(Br)cccc2c1=O] | >5000 | >5000 | N/A | N/A |
| JKT.875A | | | | |
| 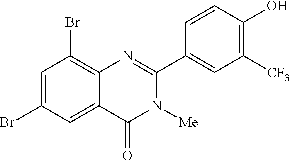 [SMILES: Cn1c(-c2ccc(O)c(C(F)(F)F)c2)nc2c(Br)cc(Br)cc2c1=O] | >5000 | >5000 | N/A | N/A |
| JKT.874D | | | | |
| 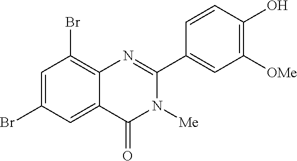 [SMILES: COc1cc(-c2nc3c(Br)cc(Br)cc3c(=O)n2C)ccc1O] | >5000 | >5000 | N/A | N/A |
| JKT.876A | | | | |
| 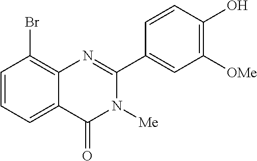 [SMILES: COc1cc(-c2nc3c(Br)cccc3c(=O)n2C)ccc1O] | >5000 | >5000 | N/A | N/A |
| JKT.877A | | | | |
| 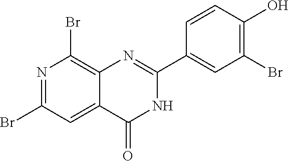 [SMILES: O=c1[nH]c(-c2ccc(O)c(Br)c2)nc2c(Br)nc(Br)cc12] | 95 | 242 | N/A | N/A |
| JKT.880B | | | | |
| 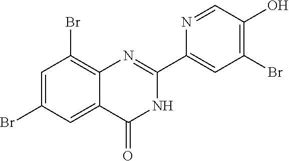 [SMILES: O=c1[nH]c(-c2cc(Br)c(O)cn2)nc2c(Br)cc(Br)cc12] | 107 | 99 | N/A | N/A |
| JKT.932F | | | | |
| 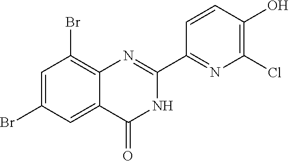 [SMILES: O=c1[nH]c(-c2ccc(O)c(Cl)n2)nc2c(Br)cc(Br)cc12] | 132 | 105 | N/A | N/A |
| JKT.956A | | | | |
| 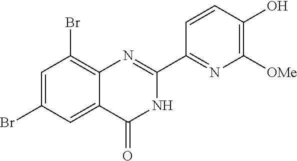 [SMILES: COc1nc(-c2nc3c(Br)cc(Br)cc3c(=O)[nH]2)ccc1O] | 27 | 33 | N/A | N/A |
| JKT.968C | | | | |
| 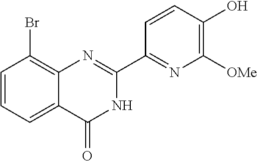 [SMILES: COc1nc(-c2nc3c(Br)cccc3c(=O)[nH]2)ccc1O] | 26 | 33 | N/A | N/A |
| JKT.975A | | | | |
| 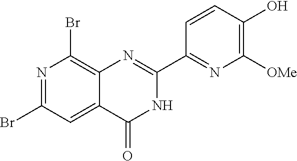 [SMILES: COc1nc(-c2nc3c(Br)nc(Br)cc3c(=O)[nH]2)ccc1O] | 234 | 495 | >10 | N/A |
| JKT.976B | | | | |
| 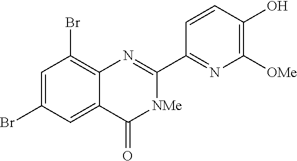 [SMILES: COc1nc(-c2nc3c(Br)cc(Br)cc3c(=O)n2C)ccc1O] | 677 | 900 | N/A | N/A |
| JKT.983A | | | | |

TABLE 9

| | | CK2A1 RAD | CK2A2 RAD |
|---|---|---|---|
| Compound ID | Structure | IC50 (nM) | IC50 (nM) |
| 963A | 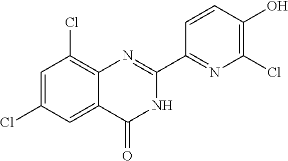 [SMILES: O=c1[nH]c(-c2ccc(O)c(Cl)n2)nc2c(Cl)cc(Cl)cc12] | 176 | 62 |
| 964A | 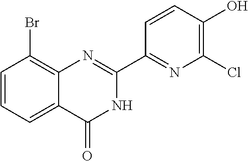 [SMILES: O=c1[nH]c(-c2ccc(O)c(Cl)n2)nc2c(Br)cccc12] | 155 | 137 |
| 1005A | 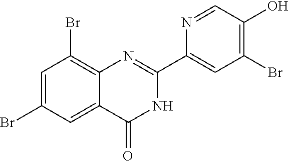 [SMILES: O=c1[nH]c(-c2cc(Br)c(O)cn2)nc2c(Br)cc(Br)cc12] | 94 | 53 |
| 1006A | 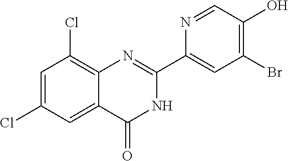 [SMILES: O=c1[nH]c(-c2cc(Br)c(O)cn2)nc2c(Cl)cc(Cl)cc12] | 182 | 85 |
| 1007A | 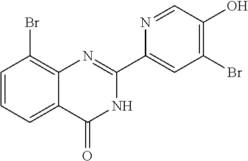 [SMILES: O=c1[nH]c(-c2cc(Br)c(O)cn2)nc2c(Br)cccc12] | 380 | 327 |

Next, a variety of aurones was synthesized and tested for CK2 inhibition and anti-inflammatory activity in cells. Subsets of these compounds have high potency at the biochemical and cellular levels and improved physicochemical properties, such as excellent solubility profiles (>100 μM; FIG. 12). For example, compound JKT.636A exhibited a nine-fold window of selectivity for CK2A2 over CK2A1, and JKT.634A displayed potent inhibition of both.

TABLE 10

| | CK2A1 IC50 | CK2A2 | Solubility | IL6-Inhib. |
|---|---|---|---|---|
| Name and Structure | (nM) | IC50 (nM) | (μM) | (μM) |
| 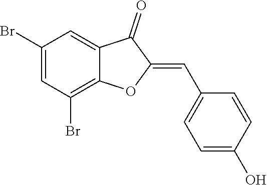 [SMILES: O=C1/C(=C/c2ccc(O)cc2)Oc2c(Br)cc(Br)cc21] | >5000 | >5000 | 5.6 | 7.9 |
| JKT.629A | | | | |
| 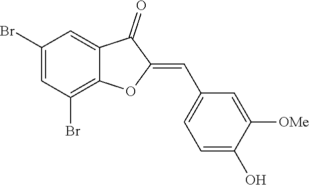 [SMILES: COc1cc(/C=C2\Oc3c(Br)cc(Br)cc3C2=O)ccc1O] | 211 | 274 | 3.0 | 9.8 |
| JKT.630A | | | | |
| 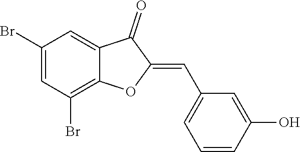 [SMILES: O=C1/C(=C/c2cccc(O)c2)Oc2c(Br)cc(Br)cc21] | >5000 | >5000 | 22.6 | >10 |
| JKT.631A | | | | |
| 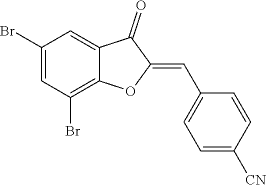 [SMILES: N#Cc1ccc(/C=C2\Oc3c(Br)cc(Br)cc3C2=O)cc1] | >5000 | >5000 | >200 | 10 |
| JKT.632A | | | | |
| 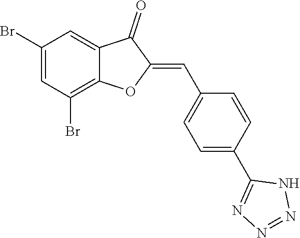 [SMILES: O=C1/C(=C/c2ccc(-c3nnn[nH]3)cc2)Oc2c(Br)cc(Br)cc21] | >5000 | >5000 | 102.2 | >10 |
| JKT.633A | | | | |
| 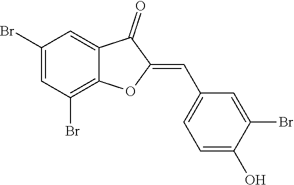 [SMILES: O=C1/C(=C/c2ccc(O)c(Br)c2)Oc2c(Br)cc(Br)cc21] | 22 | 46 | 25.5 | 2 |
| JKT.634A | | | | |
| 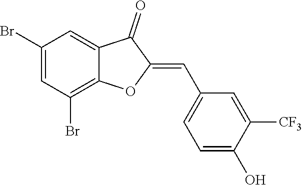 [SMILES: O=C1/C(=C/c2ccc(O)c(C(F)(F)F)c2)Oc2c(Br)cc(Br)cc21] | 531 | 213 | 22 | 1.8 |
| JKT.635A | | | | |
| 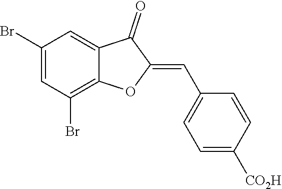 [SMILES: O=C(O)c1ccc(/C=C2\Oc3c(Br)cc(Br)cc3C2=O)cc1] | 537 | 62 | >200 | >10 |
| JKT.636A | | | | |
| 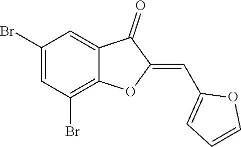 [SMILES: O=C1/C(=C/c2ccco2)Oc2c(Br)cc(Br)cc21] | >5000 | >5000 | 52.4 | >10 |
| JKT.637A | | | | |
| 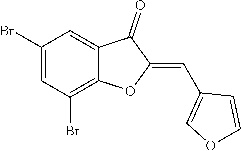 [SMILES: O=C1/C(=C/c2ccoc2)Oc2c(Br)cc(Br)cc21] | >5000 | >5000 | 30.2 | >10 |
| JKT.638A | | | | |

Additional compounds include aurones with a 2-pyridyl element, as well as C7-methoxylated aurones.

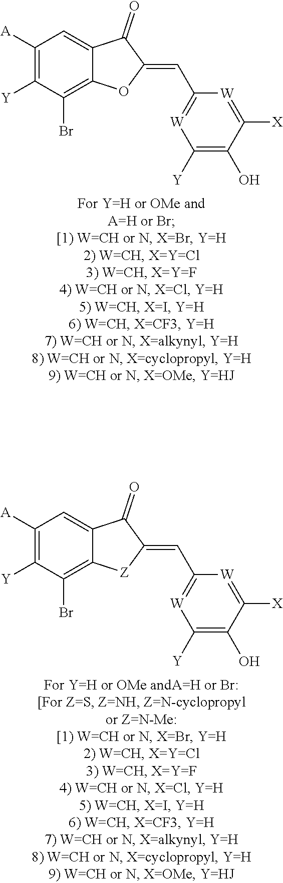

TABLE 11

| | | | | Aqueous |
|---|---|---|---|---|
| | CK2A1 RAD | CK2A2 RAD | IL6-Inhib. | Solubility |
| Structure | IC50 (nM) | IC50 (nM) | (μM) | (μM) |
| 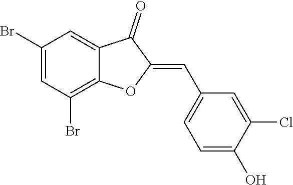 [SMILES: O=C1/C(=C/c2ccc(O)c(Cl)c2)Oc2c(Br)cc(Br)cc21] | 44 | 26 | 3.4 | 7.8 |
| JKT.726A | | | | |
| 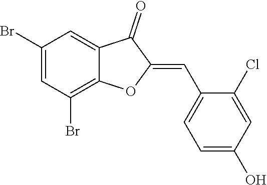 [SMILES: O=C1/C(=C/c2ccc(O)cc2Cl)Oc2c(Br)cc(Br)cc21] | >5000 | >5000 | 4.7 | 73.7 |
| JKT.727A | | | | |
| 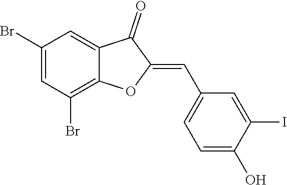 [SMILES: O=C1/C(=C/c2ccc(O)c(I)c2)Oc2c(Br)cc(Br)cc21] | 81 | 70 | 1.7 | 17.7 |
| JKT.728A | | | | |
| 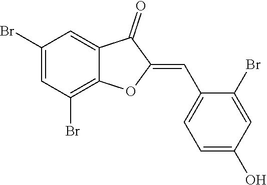 [SMILES: O=C1/C(=C/c2ccc(O)cc2Br)Oc2c(Br)cc(Br)cc21] | >5000 | >5000 | 3.0 | 2.7 |
| JKT.758A | | | | |
| 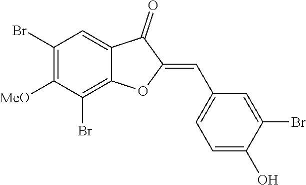 [SMILES: COc1c(Br)cc2c(c1Br)O/C(=C\c1ccc(O)c(Br)c1)C2=O] | 57 | 44 | 2.3 | 3.6 |
| JKT.759A | | | | |
| 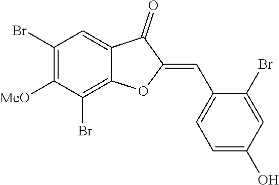 [SMILES: COc1c(Br)cc2c(c1Br)O/C(=C\c1ccc(O)cc1Br)C2=O] | >5000 | >5000 | 9.4 | 183.5 |
| JKT.760A | | | | |
| 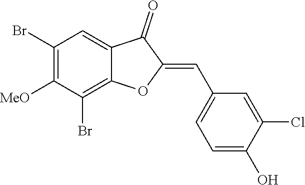 [SMILES: COc1c(Br)cc2c(c1Br)O/C(=C\c1ccc(O)c(Cl)c1)C2=O] | 67 | 48 | 2.5 | 119.8 |
| JKT.761A | | | | |
| 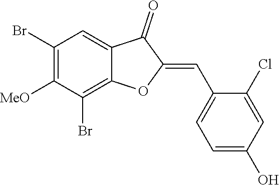 [SMILES: COc1c(Br)cc2c(c1Br)O/C(=C\c1ccc(O)cc1Cl)C2=O] | >5000 | >5000 | 4.4 | 58.6 |
| JKT.762A | | | | |
| 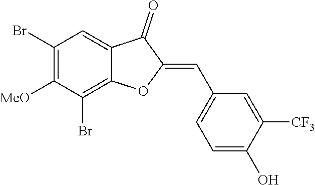 [SMILES: COc1c(Br)cc2c(c1Br)O/C(=C\c1ccc(O)c(C(F)(F)F)c1)C2=O] | 598 | 627 | 2.7 | 7.9 |
| JKT.763A | | | | |
| 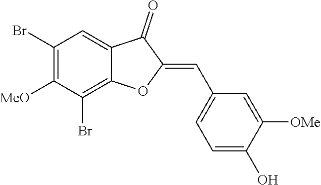 [SMILES: COc1cc(/C=C2\Oc3c(cc(Br)c(OC)c3Br)C2=O)ccc1O] | 521 | 383 | 12 | <0.01 |
| JKT.764A | | | | |
| 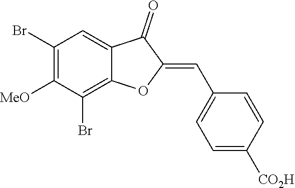 [SMILES: COc1c(Br)cc2c(c1Br)O/C(=C\c1ccc(C(=O)O)cc1)C2=O] | 2455 | 199 | 15 | >200 |
| JKT.765A | | | | |
| 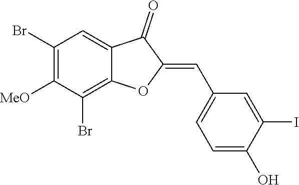 [SMILES: COc1c(Br)cc2c(c1Br)O/C(=C\c1ccc(O)c(I)c1)C2=O] | 135 | 193 | 1.1 | <0.01 |
| JKT.813A | | | | |
| 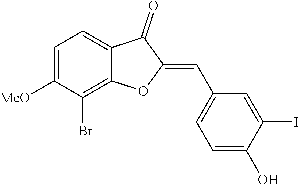 [SMILES: COc1ccc2c(c1Br)O/C(=C\c1ccc(O)c(I)c1)C2=O] | 131 | 123 | 4.3 | <0.01 |
| JKT.814A | | | | |
| 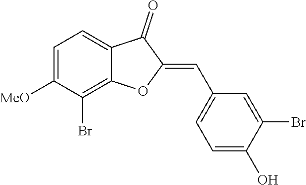 [SMILES: COc1ccc2c(c1Br)O/C(=C\c1ccc(O)c(Br)c1)C2=O] | 47 | 26 | 4.6 | <0.01 |
| JKT.815A | | | | |
| 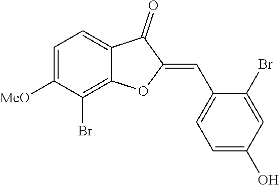 [SMILES: COc1ccc2c(c1Br)O/C(=C\c1ccc(O)cc1Br)C2=O] | 119 | 1523 | >10 | <0.01 |
| JKT.816A | | | | |
| 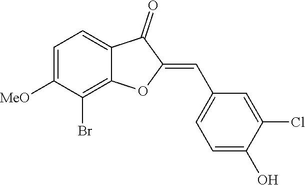 [SMILES: COc1ccc2c(c1Br)O/C(=C\c1ccc(O)c(Cl)c1)C2=O] | 59 | 53 | 6.5 | <0.01 |
| JKT.817A | | | | |
| 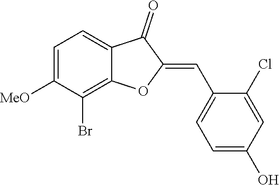 [SMILES: COc1ccc2c(c1Br)O/C(=C\c1ccc(O)cc1Cl)C2=O] | >5000 | >5000 | >10 | <0.01 |
| JKT.818A | | | | |
| 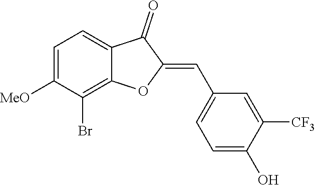 [SMILES: COc1ccc2c(c1Br)O/C(=C\c1ccc(O)c(C(F)(F)F)c1)C2=O] | 540 | 660 | 5.9 | 17 |
| JKT.819A | | | | |
| 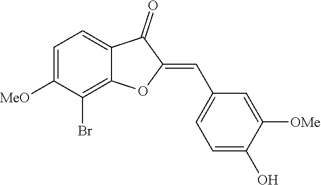 [SMILES: COc1cc(/C=C2\Oc3c(ccc(OC)c3Br)C2=O)ccc1O] | 7 | 6 | >10 | <0.01 |
| JKT.820A | | | | |
| 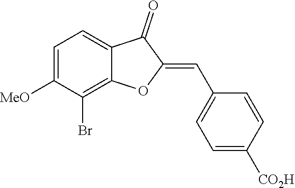 [SMILES: COc1ccc2c(c1Br)O/C(=C\c1ccc(C(=O)O)cc1)C2=O] | 1535 | 206 | >10 | 195.7 |
| JKT.821A | | | | |
| 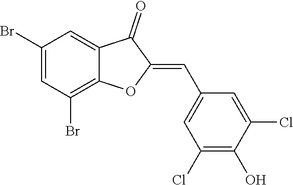 [SMILES: O=C1/C(=C/c2cc(Cl)c(O)c(Cl)c2)Oc2c(Br)cc(Br)cc21] | 2011 | 804 | 2 | <0.01 |
| JKT.822A | | | | |
| 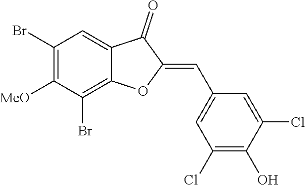 [SMILES: COc1c(Br)cc2c(c1Br)O/C(=C\c1cc(Cl)c(O)c(Cl)c1)C2=O] | >5000 | 1224 | 5.5 | 166.6 |
| JKT.823A | | | | |
| 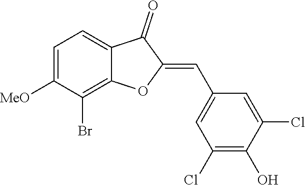 [SMILES: COc1ccc2c(c1Br)O/C(=C\c1cc(Cl)c(O)c(Cl)c1)C2=O] | 447 | 140 | 10.1 | >200 |
| JKT.824A | | | | |

Additional compounds are shown in Table 12.

TABLE 12

| | | CK2A1 RAD | CK2A2 RAD |
|---|---|---|---|
| Compound ID | Structure | IC50 (nM) | IC50 (nM) |
| 1131A | 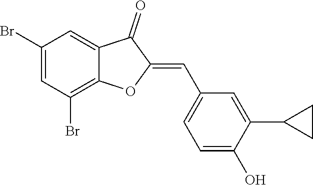 [SMILES: O=C1/C(=C/c2ccc(O)c(C3CC3)c2)Oc2c(Br)cc(Br)cc21] | >5000 | >5000 |
| 1134A | 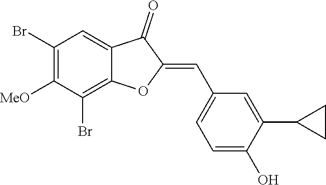 [SMILES: COc1c(Br)cc2c(c1Br)O/C(=C\c1ccc(O)c(C3CC3)c1)C2=O] | >5000 | >5000 |
| 1143A | 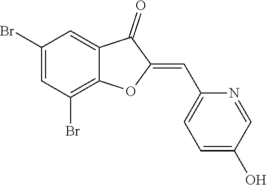 [SMILES: O=C1/C(=C/c2ccc(O)cn2)Oc2c(Br)cc(Br)cc21] | >5000 | >5000 |
| 1135B | 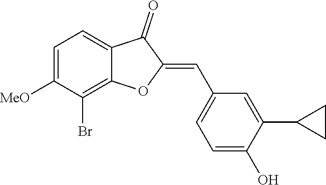 [SMILES: COc1ccc2c(c1Br)O/C(=C\c1ccc(O)c(C3CC3)c1)C2=O] | >5000 | >5000 |
| 1150A | 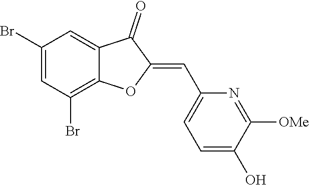 [SMILES: COc1nc(/C=C2\Oc3c(Br)cc(Br)cc3C2=O)ccc1O] | 178 | 109 |
| 1159A | 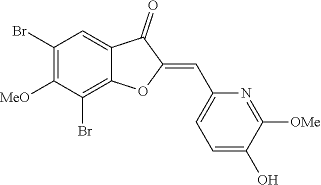 [SMILES: COc1nc(/C=C2\Oc3c(cc(Br)c(OC)c3Br)C2=O)ccc1O] | 1020 | 590 |
| 1163A | 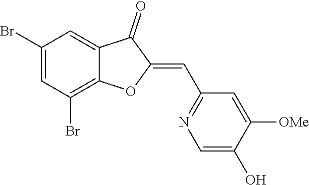 [SMILES: COc1cc(/C=C2\Oc3c(Br)cc(Br)cc3C2=O)ncc1O] | 45 | 403 |
| 1173A | 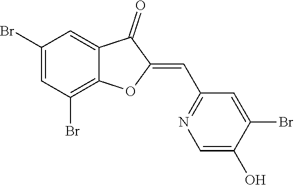 [SMILES: O=C1/C(=C/c2cc(Br)c(O)cn2)Oc2c(Br)cc(Br)cc21] | 60 | 30 |
| 1174A | 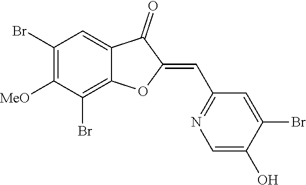 [SMILES: COc1c(Br)cc2c(c1Br)O/C(=C\c1cc(Br)c(O)cn1)C2=O] | 86 | 27 |
| 1188A | 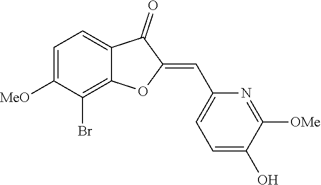 [SMILES: COc1ccc2c(c1Br)O/C(=C\c1ccc(O)c(OC)n1)C2=O] | 465 | 144 |
| 1189A | 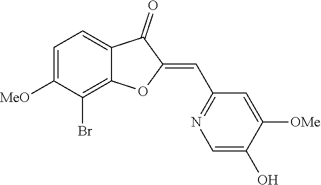 [SMILES: COc1cc(/C=C2\Oc3c(ccc(OC)c3Br)C2=O)ncc1O] | 117 | 149 |
| 1210A | 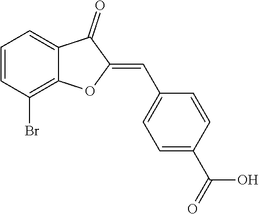 [SMILES: O=C(O)c1ccc(/C=C2\Oc3c(Br)cccc3C2=O)cc1] | 703 | 60 |
| 1212A | 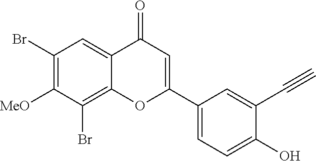 [SMILES: C#Cc1cc(-c2cc(=O)c3cc(Br)c(OC)c(Br)c3o2)ccc1O] | >5000 | 1260 |
| 1220A | 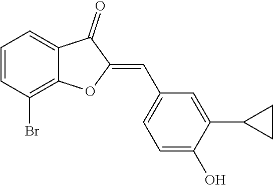 [SMILES: O=C1/C(=C/c2ccc(O)c(C3CC3)c2)Oc2c(Br)cccc21] | 4452 | >5000 |
| 1224A | 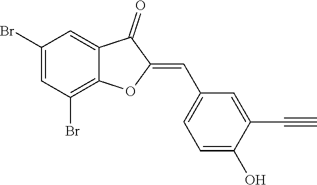 [SMILES: C#Cc1cc(/C=C2\Oc3c(Br)cc(Br)cc3C2=O)ccc1O] | 177 | 77 |
| 1229A | 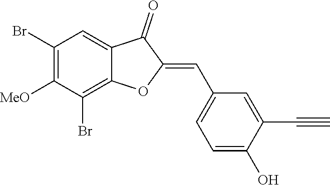 [SMILES: C#Cc1cc(/C=C2\Oc3c(cc(Br)c(OC)c3Br)C2=O)ccc1O] | 151 | 49 |
| 1164A | 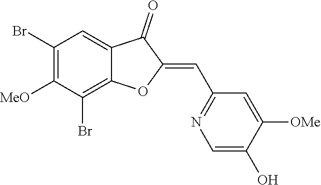 [SMILES: COc1cc(/C=C2\Oc3c(cc(Br)c(OC)c3Br)C2=O)ncc1O] | 120 | 331 |
| 1226A | 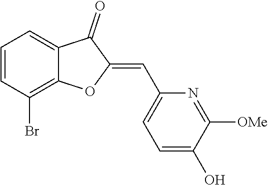 [SMILES: COc1nc(/C=C2\Oc3c(Br)cccc3C2=O)ccc1O] | 131 | 91 |
| 1231A | 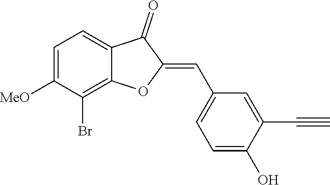 [SMILES: C#Cc1cc(/C=C2\Oc3c(ccc(OC)c3Br)C2=O)ccc1O] | 180 | 206 |
| 1190B | 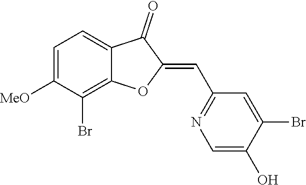 [SMILES: COc1ccc2c(c1Br)O/C(=C\c1cc(Br)c(O)cn1)C2=O] | 184 | 46 |
| 1237A | 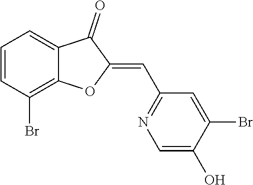 [SMILES: O=C1/C(=C/c2cc(Br)c(O)cn2)Oc2c(Br)cccc21] | 67 | 57 |
| 1236B | 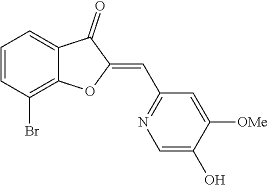 [SMILES: COc1cc(/C=C2\Oc3c(Br)cccc3C2=O)ncc1O] | 172 | 141 |

In view of the many possible embodiments to which the principles of the disclosed disclosure may be applied, it should be recognized that the illustrated embodiments are only examples of the disclosure and should not be taken as limiting the scope of the disclosure. Rather, the scope of the disclosure is defined by the following claims. We therefore claim as our invention all that comes within the scope and spirit of these claims.

1. A compound according to formula I or formula II, or a pharmaceutically acceptable salt, hydrate, stereoisomer, or tautomer thereof: 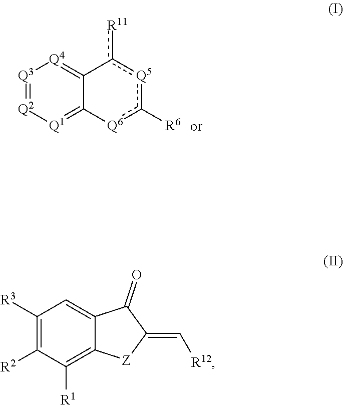
where each bond represented by “ [symbol] ” is a single or double bond as needed to satisfy valence requirements;
Q¹ is C—R¹ or N;
Q² is C—R² or N;
Q³ is C—R³ or N;
Q⁴ is C—R⁴ or N;
Q⁵ is C—R⁵, N, or N—R^{b};
Q⁶ is O, N, or N—R^{b};
Z is O, S, or N—R^{b};
R¹ is X or H;
R² is H or —OR^{a};
R³ is X or H;
R⁴ is H;
R⁵ is H;
R⁶ is 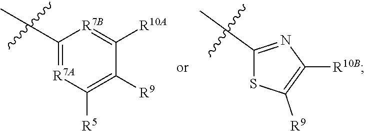
R^{7A} and R^{7B} independently are N or CH;
R⁸ is H or X;
R⁹ is hydroxy;
R^{10A} and R^{10B} independently are X, —OR^{a}, C₁-C₅ aliphatic, substituted C₁-C₅ aliphatic, or cycloaliphatic;
R¹¹ is O, —OR^{a}, or N(R^{a})₂;
R¹² is 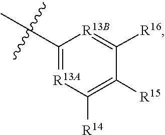
furan-2-yl, or furan-3-yl;
R^{13A} and R^{13B} independently are N or CH;
R¹⁴ is H, X, or —OR^{a};
R¹⁵ is —OR^{a}, H, cyano, tetrazolyl, or —CO₂R^{a};
R¹⁶ is H, X, —OR^{a}, substituted aliphatic, cycloaliphatic, or alkynyl;
each R^{a} independently is H or C₁-C₅ aliphatic;
each R^{b} independently is H, C₁-C₅ aliphatic, or cycloaliphatic; and
X is halo,
wherein, when the compound has a structure according to formula I, at least one of Q¹-Q⁴ is N, or Q⁵ is N or N—R^{b}, or Q⁶ is N or N—R^{b}, or R¹¹ is other than O.

2. The compound according to claim 1, wherein:
R¹ and R³ are X, and R² is H or methoxy; or
R¹ is X, R² is methoxy or H, and R³ is H; or
R¹ and R² are H, and R³ is X.

3. The compound according to claim 1, having a structure according to formula I, wherein:
Q⁵ is C—R⁵;
Q⁶ is O; and
R¹¹ is O.

4. The compound according to claim 1, having a structure according to formula I, wherein:
Q¹ is C—R¹ and R¹ is X;
Q² is N or CH;
Q³ is C—R³ and R³ is X; and
Q⁴ is C—R⁴.

5. The compound according to claim 1, wherein each X independently is Br or Cl.

6. The compound according to claim 1, having a structure according to formula I, wherein R⁶ is 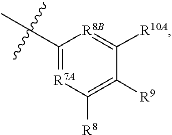
where:
(i) R⁹ is hydroxy; or
(ii) R^{10A} and R^{10B} independently are Br, Cl, F, I, —CF₃, alkynyl, cyclopropyl, or —OCH₃; or
(iii) both (i) and (ii).

7. The compound according to claim 1, having a structure according to formula I-A or I-B: 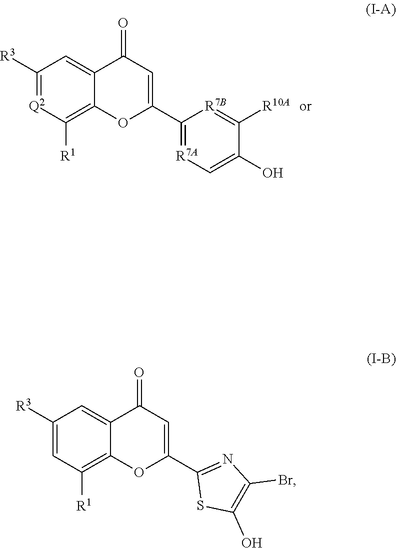
where R¹ is Br or Cl;
R³ is Br, Cl, or H;
Q², R^{7A}, and R^{7B} independently are N or CH; and
R^{10A} is Br or Cl.

8. The compound according to claim 1, having a structure according to formula I-C, I-D, or I-E: 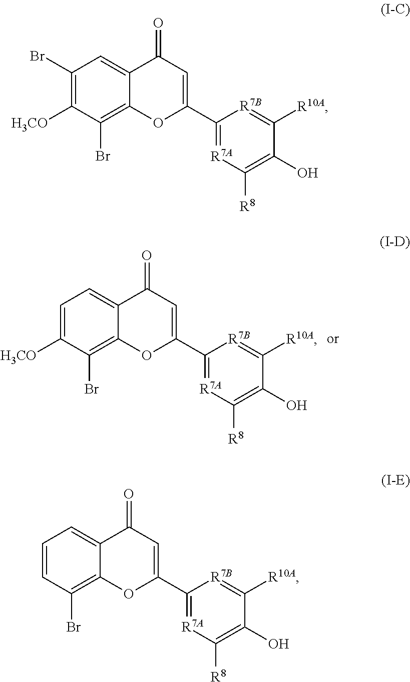
where R^{7A} and R^{7B} independently are N or CH;
R⁸ is H, Cl, or F; and
R^{10A} is Br, Cl, F, I, —CF₃, alkynyl, or cyclopropyl.

9. The compound according to claim 1, having a structure according to formula I-F: 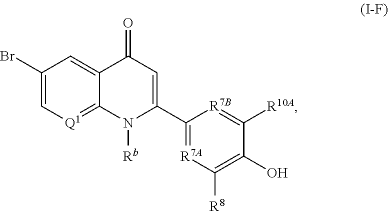
where Q¹ is N or C—Br;
R^{b} is H, —CH₃, or cyclopropyl;
R^{7A} and R^{7B} independently are N or CH;
R⁸ is H, Cl, or F; and
R^{10A} is Br, Cl, F, I, —CF₃, alkynyl, cyclopropyl, or —OCH₃.

10. The compound according to claim 1, having a structure according to formula I-G: 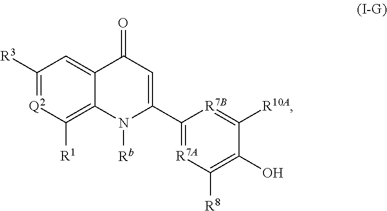
where Q², R^{7A}, and R^{7B} independently are N or CH;
R¹ is Cl or Br;
R³ is Cl, Br, or H;
R^{b} is H, —CH₃, or cyclopropyl;
R⁸ is H, Cl, or F; and
R^{10A} is Br, Cl, F, I, —CF₃, alkynyl, cyclopropyl, or —OCH₃.

11. The compound according to claim 1, having a structure according to formula I-H: 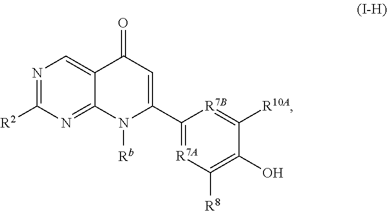
where R² is H or —OCH₃;
R^{b} is H, —CH₃, or cyclopropyl;
R^{7A} and R^{7B} independently are N or CH;
R⁸ is H, Cl, or F; and
R^{10A} is Br, Cl, F, I, —CF₃, alkynyl, cyclopropyl, or —OCH₃.

12. The compound according to claim 1, having a structure according to any one of formulas I-I to I-Q: 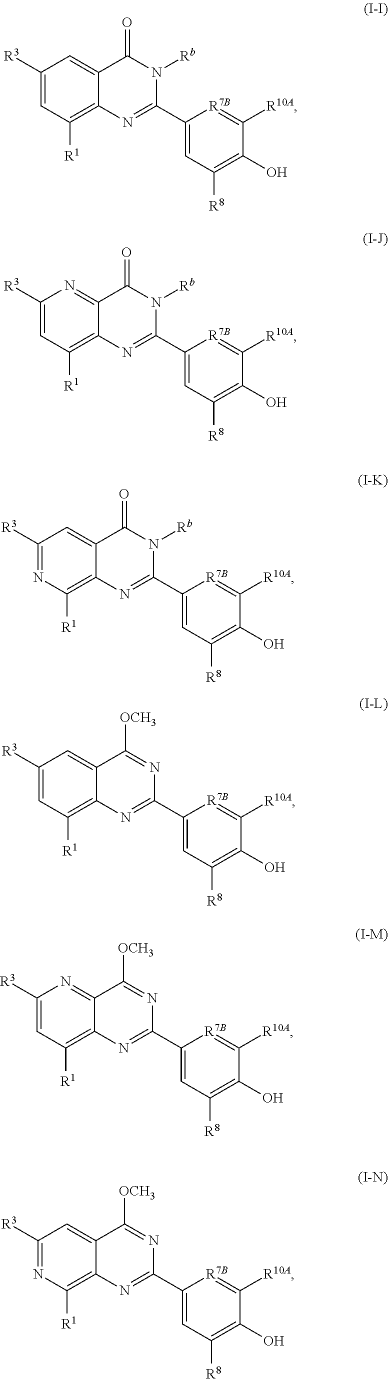 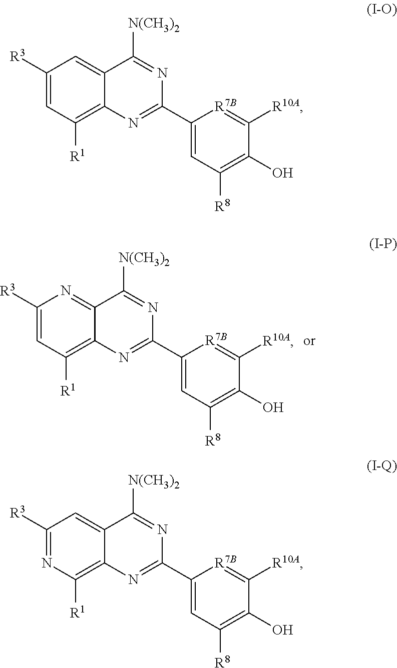
where R¹ and R³ independently are Cl, Br, or H;
R^{b} is H, —CH₃, or cyclopropyl;
R^{7B} is CH or N;
R⁸ is H, Cl, or F; and
R^{10A} is Br, Cl, F, I, —CF₃, alkynyl, cyclopropyl, or —OCH₃.

13. The compound according to claim 1, having a structure according to formula II-A 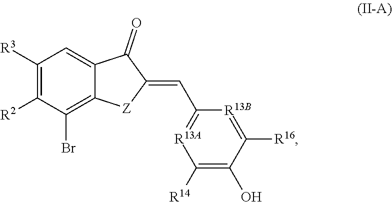
where Z is O, S, N(H), N—CH₃, or N-cyclopropyl;
R² is H or —OCH₃;
R³ is H or Br;
R^{13A} and R^{13B} independently are CH or N;
R¹⁴ is H, Cl, or F; and
R¹⁶ is Br, Cl, F, I, —CF₃, alkynyl, cyclopropyl, or —OCH₃.

14. (canceled)

15. The compound according to claim 1, wherein the compound is:

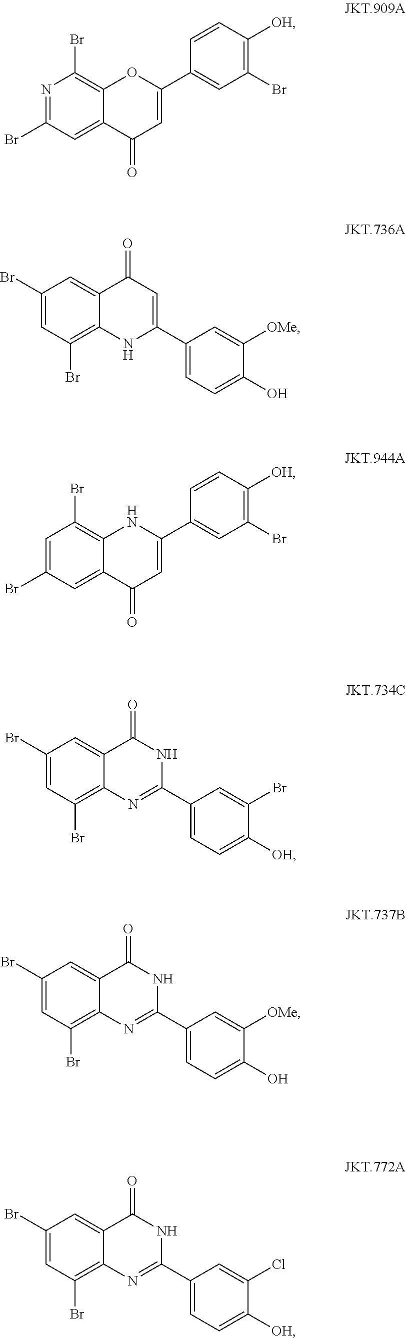 [SMILES: O=c1cc(-c2ccc(O)c(Br)c2)oc2c(Br)nc(Br)cc12 ; COc1cc(-c2cc(=O)c3cc(Br)cc(Br)c3[nH]2)ccc1O ; O=c1cc(-c2ccc(O)c(Br)c2)[nH]c2c(Br)cc(Br)cc12 ; O=c1[nH]c(-c2ccc(O)c(Br)c2)nc2c(Br)cc(Br)cc12 ; COc1cc(-c2nc3c(Br)cc(Br)cc3c(=O)[nH]2)ccc1O ; O=c1[nH]c(-c2ccc(O)c(Cl)c2)nc2c(Br)cc(Br)cc12]

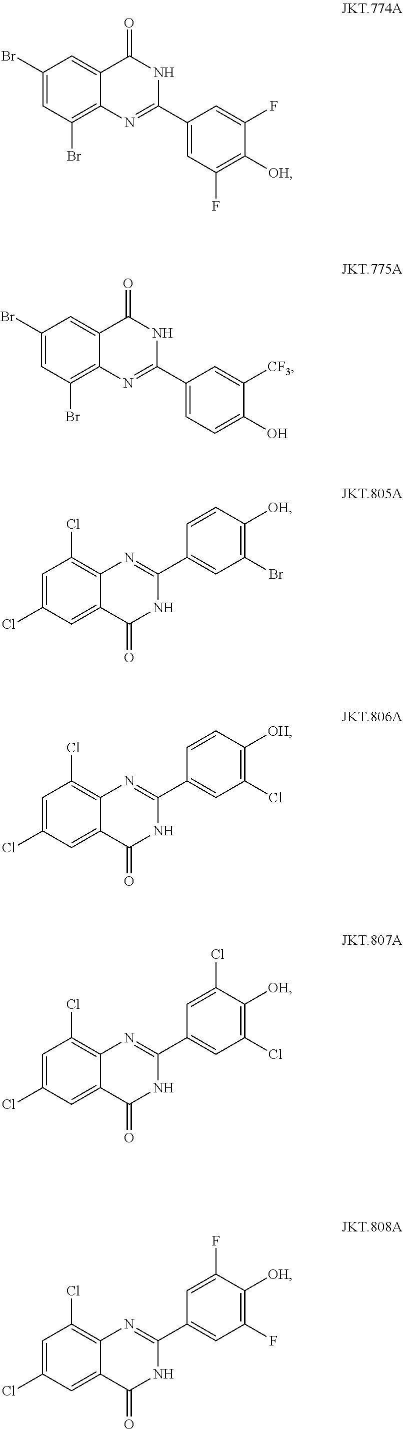 [SMILES: O=c1[nH]c(-c2cc(F)c(O)c(F)c2)nc2c(Br)cc(Br)cc12 ; O=c1[nH]c(-c2ccc(O)c(C(F)(F)F)c2)nc2c(Br)cc(Br)cc12 ; O=c1[nH]c(-c2ccc(O)c(Br)c2)nc2c(Cl)cc(Cl)cc12 ; O=c1[nH]c(-c2ccc(O)c(Cl)c2)nc2c(Cl)cc(Cl)cc12 ; O=c1[nH]c(-c2cc(Cl)c(O)c(Cl)c2)nc2c(Cl)cc(Cl)cc12 ; O=c1[nH]c(-c2cc(F)c(O)c(F)c2)nc2c(Cl)cc(Cl)cc12]

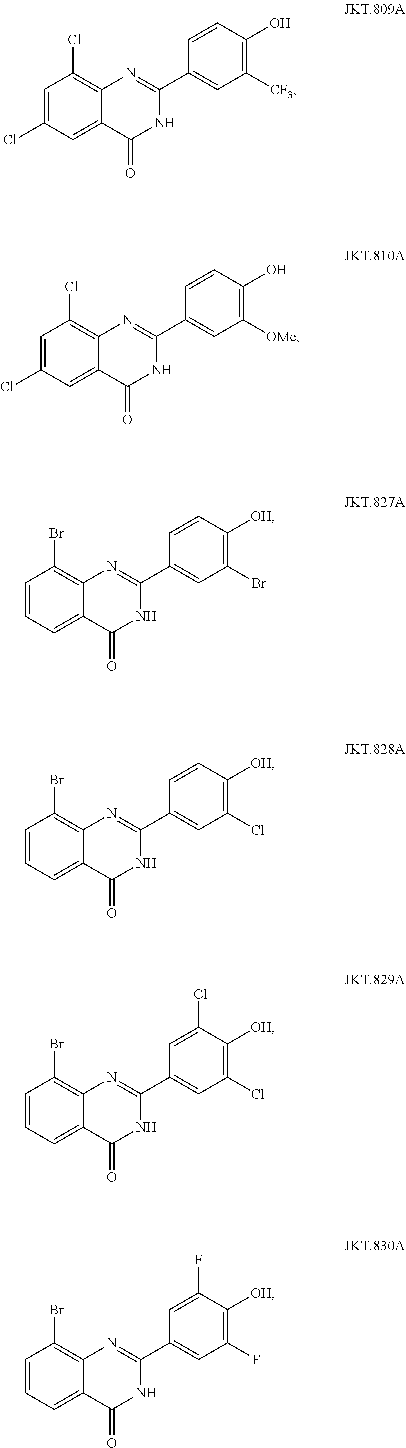 [SMILES: O=c1[nH]c(-c2ccc(O)c(C(F)(F)F)c2)nc2c(Cl)cc(Cl)cc12 ; COc1cc(-c2nc3c(Cl)cc(Cl)cc3c(=O)[nH]2)ccc1O ; O=c1[nH]c(-c2ccc(O)c(Br)c2)nc2c(Br)cccc12 ; O=c1[nH]c(-c2ccc(O)c(Cl)c2)nc2c(Br)cccc12 ; O=c1[nH]c(-c2cc(Cl)c(O)c(Cl)c2)nc2c(Br)cccc12 ; O=c1[nH]c(-c2cc(F)c(O)c(F)c2)nc2c(Br)cccc12]

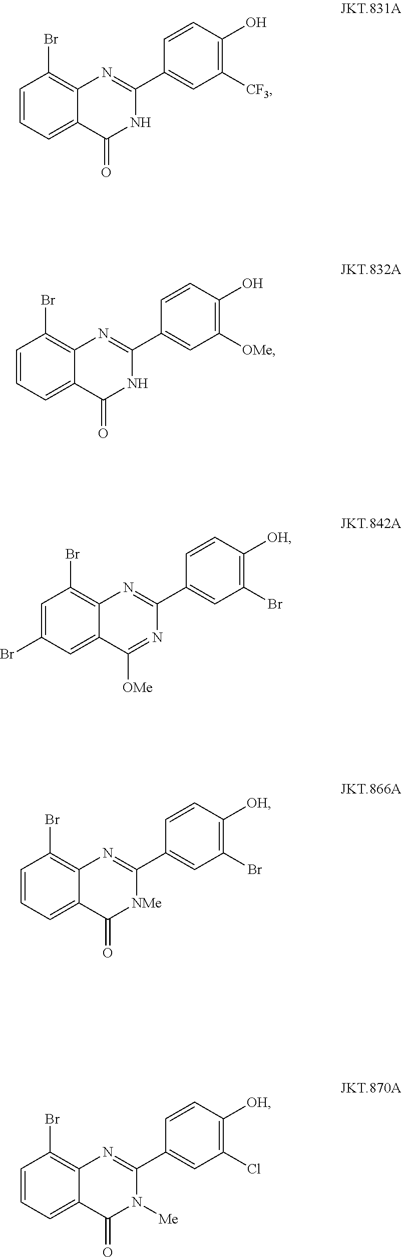 [SMILES: O=c1[nH]c(-c2ccc(O)c(C(F)(F)F)c2)nc2c(Br)cccc12 ; COc1cc(-c2nc3c(Br)cccc3c(=O)[nH]2)ccc1O ; COc1nc(-c2ccc(O)c(Br)c2)nc2c(Br)cc(Br)cc12 ; Cn1c(-c2ccc(O)c(Br)c2)nc2c(Br)cccc2c1=O ; Cn1c(-c2ccc(O)c(Cl)c2)nc2c(Br)cccc2c1=O]

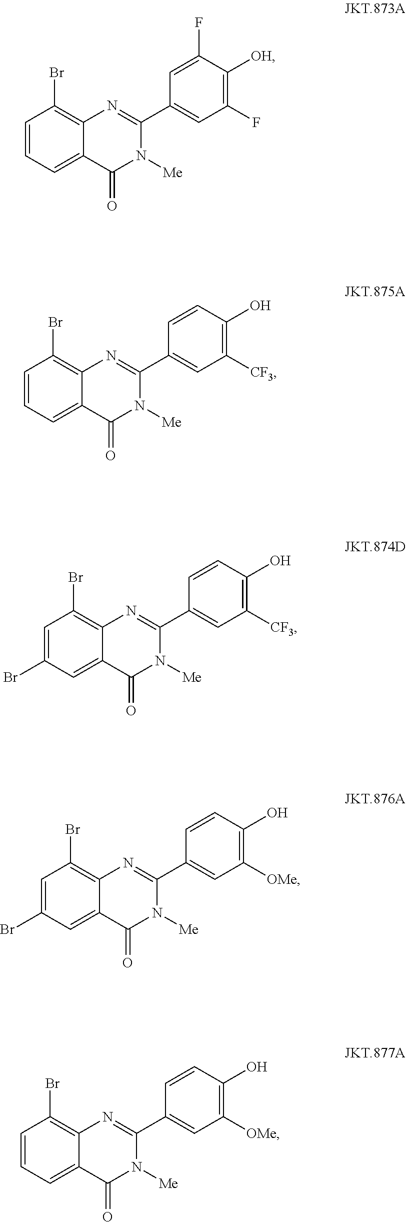 [SMILES: Cn1c(-c2cc(F)c(O)c(F)c2)nc2c(Br)cccc2c1=O ; Cn1c(-c2ccc(O)c(C(F)(F)F)c2)nc2c(Br)cccc2c1=O ; Cn1c(-c2ccc(O)c(C(F)(F)F)c2)nc2c(Br)cc(Br)cc2c1=O ; COc1cc(-c2nc3c(Br)cc(Br)cc3c(=O)n2C)ccc1O ; COc1cc(-c2nc3c(Br)cccc3c(=O)n2C)ccc1O]

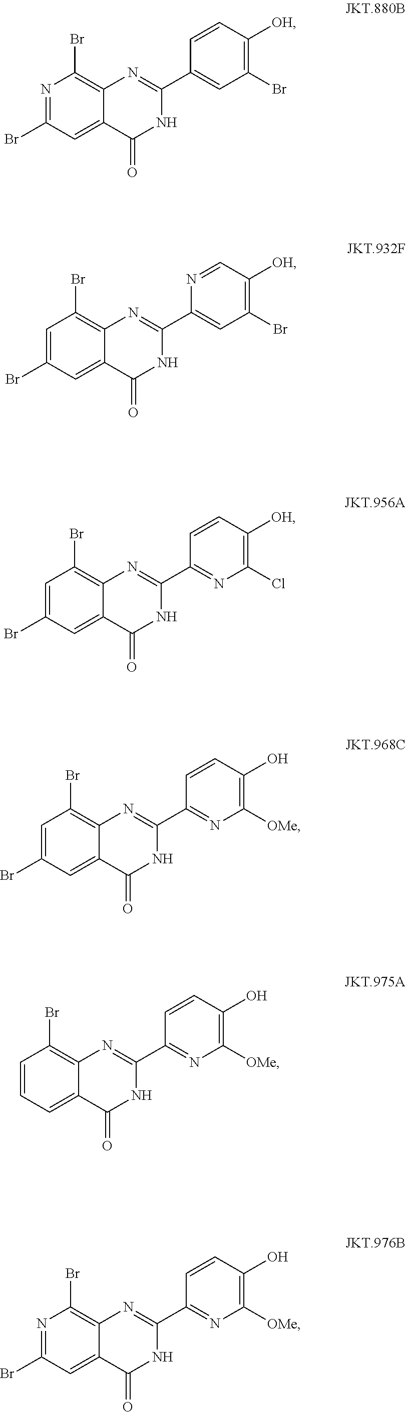 [SMILES: O=c1[nH]c(-c2ccc(O)c(Br)c2)nc2c(Br)nc(Br)cc12 ; O=c1[nH]c(-c2cc(Br)c(O)cn2)nc2c(Br)cc(Br)cc12 ; O=c1[nH]c(-c2ccc(O)c(Cl)n2)nc2c(Br)cc(Br)cc12 ; COc1nc(-c2nc3c(Br)cc(Br)cc3c(=O)[nH]2)ccc1O ; COc1nc(-c2nc3c(Br)cccc3c(=O)[nH]2)ccc1O ; COc1nc(-c2nc3c(Br)nc(Br)cc3c(=O)[nH]2)ccc1O]

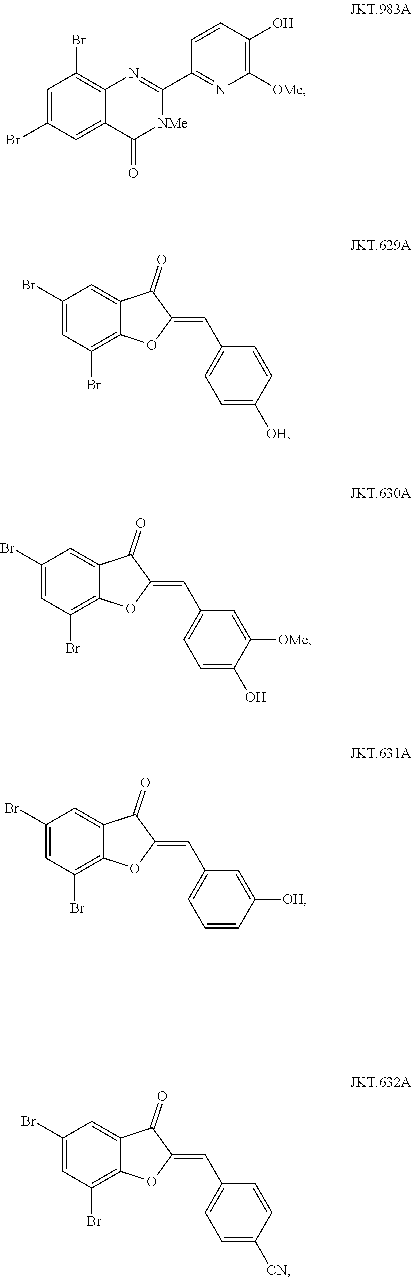 [SMILES: COc1nc(-c2nc3c(Br)cc(Br)cc3c(=O)n2C)ccc1O ; O=C1/C(=C/c2ccc(O)cc2)Oc2c(Br)cc(Br)cc21 ; COc1cc(/C=C2\Oc3c(Br)cc(Br)cc3C2=O)ccc1O ; O=C1/C(=C/c2cccc(O)c2)Oc2c(Br)cc(Br)cc21 ; N#Cc1ccc(/C=C2\Oc3c(Br)cc(Br)cc3C2=O)cc1]

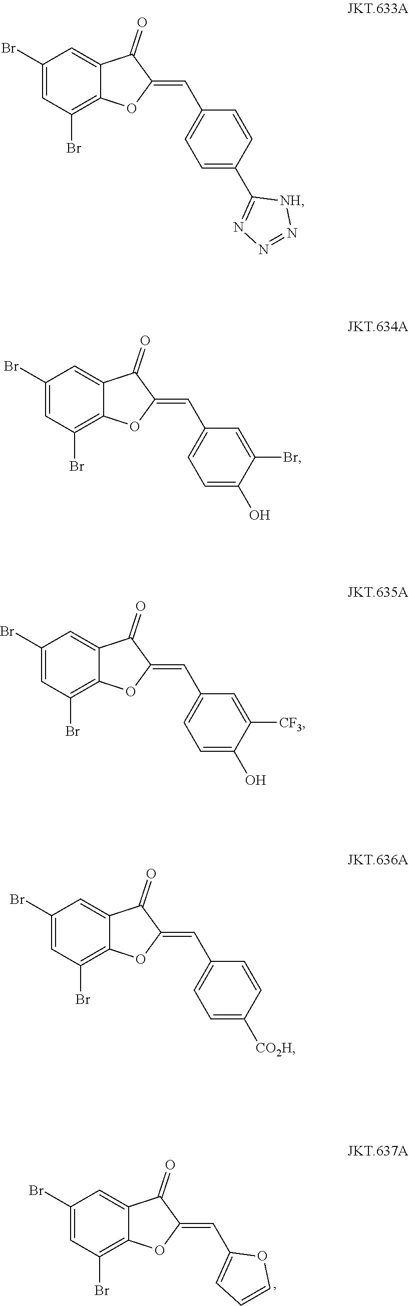 [SMILES: O=C1/C(=C/c2ccc(-c3nnn[nH]3)cc2)Oc2c(Br)cc(Br)cc21 ; O=C1/C(=C/c2ccc(O)c(Br)c2)Oc2c(Br)cc(Br)cc21 ; O=C1/C(=C/c2ccc(O)c(C(F)(F)F)c2)Oc2c(Br)cc(Br)cc21 ; O=C(O)c1ccc(/C=C2\Oc3c(Br)cc(Br)cc3C2=O)cc1 ; O=C1/C(=C/c2ccco2)Oc2c(Br)cc(Br)cc21]

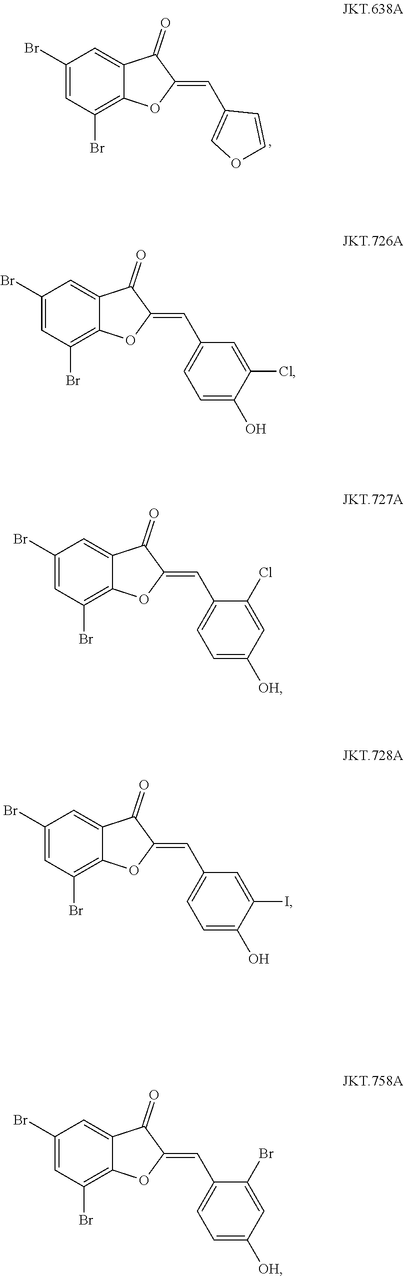 [SMILES: O=C1/C(=C/c2ccoc2)Oc2c(Br)cc(Br)cc21 ; O=C1/C(=C/c2ccc(O)c(Cl)c2)Oc2c(Br)cc(Br)cc21 ; O=C1/C(=C/c2ccc(O)cc2Cl)Oc2c(Br)cc(Br)cc21 ; O=C1/C(=C/c2ccc(O)c(I)c2)Oc2c(Br)cc(Br)cc21 ; O=C1/C(=C/c2ccc(O)cc2Br)Oc2c(Br)cc(Br)cc21]

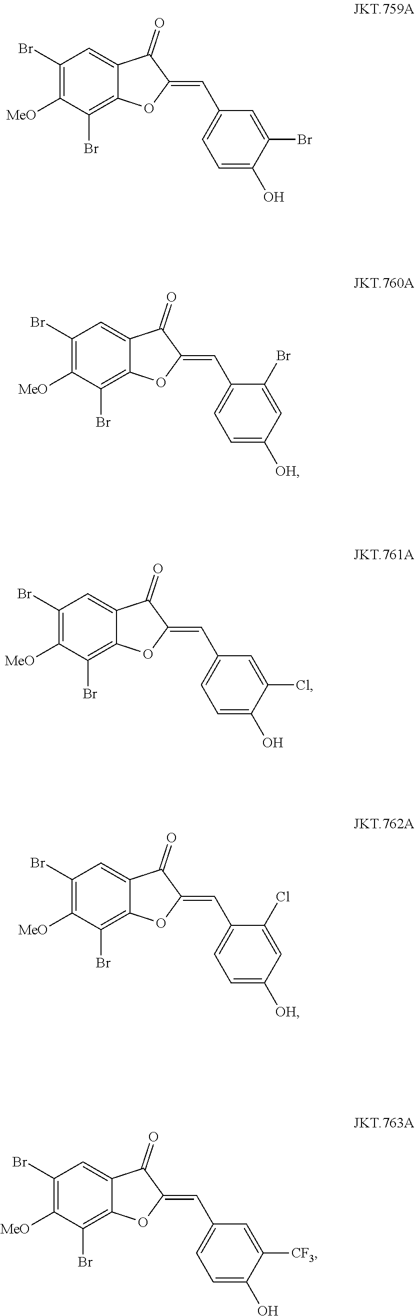 [SMILES: COc1c(Br)cc2c(c1Br)O/C(=C\c1ccc(O)c(Br)c1)C2=O ; COc1c(Br)cc2c(c1Br)O/C(=C\c1ccc(O)cc1Br)C2=O ; COc1c(Br)cc2c(c1Br)O/C(=C\c1ccc(O)c(Cl)c1)C2=O ; COc1c(Br)cc2c(c1Br)O/C(=C\c1ccc(O)cc1Cl)C2=O ; COc1c(Br)cc2c(c1Br)O/C(=C\c1ccc(O)c(C(F)(F)F)c1)C2=O]

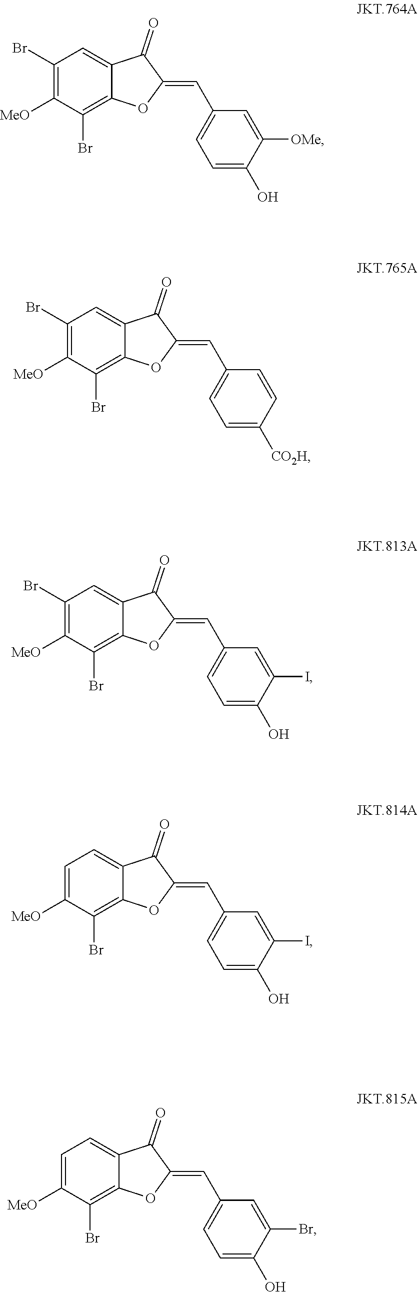 [SMILES: COc1cc(/C=C2\Oc3c(cc(Br)c(OC)c3Br)C2=O)ccc1O ; COc1c(Br)cc2c(c1Br)O/C(=C\c1ccc(C(=O)O)cc1)C2=O ; COc1c(Br)cc2c(c1Br)O/C(=C\c1ccc(O)c(I)c1)C2=O ; COc1ccc2c(c1Br)O/C(=C\c1ccc(O)c(I)c1)C2=O ; COc1ccc2c(c1Br)O/C(=C\c1ccc(O)c(Br)c1)C2=O]

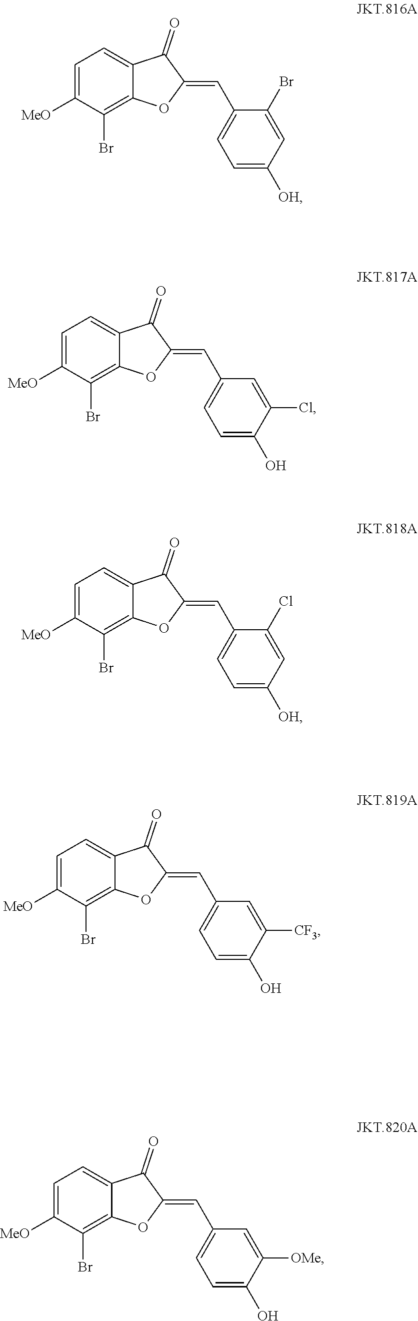 [SMILES: COc1ccc2c(c1Br)O/C(=C\c1ccc(O)cc1Br)C2=O ; COc1ccc2c(c1Br)O/C(=C\c1ccc(O)c(Cl)c1)C2=O ; COc1ccc2c(c1Br)O/C(=C\c1ccc(O)cc1Cl)C2=O ; COc1ccc2c(c1Br)O/C(=C\c1ccc(O)c(C(F)(F)F)c1)C2=O ; COc1cc(/C=C2\Oc3c(ccc(OC)c3Br)C2=O)ccc1O]

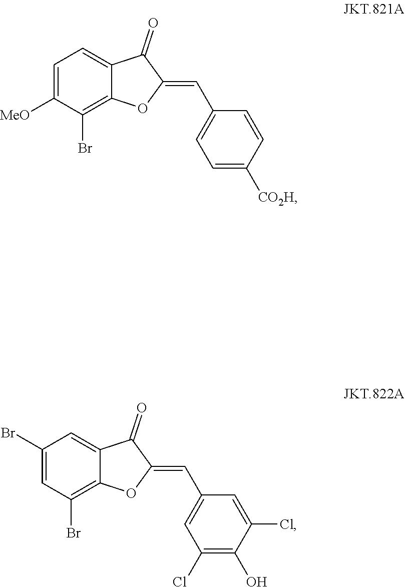 [SMILES: COc1ccc2c(c1Br)O/C(=C\c1ccc(C(=O)O)cc1)C2=O ; O=C1/C(=C/c2cc(Cl)c(O)c(Cl)c2)Oc2c(Br)cc(Br)cc21]

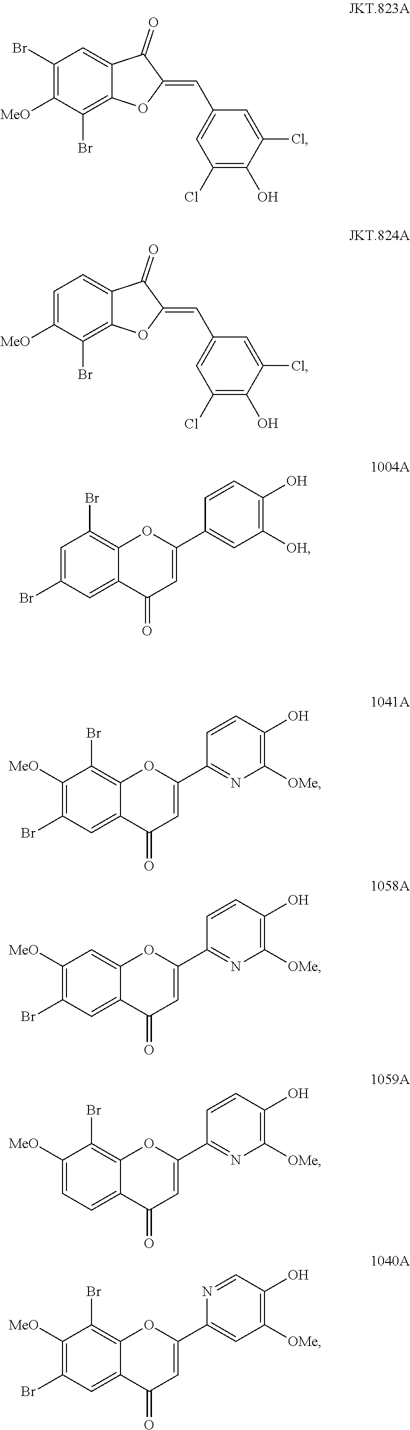

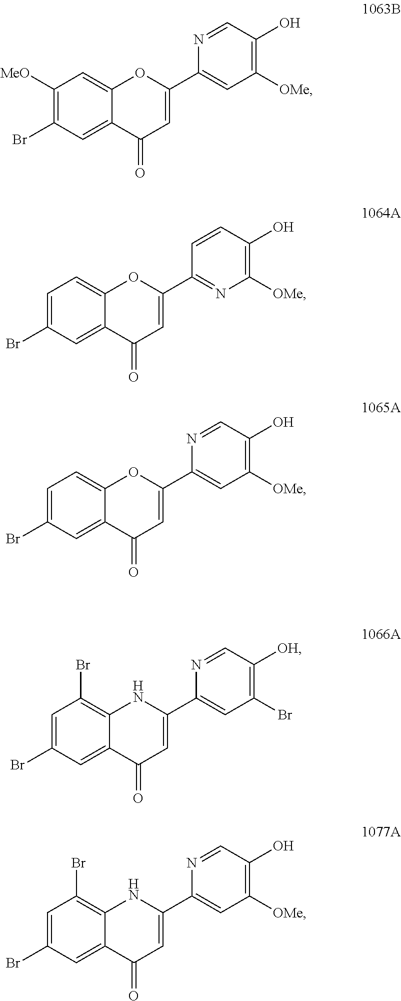 [SMILES: COc1cc(-c2cc(=O)c3cc(Br)c(OC)cc3o2)ncc1O ; COc1nc(-c2cc(=O)c3cc(Br)ccc3o2)ccc1O ; COc1cc(-c2cc(=O)c3cc(Br)ccc3o2)ncc1O ; O=c1cc(-c2cc(Br)c(O)cn2)[nH]c2c(Br)cc(Br)cc12 ; COc1cc(-c2cc(=O)c3cc(Br)cc(Br)c3[nH]2)ncc1O]

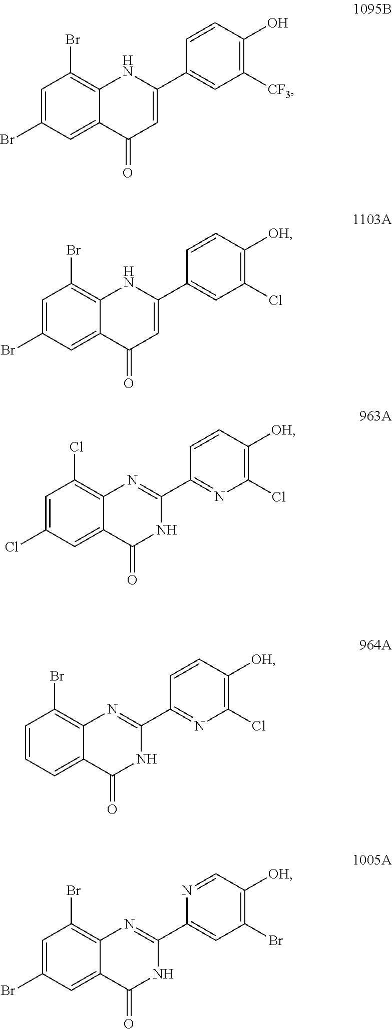 [SMILES: O=c1cc(-c2ccc(O)c(C(F)(F)F)c2)[nH]c2c(Br)cc(Br)cc12 ; O=c1cc(-c2ccc(O)c(Cl)c2)[nH]c2c(Br)cc(Br)cc12 ; O=c1[nH]c(-c2ccc(O)c(Cl)n2)nc2c(Cl)cc(Cl)cc12 ; O=c1[nH]c(-c2ccc(O)c(Cl)n2)nc2c(Br)cccc12 ; O=c1[nH]c(-c2cc(Br)c(O)cn2)nc2c(Br)cc(Br)cc12]

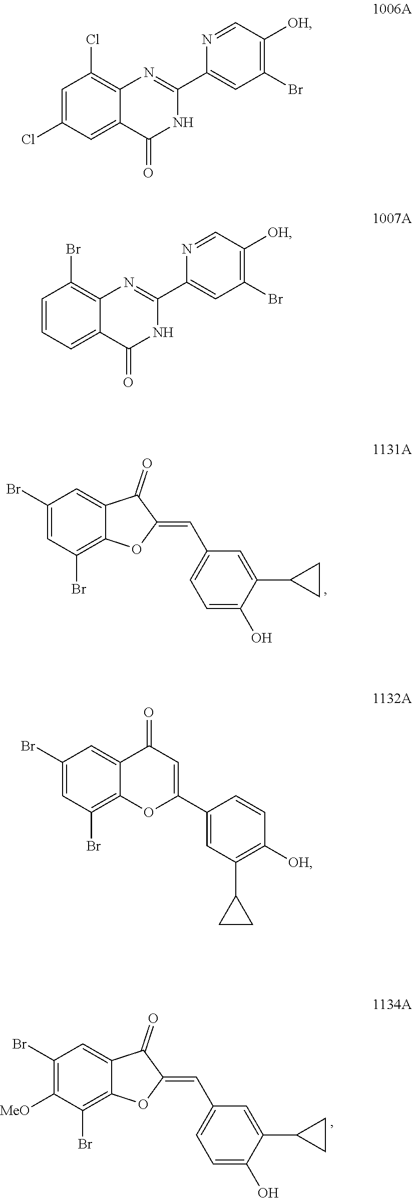 [SMILES: O=c1[nH]c(-c2cc(Br)c(O)cn2)nc2c(Cl)cc(Cl)cc12 ; O=c1[nH]c(-c2cc(Br)c(O)cn2)nc2c(Br)cccc12 ; O=C1/C(=C/c2ccc(O)c(C3CC3)c2)Oc2c(Br)cc(Br)cc21 ; O=c1cc(-c2ccc(O)c(C3CC3)c2)oc2c(Br)cc(Br)cc12 ; COc1c(Br)cc2c(c1Br)O/C(=C\c1ccc(O)c(C3CC3)c1)C2=O]

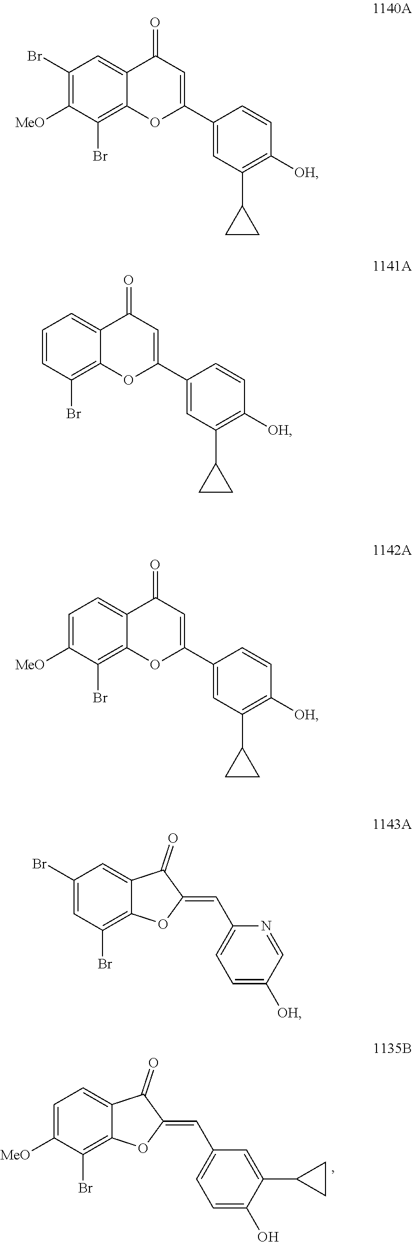 [SMILES: COc1c(Br)cc2c(=O)cc(-c3ccc(O)c(C4CC4)c3)oc2c1Br ; O=c1cc(-c2ccc(O)c(C3CC3)c2)oc2c(Br)cccc12 ; COc1ccc2c(=O)cc(-c3ccc(O)c(C4CC4)c3)oc2c1Br ; O=C1/C(=C/c2ccc(O)cn2)Oc2c(Br)cc(Br)cc21 ; COc1ccc2c(c1Br)O/C(=C\c1ccc(O)c(C3CC3)c1)C2=O]

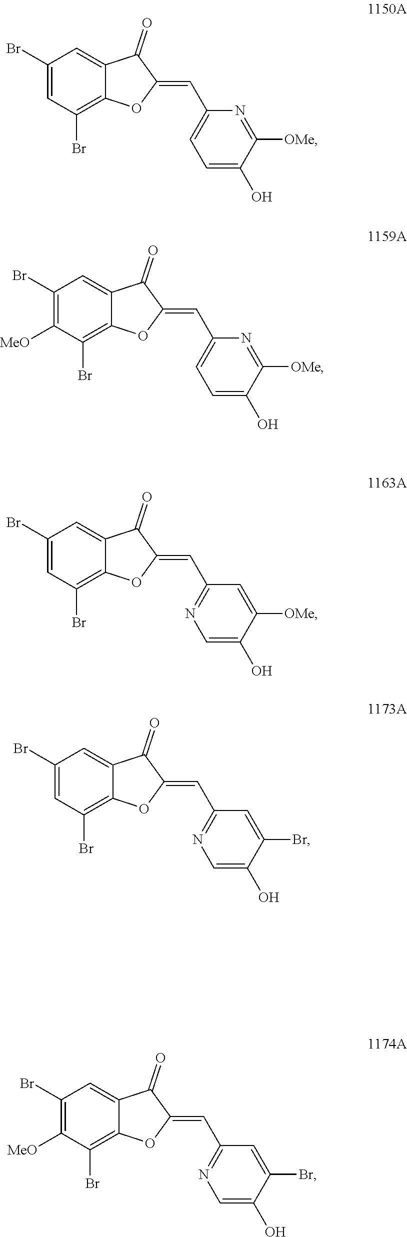 [SMILES: COc1nc(/C=C2\Oc3c(Br)cc(Br)cc3C2=O)ccc1O ; COc1nc(/C=C2\Oc3c(cc(Br)c(OC)c3Br)C2=O)ccc1O ; COc1cc(/C=C2\Oc3c(Br)cc(Br)cc3C2=O)ncc1O ; O=C1/C(=C/c2cc(Br)c(O)cn2)Oc2c(Br)cc(Br)cc21 ; COc1c(Br)cc2c(c1Br)O/C(=C\c1cc(Br)c(O)cn1)C2=O]

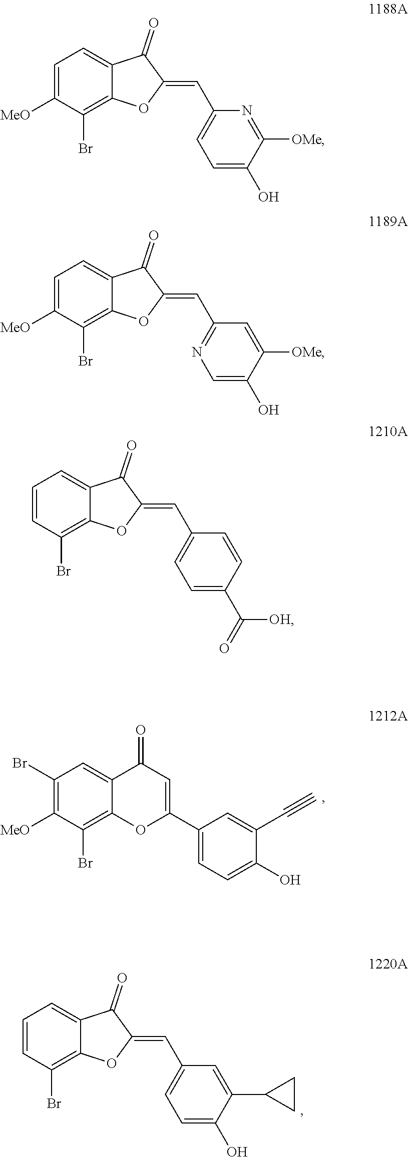 [SMILES: COc1ccc2c(c1Br)O/C(=C\c1ccc(O)c(OC)n1)C2=O ; COc1cc(/C=C2\Oc3c(ccc(OC)c3Br)C2=O)ncc1O ; O=C(O)c1ccc(/C=C2\Oc3c(Br)cccc3C2=O)cc1 ; C#Cc1cc(-c2cc(=O)c3cc(Br)c(OC)c(Br)c3o2)ccc1O ; O=C1/C(=C/c2ccc(O)c(C3CC3)c2)Oc2c(Br)cccc21]

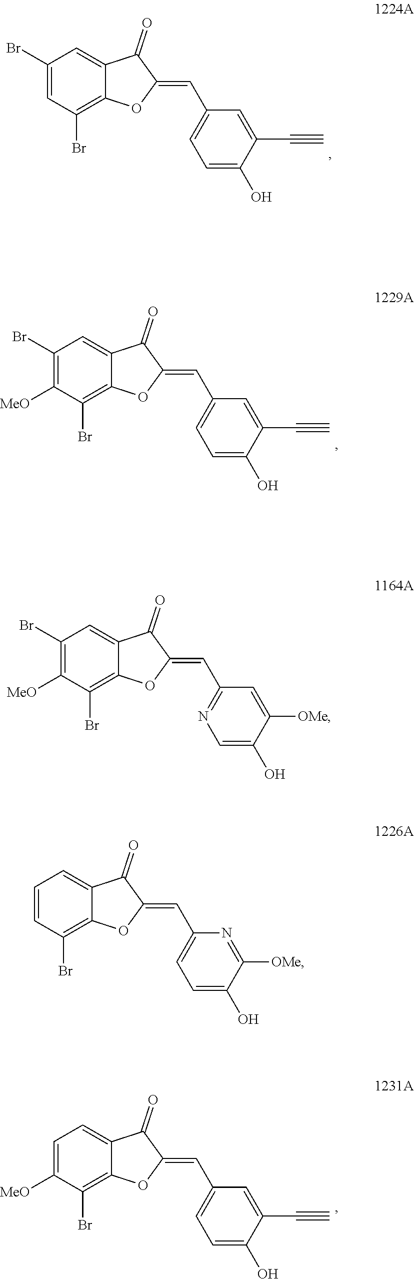 [SMILES: C#Cc1cc(/C=C2\Oc3c(Br)cc(Br)cc3C2=O)ccc1O ; C#Cc1cc(/C=C2\Oc3c(cc(Br)c(OC)c3Br)C2=O)ccc1O ; COc1cc(/C=C2\Oc3c(cc(Br)c(OC)c3Br)C2=O)ncc1O ; COc1nc(/C=C2\Oc3c(Br)cccc3C2=O)ccc1O ; C#Cc1cc(/C=C2\Oc3c(ccc(OC)c3Br)C2=O)ccc1O]

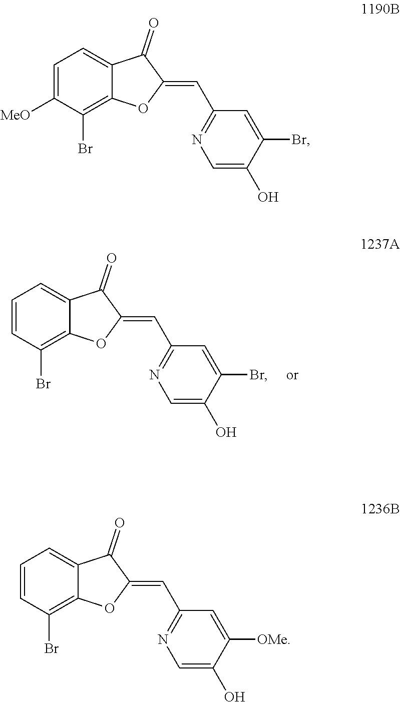 [SMILES: COc1ccc2c(c1Br)O/C(=C\c1cc(Br)c(O)cn1)C2=O ; O=C1/C(=C/c2cc(Br)c(O)cn2)Oc2c(Br)cccc21 ; COc1cc(/C=C2\Oc3c(Br)cccc3C2=O)ncc1O]

16. A compound, wherein the compound is:

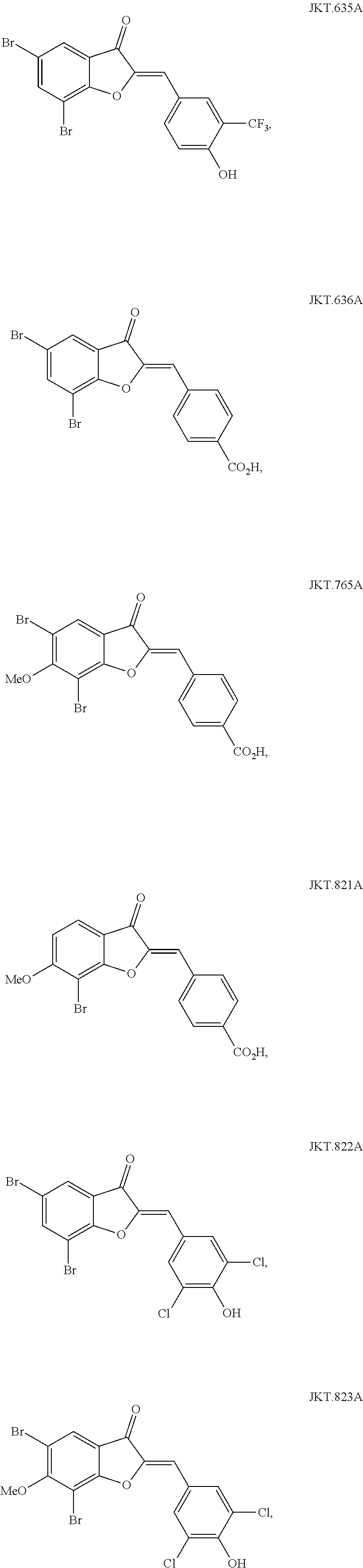 [SMILES: O=C1/C(=C/c2ccc(O)c(C(F)(F)F)c2)Oc2c(Br)cc(Br)cc21 ; O=C(O)c1ccc(/C=C2\Oc3c(Br)cc(Br)cc3C2=O)cc1 ; COc1c(Br)cc2c(c1Br)O/C(=C\c1ccc(C(=O)O)cc1)C2=O ; COc1ccc2c(c1Br)O/C(=C\c1ccc(C(=O)O)cc1)C2=O ; O=C1/C(=C/c2cc(Cl)c(O)c(Cl)c2)Oc2c(Br)cc(Br)cc21 ; COc1c(Br)cc2c(c1Br)O/C(=C\c1cc(Cl)c(O)c(Cl)c1)C2=O]

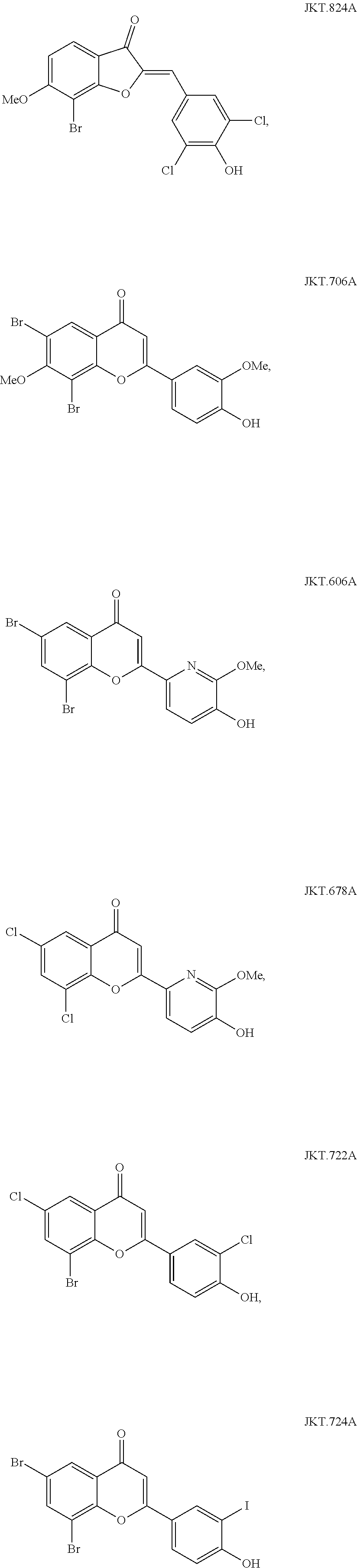 [SMILES: COc1ccc2c(c1Br)O/C(=C\c1cc(Cl)c(O)c(Cl)c1)C2=O ; COc1cc(-c2cc(=O)c3cc(Br)c(OC)c(Br)c3o2)ccc1O ; COc1nc(-c2cc(=O)c3cc(Br)cc(Br)c3o2)ccc1O ; COc1nc(-c2cc(=O)c3cc(Cl)cc(Cl)c3o2)ccc1O ; O=c1cc(-c2ccc(O)c(Cl)c2)oc2c(Br)cc(Cl)cc12 ; O=c1cc(-c2ccc(O)c(I)c2)oc2c(Br)cc(Br)cc12]

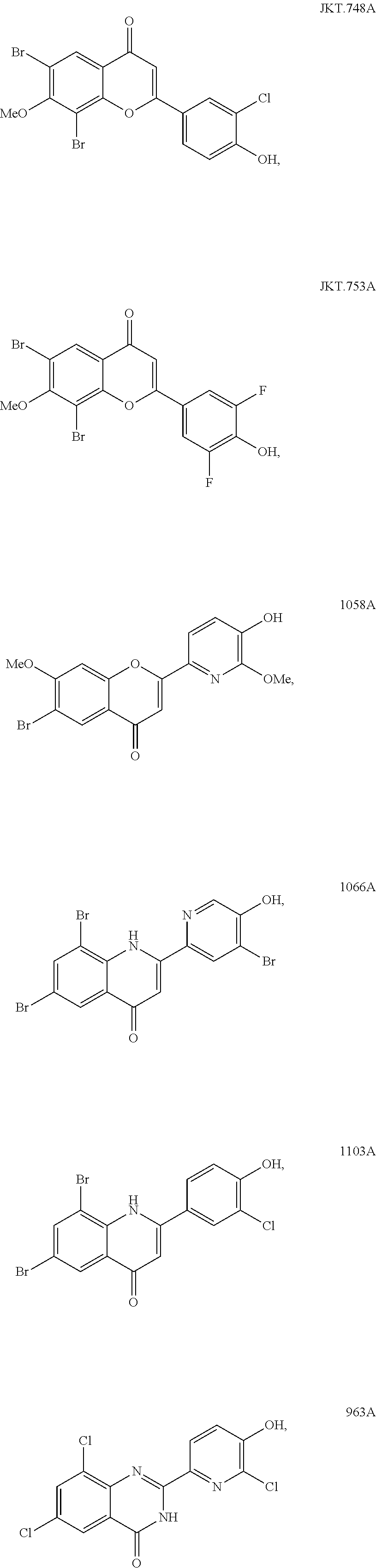 [SMILES: COc1c(Br)cc2c(=O)cc(-c3ccc(O)c(Cl)c3)oc2c1Br ; COc1c(Br)cc2c(=O)cc(-c3cc(F)c(O)c(F)c3)oc2c1Br ; COc1cc2oc(-c3ccc(O)c(OC)n3)cc(=O)c2cc1Br ; O=c1cc(-c2cc(Br)c(O)cn2)[nH]c2c(Br)cc(Br)cc12 ; O=c1cc(-c2ccc(O)c(Cl)c2)[nH]c2c(Br)cc(Br)cc12 ; O=c1[nH]c(-c2ccc(O)c(Cl)n2)nc2c(Cl)cc(Cl)cc12]

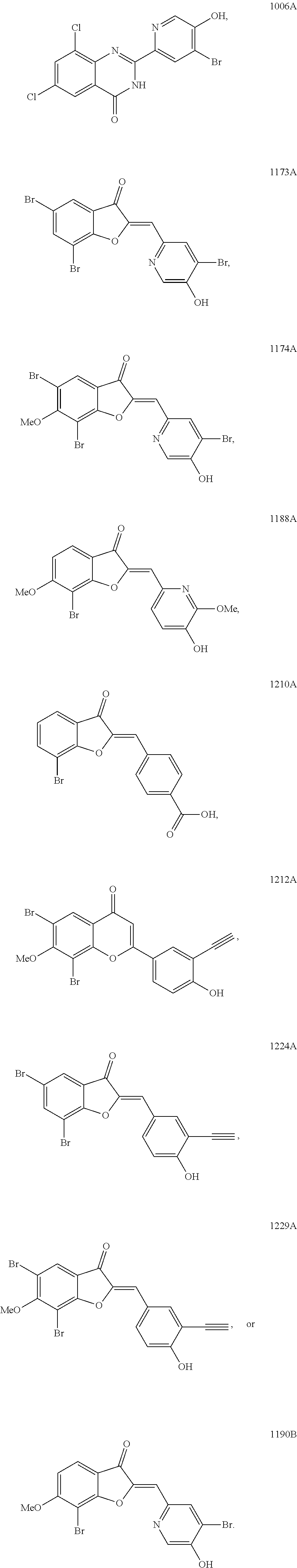

17. A pharmaceutical composition comprising a compound according to claim 1, or a pharmaceutically acceptable salt, hydrate, stereoisomer, or tautomer thereof, and a pharmaceutically acceptable excipient.

18. A method of reducing CK2 enzyme activity, comprising:
contacting a cell that expresses CK2 enzyme with an effective amount of one or more compounds according to claim 1, thereby reducing activity of the CK2 enzyme.

19-22. (canceled)

23. The method of claim 18, where contacting the cell with the one or more compounds comprises administering a therapeutically effective amount of the one or more compounds, or an amount of a pharmaceutical composition comprising the therapeutically effective amount of the one or more compounds, to a subject having a disease or condition characterized at least in part by dysregulated CK2 enzyme activity.

24-28. (canceled)

29. The method of claim 23, wherein the disease or condition is a neurodegenerative disease, cancer, cardiac hypertrophy, cystic fibrosis, bipolar disorder, depression, a viral infection, obesity, diabetes mellitus, atherosclerosis, epilepsy, or any combination thereof.

30. The method of claim 29, wherein the neurodegenerative disease is Alzheimer's disease, Huntington's disease, Parkinson's disease, multiple sclerosis, or amyotrophic lateral sclerosis.

31-36. (canceled)


SEQUENCE LISTING (4 sequences)

SEQ ID NO: 1 - DNA, 20 bp, synthetic construct
  source 1..20: mol_type = other DNA
gtgaggatagccaaggttct

SEQ ID NO: 2 - DNA, 20 bp, synthetic construct
  source 1..20: mol_type = other DNA
acgccgaggtgaacagtctg

SEQ ID NO: 3 - DNA, 20 bp, synthetic construct
  source 1..20: mol_type = other DNA
atctcctgaaaggaggccat

SEQ ID NO: 4 - DNA, 20 bp, synthetic construct
  source 1..20: mol_type = other DNA
agggcaggcgtcaccccctt